SEQUENCE LISTING (77 sequences)

SEQ ID NO: 1 - protein, 12 aa, Artificial Sequence
  Synthetic sequence
CQFDLSTRRLRC

SEQ ID NO: 2 - protein, 12 aa, Artificial Sequence
  Synthetic sequence
CQYNLSSRALKC

SEQ ID NO: 3 - protein, 12 aa, Artificial Sequence
  Synthetic sequence
  misc_feature (3)..(3): Xaa can be any naturally occurring amino acid
CQXDLSTRRLRC

SEQ ID NO: 4 - protein, 12 aa, Artificial Sequence
  Synthetic sequence
  misc_feature (4)..(4): Xaa can be any naturally occurring amino acid
CQFXLSTRRLRC

SEQ ID NO: 5 - protein, 12 aa, Artificial Sequence
  Synthetic sequence
  misc_feature (8)..(8): Xaa can be any naturally occurring amino acid
CQFDLSTXRLRC

SEQ ID NO: 6 - protein, 12 aa, Artificial Sequence
  Synthetic sequence
  misc_feature (9)..(9): Xaa can be any naturally occurring amino acid
CQFDLSTRXLRC

SEQ ID NO: 7 - protein, 12 aa, Artificial Sequence
  Synthetic sequence
  misc_feature (10)..(10): Xaa can be any naturally occurring amino acid
CQFDLSTRRXRC

SEQ ID NO: 8 - protein, 12 aa, Artificial Sequence
  Synthetic sequence
  misc_feature (3)..(3): Xaa can be any naturally occurring amino acid
CQXNLSSRALKC

SEQ ID NO: 9 - protein, 12 aa, Artificial Sequence
  Synthetic sequence
  misc_feature (4)..(4): Xaa can be any naturally occurring amino acid
CQYXLSSRALKC

SEQ ID NO: 10 - protein, 12 aa, Artificial Sequence
  Synthetic sequence
  misc_feature (5)..(5): Xaa can be any naturally occurring amino acid
CQYNXSSRALKC

SEQ ID NO: 11 - protein, 12 aa, Artificial Sequence
  Synthetic sequence
  misc_feature (8)..(8): Xaa can be any naturally occurring amino acid
CQYNLSSXALKC

SEQ ID NO: 12 - protein, 12 aa, Artificial Sequence
  Synthetic sequence
  misc_feature (9)..(9): Xaa can be any naturally occurring amino acid
CQYNLSSRXLKC

SEQ ID NO: 13 - protein, 12 aa, Artificial Sequence
  Synthetic sequence
  misc_feature (10)..(10): Xaa can be any naturally occurring amino acid
CQYNLSSRAXKC

SEQ ID NO: 14 - protein, 16 aa, Artificial Sequence
  Synthetic sequence
  misc_feature (3)..(3): Xaa can be any naturally occurring amino acid
CQXDLSTRRLRCGGGK

SEQ ID NO: 15 - protein, 16 aa, Artificial Sequence
  Synthetic sequence
  misc_feature (4)..(4): Xaa can be any naturally occurring amino acid
CQFXLSTRRLRCGGGK

SEQ ID NO: 16 - protein, 16 aa, Artificial Sequence
  Synthetic sequence
  misc_feature (8)..(8): Xaa can be any naturally occurring amino acid
CQFDLSTXRLRCGGGK

SEQ ID NO: 17 - protein, 16 aa, Artificial Sequence
  Synthetic sequence
  misc_feature (9)..(9): Xaa can be any naturally occurring amino acid
CQFDLSTRXLRCGGGK

SEQ ID NO: 18 - protein, 16 aa, Artificial Sequence
  Synthetic sequence
  misc_feature (10)..(10): Xaa can be any naturally occurring amino acid
CQFDLSTRRXRCGGGK

SEQ ID NO: 19 - protein, 16 aa, Artificial Sequence
  Synthetic sequence
  misc_feature (3)..(3): Xaa can be any naturally occurring amino acid
CQXNLSSRALKCGGGK

SEQ ID NO: 20 - protein, 16 aa, Artificial Sequence
  Synthetic sequence
  misc_feature (4)..(4): Xaa can be any naturally occurring amino acid
CQYXLSSRALKCGGGK

SEQ ID NO: 21 - protein, 16 aa, Artificial Sequence
  Synthetic sequence
  misc_feature (5)..(5): Xaa can be any naturally occurring amino acid
CQYNXSSRALKCGGGK

SEQ ID NO: 22 - protein, 16 aa, Artificial Sequence
  Synthetic sequence
  misc_feature (8)..(8): Xaa can be any naturally occurring amino acid
CQYNLSSXALKCGGGK

SEQ ID NO: 23 - protein, 16 aa, Artificial Sequence
  Synthetic sequence
  misc_feature (9)..(9): Xaa can be any naturally occurring amino acid
CQYNLSSRXLKCGGGK

SEQ ID NO: 24 - protein, 16 aa, Artificial Sequence
  Synthetic sequence
  misc_feature (10)..(10): Xaa can be any naturally occurring amino acid
CQYNLSSRAXKCGGGK

SEQ ID NO: 25 - protein, 214 aa, Artificial Sequence
  Synthetic sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 449 aa, Artificial Sequence
  Synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Artificial Sequence
  Synthetic sequence
DILLTQSPVILSVSPGERVSFSCRASQSIGTNIHWYQQRTNGSPRLLIKYASESISGIPSRFSGSGSGTDFTLSINSVESEDEADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 449 aa, Artificial Sequence; identical to SEQ ID NO: 26
  Synthetic sequence

SEQ ID NO: 29 - protein, 214 aa, Artificial Sequence
  Synthetic sequence
DIQMTQSPSILSASVGDRVTITCRASQDVNTAVAWYQQRTNKAPRLLIYSASFLYSGVPSRFSGSRSGTDFTLTISSLQPEDEADYYCQQHYTTPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 449 aa, Artificial Sequence
  Synthetic sequence
EVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, Artificial Sequence
  Synthetic sequence
DIQMTQSPSILSASVGDRVTITCQASQDISNYLNWYQQRTNKAPRLLIYDASNLETGVPSRFSGSGSGTDFTFTISSLQPEDEADYFCQHFDHLPLAFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 445 aa, Artificial Sequence
  Synthetic sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 214 aa, Artificial Sequence
  Synthetic sequence
DILMTQSPSIMSVSLGDTVSITCHSSQDINSNIGWLQQRTNGSPRGLIYHGTNLDDEVPSRFSGSGSGADYSLTISSLESEDEADYYCVQYAQFPWTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 445 aa, Artificial Sequence
  Synthetic sequence
DVQLQESGPSLVKPSQSLSLTCTVTGYSITSDFAWNWIRQSPGNKLEWMGYISYSGNTRYNPSLKSRISITRDTSKNQFFLQLNSVTIEDTAIYYCVTAGRGFPYWGQGTLVTVSASTKGPSVFPLAPSSKSTSGGTAALGCLVKDYFPEPVTVSWNSGALTSGVHTFPAVLQSSGLYSLSSVYSVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 213 aa, Artificial Sequence
  Synthetic sequence
QIVLTQSPAIMSASPGEKVTITCSASSSISYMHWFQQRTNGSPRLWIYTTSNLASGVPARFSGSGSGTSYSLTISRMEAEDEADYYCHQRSTYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, Artificial Sequence
  Synthetic sequence
QVQLQQSGAELAKPGASVKMSCKASGYTFTSYRMHWVKQSPGQGLEWIGYINPSTGYTEYNQKFKDKATLTADKSSSTAYMQLSSLTFEDSAIYYCARGGGVFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 218 aa, Artificial Sequence
  Synthetic sequence
DIQMTQSPSILSASVGDRVTITCRASESVDNYGISFMNWFQQRTNKAPRLLIYAASNQGSGVPSRFSGSGSGTDFTLTISSLQPDDEADYYCQQSKEVPWTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 446 aa, Artificial Sequence
  Synthetic sequence
EVQLVQSGAEVKKPGSSVKVSCKASGYTFTDYNMHWVRQSPGQGLEWIGYIYPYNGGTGYNQKFKSKATITADESTNTAYMELSSLRSEDTAIYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 218 aa, Artificial Sequence
  Synthetic sequence
GVHSDIQMTQSPSILSASVGDRVTITCSASQDIGNFLNWYQQRTNKTPRVLIYYTSSLYSGVPSRFSGSGSGTDYTLTISSLQPEDEADYYCQQYSKLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 454 aa, Artificial Sequence
  Synthetic sequence
GVHSQVQLVQSGAEVVKPGASVKMSCKASGYTFTGYNIHWVKQSPGQGLEWIGAIYPGNGDTSYKQKFRGRATLTADTSTSTVYMELSSLRSEDSAIYYCARGETARATFAYWGQGTLVTVSSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 215 aa, Artificial Sequence
  Synthetic sequence
DIQLTQSPSILSASVGDRVTMTCSASSSVSSSYLYWYQQKTNKAPRLWIYSTSNLASGVPARFSGSGSGTDFTLTISSLQPEDEADYFCHQWNRYPYTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 449 aa, Artificial Sequence
  Synthetic sequence
QVQLQQSGAEVKKPGASVKVSCEASGYTFPSYVLHWVKQSPGQGLEWIGYINPYNDGTQYNEKFKGKATLTRDTSINTAYMELSRLRSDDTAIYYCARGFGGSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 214 aa, Artificial Sequence
  Synthetic sequence
DILLTQSPVILSVSPGERVSFSCRASQSIGTNIHWYQQRTNGSPRLLIKYASESISGIPSRFSGSGSGTDFTLSINSVESEDEADYYCQQNNNWPTTFGAGTRLELKRTVAAPSVFIFPPSDEQLKSGTASVVCLLNNFYPREAKVQWKVDNALQSGNSQESVTDQDSKDSTYSLSSTLTLSKADYEKHKVYACEVTHQGLSSPVTKSFNRGEC

SEQ ID NO: 44 - protein, 449 aa, Artificial Sequence
  Synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGKGTLVTVSAASTKGPSVFPLAPSSKSTSGGTAALGCLVKDYFPSPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 214 aa, Artificial Sequence
  Synthetic sequence
DILLTQSPVILSVSPGERVSFSCRASQSIGTNIHWYQQRTNGSPRLLIKYASESISGIPSRFSGSGSGTDFTLSINSVESEDEADYYCQQNNNWPTTFGAGTQLELKRTVAAPSVFIFPPSDEQLKSGTASVVCLLNNFYPREAKVQWKVDNALQSGNSQESVTPQDSKDSTYSLSSTLTLSKADYEKHKVYACEVTHQGLSSPVTKSFNRGEC

SEQ ID NO: 46 - protein, 449 aa, Artificial Sequence
  Synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGSGTLVTVSAASTKGPSVFPLAPSSKSTSGGTAALGCLVKDYFPDPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 221 aa, Artificial Sequence
  Synthetic sequence
DIQMTQSPSILSASVGDRVTITCKSSQSLLYSSNQKIYLAWYQQKTNGAPKLLIYIWASTRESGVPSRFSGSGSGTDFTFTISSLQPEDEADYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 448 aa, Artificial Sequence
  Synthetic sequence
QVQLVQSGAEVKKPGASVKVSCKASGYTFSAYWIEWVRQSPGKGLEWVGEILPGSGYTRYNEKFKGRVTVTRDTSTNTAYMELSSLRANDTAIYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 211 aa, Artificial Sequence
  Synthetic sequence
EIVLTQSPAIMSASPGERVTMTCSASSGVNYMHWYQQKTNGSPRRWIYDTSKLASGVPARFSGSGSGTDYSLTISSMEPEDEADYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 450 aa, Artificial Sequence
  Synthetic sequence
QVQLVQPGAEVVKPGASVKLSCKTSGYTFTSNWMHWVKQSPGQGLEWIGEIDPSDSYTNYNQNFQGKAKLTVDKSTSTAYMEVSSLRSDDTAIYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 219 aa, Artificial Sequence
  Synthetic sequence
DIVMTQSPAILSLSPGERATLSCRSSKSLQNVNGNTYLYWFQQKTNGSPRLLIYRMSNLNSGVPDRFSGSGSGTEFTLTISSLEPEDEADYYCIQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 451 aa, Artificial Sequence
  Synthetic sequence
EVQLVESGGGLVKPGGSLKLSCAASGYTFTSYVMHWVRQSPGKGLEWIGYINPYNDGTKYNEKFQGRVTISSDKSISTAYMELSSLRSEDTAIYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - protein, 213 aa, Artificial Sequence
  Synthetic sequence
QIVLSQSPVILSASPGEKVTMTCRASSSVSYIHWFQQRTNGSPRPWIYATSNLASGVPVRFSGSGSGTSYSLTISRVEAEDEADYYCQQWTSNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 451 aa, Artificial Sequence
  Synthetic sequence
QVQLQQPGAELVKPGASVKMSCKASGYTFTSYNMHWVKQSPGRGLEWIGAIYPGNGDTSYNQKFKGKATLTADKSSSTAYMQLSSLTSEDSAIYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, Artificial Sequence
  Synthetic sequence
DIQMTQSPVILSASVGETVTITCRASENIHNYLAWYQQRTNGSPRLLVYNPKNLADGVPSRFSGSGSGTQYSLNINSLQPEDEGDYYCQHFWNTPPTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 451 aa, Artificial Sequence
  Synthetic sequence
EVQLQQSGPELVKPGASVQISCKTSGYTFTEYTMHWVKQSHGKSLEWLGIIHPDNGITRYNQKFKAKATLTEDKSSRTAYMELRSLTSEDSAIYYCARRYYGNFDYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 213 aa, Artificial Sequence
  Synthetic sequence
EIVLTQSPVILSLSPGERATLSCRASSSVPYIHWYQQRTNGSPRLLIYATSALASGIPDRFSGSGSGTDFTLTISRLEPEDEADYYCQQWLSNPPTFGAATKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 450 aa, Artificial Sequence
  Synthetic sequence
EVQLVQSGAEVKKPGESLKISCKGSGRTFTSYNMHWVRQSPGKGLEWMGAIYPLTGDTSYNQKSKLQVTISADKSISTAYLQWSSLKASDTAIYYCARSTYVGGDWQFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 214 aa, Artificial Sequence
  Synthetic sequence
DIQMTQSPVILSASVGDRVTITCRASQDVNTAVAWYQQRTNGSPKLLIYSASFLYSGVPSRFSGSRSGTDFTLTISSLQPEDEADYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 449 aa, Artificial Sequence
  Synthetic sequence
EVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 214 aa, Artificial Sequence
  Synthetic sequence
DILMTQSPVILSASVGDRVTINCKASQDINSYLSWYQQRTNGSPKTLIYRANRLVDGVPSRFSGSGSGQDYTLTISSLQPEDEADYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 415 aa, Artificial Sequence
  Synthetic sequence
EDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYPGDGSTKYNEKFKAKATLTADTSTSTAYMELRSLRSDDTAVYYCASGYEDAMDYWGQGTTVTVSSA

SEQ ID NO: 63 - protein, 214 aa, Artificial Sequence
  Synthetic sequence
DILMTQSPVILSASVGDRVTITCQASQDISNYLNWYQQRTNGSPKLLIYDASNLETGVPSRFSGSGSGTDFTFTISSLQPEDIADYYCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 445 aa, Artificial Sequence
  Synthetic sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 211 aa, Artificial Sequence
  Synthetic sequence
EIVLTQSPVILSASPGERVTITCSASSGVNYMHWYQQRTNGSPKRWIYDTSKLASGVPARFSGSGSGTDYSLTISSMEPEDEADYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 450 aa, Artificial Sequence; identical to SEQ ID NO: 50
  Synthetic sequence

SEQ ID NO: 67 - protein, 214 aa, Artificial Sequence
  Synthetic sequence
DIQMTQSPVILSASVGDRVTITCKASQDINNYLAWYQQRTNGSPKLLIHYTSTLHPGIPSRFSGSGSGRDYSFSISSLEPEDEADYYCLQYDNLLYTFGQGTKLEIKRTVAAPSVFIFPPSDEQLKSGTASVVSLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 448 aa, Artificial Sequence
  Synthetic sequence
QVQLVQSGAEVAKPGASVKLSCKASGYTFTSYWMQWVKQRPGQGLECIGTIYPGDGDTTYTQKFQGKATLTADKSSSTAYMQLSSLRSEDSAIYYCARYDAPGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSWRQQGNVFSCSVMHEALHNHYTQKSLSLSPG

SEQ ID NO: 69 - protein, 6 aa, Artificial Sequence
  Synthetic sequence
LCTPSR

SEQ ID NO: 70 - protein, 13 aa, Artificial Sequence
  Synthetic sequence
LCTPSRGSLFTGR

SEQ ID NO: 71 - protein, 15 aa, Artificial Sequence
  Synthetic sequence
CQFDLSTRRLCGGGK

SEQ ID NO: 72 - protein, 19 aa, Artificial Sequence
  Synthetic sequence
KGGGCQFDLSTRRLCGGGK

SEQ ID NO: 73 - protein, 28 aa, Artificial Sequence
  Synthetic sequence
KGGGCQFDLSTRRLCGGGKGGKSGGAGK

SEQ ID NO: 74 - protein, 28 aa, Artificial Sequence
  Synthetic sequence
CQFDLSTRRLCGGGKGGASGGAGKGGGK

SEQ ID NO: 75 - protein, 27 aa, Artificial Sequence
  Synthetic sequence
CQFDLSTRRLCGGGKGGASGGGGSAGK

SEQ ID NO: 76 - protein, 20 aa, Artificial Sequence
  Synthetic sequence
  misc_feature (14)..(14): Xaa can be any naturally occurring amino acid
KGGGCQFDLSTRRXRCGGGK

SEQ ID NO: 77 - protein, 16 aa, Artificial Sequence
  Synthetic sequence
  misc_feature (5)..(5): Xaa can be any naturally occurring amino acid
CQFDXSTRRLRCGGGK

(19) United States
(12) Patent Application Publication
(10) Pub. No.: US 2019/0111149 A1
(43) Pub. Date: Apr. 18, 2019

(54) ANTI-EGFR ANTIBODY DRUG CONJUGATE

(71) Applicant: Meditope Biosciences, Inc., Pasadena, CA (US)

(72) Inventors: Elisabeth Gardiner, Pasadena, CA (US); Calin D. Dumitru, Pasadena, CA (US); Michael H. Matho, Pasadena, CA (US)

(21) Appl. No.: 16/074,763

(22) PCT Filed: Feb. 2, 2017
(86) PCT No.: PCT/US17/16264

§ 371 Date: Aug. 1, 2018

Related U.S. Application Data
(60) Provisional application No. 62/290,382, filed on Feb. 2, 2016.

(51) Int. Cl. A61K 47/68; C07K 19/00; C07K 16/28; A61K 47/64

(52) U.S. Cl. CPC A61K 47/6849; C07K 19/00; C07K 16/2863; C07K 2317/24; A61K 47/64; A61K 47/6803; C07K 2317/92; A61K 47/6851

(57) ABSTRACT

The present disclosure is directed to meditope-antibody covalent binding modalities that can be activated only after the meditope associates with a binding site on the meditope-enabled antibody. To this end, provided herein is a meditope engineered to contain a photoreactive functional group at one or more specific location(s) on the meditope so as not interfere with the specific noncovalent molecular interaction with the binding site on the meditope-enabled antibody, but still permits, and enhances, covalent bond formation. This methodology, with its speed, accuracy, consistency and simplicity has evident advantages for the development of antibody-drug conjugates.

### CROSS REFERENCE TO RELATED APPLICATIONS

This application claims the benefit under 35 U.S.C. § 119(e) to U.S. Provisional Application No. 62/290,382, filed Feb. 2, 2016, the contents of which is incorporated by reference in its entirety.

### FIELD

The present disclosure is directed to meditope-antibody covalent binding modalities that can be activated only after the meditope associates with a binding site on the meditope-enabled antibody.

### BACKGROUND

Due to their specificity and favorable pharmacokinetics and pharmacodynamics, there have been substantial efforts to arm monoclonal antibodies (rnAbs), either with potent cytotoxins or biologics, to enhance their therapeutic efficacy or with radionuclides to image disease. These methods may be limited by available chemistries of the parental rnAb and/or require extensive protein engineering.

### SUMMARY

The present disclosure is directed to meditope-antibody covalent binding modalities that can be activated only after the meditope associates with a binding site on the meditope-enabled antibody. To this end, provided herein is a meditope engineered to contain a photoreactive functional group at one or more specific location(s) on the meditope so as not interfere with the specific noncovalent molecular interaction with the binding site on the meditope-enabled antibody, but still permits, and enhances, covalent bond formation. This methodology, with its speed, accuracy, consistency and simplicity has evident advantages for the development of antibody-drug conjugates.

In certain embodiments, provided is a non-covalent antibody-meditope complex, comprising a meditope comprising an amino acid having a photoreactive functional group capable of forming a covalent bond upon irradiation with UV light and an antibody comprising a meditope-enabled cavity.

In certain embodiments, provided is an antibody-meditope conjugate, comprising a meditope and an antibody comprising a meditope-enabled cavity, wherein a side chain of an amino acid of the meditope is bonded to the antibody, and wherein the covalent bond between the antibody and the meditope is not a disulfide bond. The antibodies of the antibody-meditope conjugate comprise a meditope-enabled cavity which contains amino acid residues that interact with a meditope, allowing the meditope to bind. In certain embodiments, the antibody has been modified in such a way that the meditope binds more efficiently (with a higher binding constant). In one embodiment, the antibody is cetuximab or a functional fragment of cetuximab comprising the meditope-enabled cavity.

In certain embodiments, provided is an antibody-meditope conjugate, comprising a cyclic meditope comprising the amino acid sequence of CQFDLSTRRXRC (SEQ ID NO: 7) or CQYNLSSRAXKC (SEQ ID NO: 13), or SEQ ID NO: 7 or 13 with one or two amino acid additions, deletions and/or substitutions and an antibody comprising a meditope-enabled cavity, wherein the side chain of amino acid X of the meditope is covalently bonded to the antibody, and wherein a disulfide bridge between C1 and C12 is closer to the entrance of the meditope-enabled cavity than the majority of amino acids 2-11 of the meditope.

In certain embodiments, provided is an antibody-meditope conjugate, comprising a meditope comprising the amino acid sequence of CQFDLSTRRXRC (SEQ ID NO: 7) or CQYNLSSRAXKC (SEQ ID NO: 13), with one or two amino acid additions, deletions and/or substitutions and an antibody comprising a meditope-enabled cavity, wherein the side chain of amino acid X of the meditope is covalently bonded to the antibody.

The side chain of amino acid X of the meditope may be covalently bonded to the antibody at any position, including the antibody backbone and/or an amino acid side chain of the antibody.

In certain embodiments, the side chain of X is covalently bonded to the antibody via a linker. In some embodiments, the linker is of the formula -L- or -L-L-, wherein each L is independently substituted or unsubstituted alkylene, substituted or unsubstituted cycloalkylene, substituted or unsubstituted arylene, substituted or unsubstituted heterocyclylene or substituted or unsubstituted heteroarylene.

In certain embodiments, the meditope further comprises at least one active agent, such as a therapeutic agent, a diagnostic agent, and/or a detectable agent, covalently bound thereto, optionally via a linker. The active agent can be bound to the meditope at the N-terminus, the C-terminus or off a side chain of an amino acid of the meditope. In certain embodiments, the meditope comprises more than one active agent covalently bound thereto. In such instances, the active agents can be covalently bound to both the N-terminus and the C-terminus, or the meditope can comprise branching such that the meditope comprises more than one N-terminus and/or C-terminus. The active agent can be covalently bound to the meditope directly or optionally via a linker.

Also provided is a peptide comprising the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Phe3, Asp4, Arg8, Arg9, and/or Leu10, and further wherein the modification comprises incorporation of a photoreactive functional group capable of forming a covalent bond upon irradiation with UV light.

Also provided is a peptide comprising the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Leu5, Arg8, Arg9, and/or Leu10, and further wherein the modification comprises incorporation of a photoreactive functional group capable of forming a covalent bond upon irradiation with UV light.

Also provided is a peptide comprising the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Phe3, Leu5, Arg8, Arg9, and/or Leu10, and further wherein the modification comprises incorporation of a photoreactive functional group capable of forming a covalent bond upon irradiation with UV light.

Also provided is a peptide comprising the amino acid sequence of CQYNLSSRALKC (SEQ ID NO: 2) or SEQ ID NO: 2 with one or two amino acid additions, deletions and/or substitutions, wherein the amino acid sequence comprises one or more modifications of at least one amino acid residue selected from the group consisting of Tyr3, Asn4, Leu5, Arg8, Ala9, and/or Leu10, and further wherein the modification comprises incorporation of a photoreactive functional group capable of forming a covalent bond upon irradiation with UV light.

Also provided is a peptide comprising the amino acid sequence of CQFDLSTRRX¹RC (SEQ ID NO: 7) or CQYNLSSRAX¹KC (SEQ ID NO: 13), or SEQ ID NO: 7 or 13 with one or two amino acid additions, deletions and/or substitutions, wherein amino acid X¹ comprises a photoreactive functional group capable of forming a covalent bond upon irradiation with UV light.

Also provided is a method of preparing an antibody-meditope conjugate, comprising contacting a cyclic meditope comprising the amino acid sequence of CQFDLSTRRX¹RC (SEQ ID NO: 7) or CQYNLSSRAX¹KC (SEQ ID NO: 13), or SEQ ID NO: 7 or 13 with one or two amino acid additions, deletions and/or substitutions with an antibody comprising a meditope-enabled cavity, wherein the side chain of amino acid X¹ of the meditope comprises a photoreactive functional group capable of forming a covalent bond upon irradiation with UV light under conditions to allow formation of a non-covalent interaction between the meditope and one or more amino acids in the meditope-enabled cavity, wherein a disulfide bridge between C1 and C12 is closer to the entrance of the meditope-enabled cavity than the majority of amino acids 2-11 of the meditope; and irradiating the meditope with UV light to generate a covalent bond between the side chain of amino acid X¹ of the meditope and the antibody.

### BRIEF DESCRIPTION OF THE DRAWINGS

FIG. 1, panels A-H, show the formation of the covalent meditope-antibody conjugates.

FIG. 2 shows that cells which express EGFR are effectively killed by the EGFR antibody drug conjugate (EGFR antibody coupled to a toxic auristatin (MMAD) via a covalently linking meditope peptide with a photo activatable methionine at position 10 of the CQFD peptide).

FIG. 3 shows that various meditope drug conjugates and meditope-antibody conjugates do not decrease body weight in mice at the doses tested.

FIG. 4 shows EGFR efficacy data and tumor volume reduction using cetuximab I83E with the meditope drug conjugate photo-Met 10 MMAD.

FIG. 5 is a modeling of a meditope in the meditope-binding cavity of cetuximab and meditope-enabled trastuzumab.

FIG. 6 shows UV-conjugation of a photo-optimized trastuzumab variant conjugated with photo enabled meditopes.

FIG. 7 shows cetuximab I83E conjugated with meditopes with different DAR.

FIG. 8 shows binding affinity of various meditopes.

### DETAILED DESCRIPTION

### 1. Definitions

Unless defined otherwise, all technical and scientific terms used herein have the same meaning as commonly understood by one of ordinary skill in the art to which this disclosure belongs. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

It must be noted that as used herein and in the appended claims, the singular forms “a”, “an”, and “the” include plural referents unless the context clearly dictates otherwise. Thus, for example, reference to “a peptide” includes a plurality of peptides.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The abbreviations used herein have their conventional meaning within the chemical and biological arts. The following abbreviations used herein have the following meanings.

| | |
|---|---|
| MMAD | Monomethyl auristatin D |
| PBD | Pyrrolobenzodiazepine |
| DM1 | Mertansine |
| MCC | Maleimidomethyl cyclohexane-1-carboxylate |
| PEG4 | 4-unit polyethylene glycol |
| PAB | Para-aminobenzyloxycarbonyl |
| MBI | Meditope Biosciences, Incorporated |
| DAR | Drug antibody ratio |
| Photo-Met 10 | Photoactivatable methionine at meditope C-QFD |
| or | peptide at position 10 (replaces leucine) |
| photoMet10 | |
| CDR | Complementarity determining regions |
| UV | Ultraviolet |
| EGFR | Epidermal growth factor |
| FR | Framework |
| Fab | Fragment antigen-binding |
| mAb | Monoclonal antibody |
| AFP | Dimethylvaline-valine-dolaisoleuinedolaproine- |
| | phenylalanine-p-phenylenediamine |
| MMAF | Dovaline-valine-dolaisoleuinedolaproine- |
| | phenylalanine |
| MMAE | Monomethyl auristatin E |
| BCG | Bacillus calmette-guerin |
| BOC | tert-Butyloxycarbonyl |
| Fmoc | Fluorenylmethyloxycarbonyl |
| i-Bu | Iso-Butyl |
| HPLC | High Performance Liquid Chromatography |
| min | Minute |
| TFA | Trifluoroacetic acid |
| DMSO | Dimethylsulfoxide |
| DMA | Dimethylacetate |
| BLI | Bio-Layer Interferometry |
| mpk | Milligrams per kilogram |
| SD | Standard deviation |
| scFv | Single-chain variable fragment |

As used herein, the term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies as used herein may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of 150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fe region ensures that each antibody generates an appropriate immune response for a given antigen. The Fe region also binds to various cell receptors, such as Fe receptors, and other immune molecules, such as complement proteins.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CHI by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is an antibody that typically is a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a monoclonal antibody (mAb) is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the disclosure and for use according to the disclosure, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this disclosure. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the disclosure include humanized and/or chimeric monoclonal antibodies.

A “therapeutic antibody” as provided herein refers to any antibody or functional fragment thereof that is used to treat cancer, autoimmune diseases, transplant rejection, cardiovascular disease or other diseases or conditions such as those described herein. Non-limiting examples of therapeutic antibodies include murine antibodies, murinized or humanized chimera antibodies or human antibodies including, but not limited to, Erbitux (cetuximab), ReoPro (abciximab), Simulect (basiliximab), Remicade (infliximab); Orthoclone OKT3 (muromonab-CD3); Rituxan (rituximab), Bexxar (tositumomab) Humira (adalimumab), Campath (alemtuzumab), Simulect (basiliximab), Avastin (bevacizumab), Cimzia (certolizumab pegol), Zenapax (daclizumab), Soliris (eculizumab), Raptiva (efalizumab), Mylotarg (gemtuzumab), Zevalin (ibritumomab tiuxetan), Tysabri (natalizumab), Xolair (omalizumab), Synagis (palivizumab), Vectibix (panitumumab), Lucentis (ranibizumab), and Herceptin (trastuzumab).

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84; Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). A “therapeutic agent” as referred to herein, is a composition useful in treating or preventing a disease such as cancer.

The phrase “specifically binds” to an antibody when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “meditope” as used herein, refers to a peptide that binds to a meditope-binding site of a meditope-enabled antibody. Exemplary meditopes include, but are not limited to, SEQ ID NO: 1-24 as described herein and those disclosed in, e.g., U.S. Pat. Nos. 8,658,774 and 8,962,804 and variants thereof, as well as multivalent and labeled meditopes.

The term “meditope-enabled antibody” refers to an antibody or functional fragment thereof that is able to bind to a meditope, via a meditope binding site. Examples of meditope-enabled antibodies include, but are not limited to, cetuximab, meditope-enabled trastuzumab (see, e.g., U.S. Pat. No. 8,962,804), meditope-enabled M5A (see, e.g., U.S. Pat. No. 8,962,804) and others described herein. Methods of preparing meditope-enabled antibodies are known in the art, such as by grafting and those described in U.S. Pat. No. 8,962,804.

A “meditope-enabled cavity” is a region of the meditope-enabled antibody containing the amino acid residues that interact with a bound meditope, which residues include framework region (FR) residues of the heavy and light chains. With reference to a Fab fragment or a Fab portion of an antibody, the meditope-binding site is located within the central cavity of the Fab fragment or portion. The “central cavity,” with respect to the three-dimensional structure of a Fab, refers to the internal cavity of the Fab, lined by portions of the heavy and light chain variable and constant regions. The central cavity thus is lined by residues of the VH, VL, CHI, and CL regions and does not include the antigen binding site.

In certain embodiments, the meditope-enabled cavity is lined by residues of the VH, VL, CHI, and CL regions, respectively, and does not include the antigen binding site. In certain embodiments, the meditope-enabled cavity is lined by amino acid residues capable of interacting with the compound provided herein including embodiments thereof. In embodiments, the amino acid residues lining the central cavity include a residue at a position corresponding to Kabat position 40, a residue at a position corresponding to Kabat position 41, and a residue at a position corresponding to Kabat position 10. In embodiments, the amino acid residues lining the meditope-enabled cavity include a residue at a position corresponding to Kabat position 83. In embodiments, the amino acid residues lining the meditope-enabled cavity include a residue at a position corresponding to Kabat position 85. In embodiments, the amino acid residues lining the meditope-enabled cavity include residues forming a peptide binding site as described in published U.S. application no. US 2012/0301400, which is hereby incorporated by reference in its entirety and for all purposes.

As used herein, the term “antibody-meditope conjugate” refers to a covalently bound complex between an antibody and a meditope.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl” and “peptidyl moiety” means a monovalent peptide.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O— methyl ribonucleotides, and peptide-nucleic acids (PNAs).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the disclosure or individual domains of the polypeptides of the disclosure), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present disclosure includes peptides that are substantially identical to any of SEQ ID NOs: 1-77.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Afath. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in, Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl and the like. An unsaturated alkyl group is one having one or more double bonds or triple bonds are referred to as “alkenyl” and alkynyl”, respectively. Examples include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An “alkoxy” is an alkyl attached to the remainder of the molecule via an oxygen linker (—O—). An alkyl moiety may be an alkenyl moiety.

The term “alkylene,” by itself or as part of another substituent, means, unless otherwise stated, a divalent radical derived from an alkyl. Typically, an alkyl (or alkylene) group will have from 1 to 24 carbon atoms, with those groups having 10 or fewer carbon atoms being preferred in the present disclosure. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂CH₂—N(CH₃)—CH₃, —CH₂—S—CH₂CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of“alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C1-C4)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl”, “cycloalkyl”, “heterocycloalkyl”, “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂N(R′)(R″—NRSO₂R′), —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-4 halogens), substituted or unsubstituted alkyl, alkoxy, or thioalkoxy groups, or arylalkyl groups. When a compound of the disclosure includes more than one R group, for example, each of the R groups is independently selected as are each R′, R″, R, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, NR″C(O)₂R′, NRC(NR′R″)═NR′″, S(O)R′, —S(O)₂R′, —S(O)₂N(R′)(R″), —NRSO₂R′), —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the disclosure includes more than one R group, for example, each of the R groups is independently selected as are each R′, R″, R′″, and R″″ groups when more than one of these groups is present.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{IA}-substituted or unsubstituted alkyl, a plurality of R^{IA} substituents may be attached to the alkyl moiety wherein each R^{IA} substituent is optionally different. Where an R-substituted moiety is substituted with a plurality R substituents, each of the R-substituents may be differentiated herein using a prime symbol (′) such as R′, R″, etc. For example, where a moiety is R^{IA}-substituted or unsubstituted alkyl, and the moiety is substituted with a plurality of R^{IA} substituents, the plurality of R^{IA} substituents may be differentiated as R^{IA′}, R^{IA″}, R^{IA′″}, etc. In some embodiments, the plurality of R substituents is 3. In some embodiments, the plurality of R substituents is 2.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where variables s and d are independently integers of from 0 to 3, and X¹ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent” as used herein means a group selected from the following moieties: (A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and alkyl, heteroalkyl, cycloalkyl, heterocycloalkyl, aryl, heteroaryl, substituted with at least one substituent selected from:

(i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH2, —ONH2, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C1-C20 alkyl, each substituted or unsubstituted heteroalkyl is a substituted or unsubstituted 2 to 20 membered heteroalkyl, each substituted or unsubstituted cycloalkyl is a substituted or unsubstituted C₃-C8 cycloalkyl, each substituted or unsubstituted heterocycloalkyl is a substituted or unsubstituted 3 to 8 membered heterocycloalkyl, each substituted or unsubstituted aryl is a substituted or unsubstituted C6-C10 aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C1-C8 alkyl, each substituted or unsubstituted heteroalkyl is a substituted or unsubstituted 2 to 8 membered heteroalkyl, each substituted or unsubstituted cycloalkyl is a substituted or unsubstituted C3-C7 cycloalkyl, each substituted or unsubstituted heterocycloalkyl is a substituted or unsubstituted 3 to 7 membered heterocycloalkyl, each substituted or unsubstituted aryl is a substituted or unsubstituted C6-C10 aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C1-C20 alkyl, each substituted or unsubstituted heteroalkyl is a substituted or unsubstituted 2 to 20 membered heteroalkyl, each substituted or unsubstituted cycloalkyl is a substituted or unsubstituted C3-C8 cycloalkyl, each substituted or unsubstituted heterocycloalkyl is a substituted or unsubstituted 3 to 8 membered heterocycloalkyl, each substituted or unsubstituted aryl is a substituted or unsubstituted C6-C10 aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C1-C20 alkylene, each substituted or unsubstituted heteroalkylene is a substituted or unsubstituted 2 to 20 membered heteroalkylene, each substituted or unsubstituted cycloalkylene is a substituted or unsubstituted C3-C8 cycloalkylene, each substituted or unsubstituted heterocycloalkylene is a substituted or unsubstituted 3 to 8 membered heterocycloalkylene, each substituted or unsubstituted arylene is a substituted or unsubstituted C6-C10 arylene, and/or each substituted or unsubstituted heteroarylene is a substituted or unsubstituted 5 to 10 membered heteroarylene.

In some embodiments, each substituted or unsubstituted alkyl is a substituted or unsubstituted C1-C8 alkyl, each substituted or unsubstituted heteroalkyl is a substituted or unsubstituted 2 to 8 membered heteroalkyl, each substituted or unsubstituted cycloalkyl is a substituted or unsubstituted C3-C7 cycloalkyl, each substituted or unsubstituted heterocycloalkyl is a substituted or unsubstituted 3 to 7 membered heterocycloalkyl, each substituted or unsubstituted aryl is a substituted or unsubstituted C6-C10 aryl, and/or each substituted or unsubstituted heteroaryl is a substituted or unsubstituted 5 to 9 membered heteroaryl. In some embodiments, each substituted or unsubstituted alkylene is a substituted or unsubstituted C1-C8 alkylene, each substituted or unsubstituted heteroalkylene is a substituted or unsubstituted 2 to 8 membered heteroalkylene, each substituted or unsubstituted cycloalkylene is a substituted or unsubstituted C3-C7 cycloalkylene, each substituted or unsubstituted heterocycloalkylene is a substituted or unsubstituted 3 to 7 membered heterocycloalkylene, each substituted or unsubstituted arylene is a substituted or unsubstituted C6-C10 arylene, and/or each substituted or unsubstituted heteroarylene is a substituted or unsubstituted 5 to 9 membered heteroarylene.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of cancer.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “cancer” refers to all types of cancer, neoplasm or malignant tumors found in mammals, including leukemias, lymphomas, melanomas, neuroendocrine tumors, carcinomas and sarcomas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma encuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma exulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

“Chemotherapeutic” or “chemotherapeutic agent” is used in accordance with its plain ordinary meaning and refers to a chemical composition or compound having antineoplastic properties or the ability to inhibit the growth or proliferation of cells.

### 2. Meditopes

Provided herein are meditopes for use in preparing the antibody-meditope conjugates described herein. The meditopes are peptides which contain a photoreactive functional group capable of forming a covalent bond upon irradiation with UV light. In general, the term “meditope,” as used herein, refers to a peptide or peptides which is capable of binding to a central cavity, such as a meditope-binding site of a meditope-enabled antibody or antigen binding fragment thereof, as disclosed herein. In some embodiments, the meditope sequence is cyclized as disclosed herein (e.g. the terminal (or near terminal) cysteine residues form a disulfide bond).

Among the provided meditopes are meditope variants (also called variant meditopes), having one or more modifications, e.g., structural modifications, as compared a meditope of SEQ ID NO: 1 or 2), and methods for producing the same. In some embodiments, cQFD and cQYN meditopes are used as starting points in the design of meditope variants. In some aspects, the meditope variants are designed to have altered properties, such as increased or altered affinity, altered pH dependence, or different affinities under different physiological conditions for one or more of the provided meditope-enabled antibodies, including cetuximab and other antibodies described herein, e.g., as compared to the unmodified meditopes, cQFD and cQYN. Meditope variants are designed and produced using various chemical and biophysical methods.

Meditope variants include, but are not limited to, variants incorporating modifications to meditopes, e.g., cQFD and cQYN and others described herein. Suitable modifications include, but are not limited to, any peptide modification known in the art, such as, but not limited to, modifications to the manner and/or position of peptide cyclization, modifications to one or more amino acid components of the cyclic peptide, or adding or deleting one or more amino acid from the cyclic peptide. In a particular example, cQFD may be altered with one or more of the following modifications: a modification of Arg8, a modification of Phe3, a modification of Leu5, a modification of Leu10, change to the mode of peptide cyclization, and/or an incorporation of hydratable carbonyl functionality at one or more positions, and one or more amino acid deletions or additions. In the case of cQYN, suitable modifications may include one or more of the following: a modification of Arg8, a modification of Leu5, a modification of Leu10, change to the mode of peptide cyclization, and/or an incorporation of hydratable carbonyl functionality at one or more positions, and one or more deletions or additions. Certain amino acid positions within the meditope may be deleted or replaced with a different natural amino acid or an unnatural amino acid, or the meditope may be chemically conjugated with a fragment, for example by using “click chemistry.” In addition, the amino and carboxy termini can be extended with further amino acids beyond (i.e., in addition to) the cyclic portion of the meditope variant in order to make additional contact to the Fab.

Also provided herein is a peptide comprising the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Phe3, Asp4, Arg8, Arg9, and/or Leu10, and further wherein the modification comprises incorporation of a photoreactive functional group capable of forming a covalent bond upon irradiation with UV light. Also provided herein is a peptide comprising the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Asp4, Leu5, Arg8, Arg9, and/or Leu10, and further wherein the modification comprises incorporation of a photoreactive functional group capable of forming a covalent bond upon irradiation with UV light. In one embodiment, the amino acid sequence comprises a modification at Phe3. In one embodiment, the amino acid sequence comprises a modification at Asp4. In one embodiment, the amino acid sequence comprises a modification at Leu5. In one embodiment, the amino acid sequence comprises a modification at Arg8. In one embodiment, the amino acid sequence comprises a modification at Arg9. In one embodiment, the amino acid sequence comprises a modification at Leu10.

Provided herein is a peptide comprising the amino acid sequence of CQYNLSSRALKC (SEQ ID NO: 2) or SEQ ID NO: 2 with one or two amino acid additions, deletions and/or substitutions, wherein the amino acid sequence comprises one or more modifications of at least one amino acid residue selected from the group consisting of Tyr3, Asn4, Leu5, Arg8, Ala9, and/or Leu10, and further wherein the modification comprises incorporation of a photoreactive functional group capable of forming a covalent bond upon irradiation with UV light. In one embodiment, the amino acid sequence comprises a modification at Tyr3. In one embodiment, the amino acid sequence comprises a modification at Asn4. In one embodiment, the amino acid sequence comprises a modification at Leu5. In one embodiment, the amino acid sequence comprises a modification at Arg8. In one embodiment, the amino acid sequence comprises a modification at Ala9. In one embodiment, the amino acid sequence comprises a modification at Leu10.

These peptides, also referred to herein as “meditopes” bind to a central cavity such as a meditope-binding site or a meditope-enabled cavity of a meditope-enabled antibody or antigen binding fragment thereof. The binding can be to one or more functional groups on the backbone or side chains of amino acids within the meditope-binding site or meditope-enabled cavity. In certain embodiments, the antibody or antigen binding fragment thereof has a threonine at position 40, an asparagine at position 41, and/or an aspartate at position 85 of its light chain, according to Kabat numbering, or contains a meditope-binding site containing residues that correspond to those within the meditope-binding site of cetuximab, meditope-enabled trastuzumab, or meditope-enabled M5A, disclosed herein.

In general, the meditope can be modified at one or more position, such that its ability to effectively non-covalently bind to the meditope-enabled cavity is not significantly diminished. In addition, in certain embodiments, the meditope may be modified by adding, deleting or substituting one or more amino acids, to enhance the non-covalent bind to the meditope-enabled cavity. Accordingly, in one embodiment, the meditopes have an amino acid sequence length of between 5 and 16 amino acids, e.g., 5, 6, 7, 8, 9, 10, 11, 12, 13, 14, 15, or 16 amino acids in length, such as between 8 and 13 amino acids in length, e.g., between 9 and 12 amino acids in length, provided that the amino acid sequence comprises one or more modifications of at least one amino acid residue at a position selected from the group consisting of 3, 4, 5, 8, 9, and/or 10, and further wherein the modification comprises incorporation of a photoreactive functional group capable of forming a covalent bond upon irradiation with UV light.

In some embodiments, the variant meditopes are cyclic peptides. In other embodiments, they are linear, or acyclic, peptides.

The meditopes can include peptides, or cyclic peptides derived from such peptides, for example, where the peptides have the formula:

X1-X2-X3-X4-X5-X6-X7-X8-X9-X10-X11-X12

where:

X1=Cys, Gly, β-alanine, diaminopropionic acid, β-azidoalanine, or null;

X2=Gln or null;

X3=Phe, Tyr, β-β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;

X4=Asp or Asn;

X5=Leu, β-β′-diphenyl-Ala, Phe, Trp, Tyr, a non-natural analog of phenylalanine, a hydratable carbonyl-containing residue, or a boronic acid-containing residue;

X6=Ser;

X7=Thr or Ser;

X8=Arg, Ser, a modified Arg, or a hydratable carbonyl-containing residue, or boronic acid-containing residue;

X9=Arg or Ala;

X10=Leu, Gln, Glu, β-β′-diphenyl-Ala, Phe, Trp, Tyr, a non-natural analog of phenylalanine, a hydratable carbonyl-containing residue, or a boronic acid-containing residue;

X11=Lys or Arg; and

X12=Cys, Gly, 7-aminoheptanoic acid, β-alanine, diaminopropionic acid, propargylglycine, aspartic acid, isoaspartic acid, or null;

provided that the amino acid sequence comprises one or more modifications of at least one amino acid residue at a position selected from the group consisting of X3, X4, X5, X8, X9, and/or X10, and further wherein the modification comprises incorporation of a photoreactive functional group capable of forming a covalent bond upon irradiation with UV light.

In certain embodiments, the peptide is selected from the group consisting of:

(SEQ ID NO: 3)
CQX¹DLSTRRLRC,
(SEQ ID NO: 4)
CQFX¹LSTRRLRC,
(SEQ ID NO: 5)
CQFDLSTX¹RLRC,
(SEQ ID NO: 6)
CQFDLSTRX¹LRC,
and
(SEQ ID NO: 7)
CQFDLSTRRX¹RC,

or a peptide of SEQ ID NOs: 3-7 with one or two amino acid additions, deletions and/or substitutions, wherein X¹ is an unnatural amino acid comprising a photoreactive functional group capable of forming a covalent bond upon irradiation with UV light.

In certain embodiments, the peptide is selected from the group consisting of:

(SEQ ID NO: 8)
CQX¹NLSSRALKC,
(SEQ ID NO: 9)
CQYX¹LSSRALKC,
(SEQ ID NO: 10)
CQYNX¹SSRALKC,
(SEQ ID NO: 11)
CQYNLSSX¹ALKC,
(SEQ ID NO: 12)
CQYNLSSRX¹LKC,
and
(SEQ ID NO: 13)
CQYNLSSRAX¹KC,

or a peptide of SEQ ID NOs: 8-13 with one or two amino acid additions, deletions and/or substitutions, wherein X¹ is an unnatural amino acid comprising a photoreactive functional group capable of forming a covalent bond upon irradiation with UV light.

The peptide (or meditope) can be linear or cyclized as disclosed herein (e.g. the cysteine residues form a disulfide bond). The peptide may also comprise one or more modifications selected from one or more of head-to tail lactam cyclic peptides, a linear peptide, an incorporation of an unnatural amino acid, a shortening or lengthening of a bond, or an incorporation of hydratable carbonyl functionality.

In some embodiments, the meditopes are peptides having the structure of Formula (I):

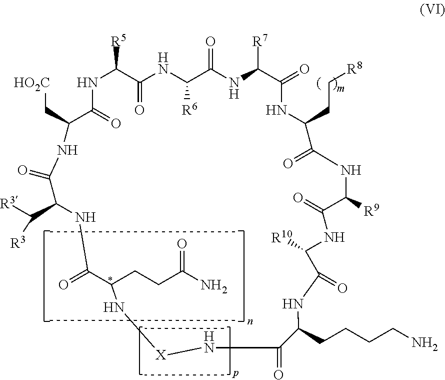

wherein:
the center marked with “*” is in the “R” or “S” configuration;
R³ and R^{3′} are independently H or phenyl, optionally substituted with one, two, or three substituents independently selected from C₁₋₄alkyl, —OH, fluoro, chloro, bromo, iodo and a photoreactive functional group;
R⁵ is:
   (A) C₁₋₈alkyl, optionally substituted with one or more substituents selected from oxo, acetal, ketal, —B(OH)₂, boronic ester, phosphonate ester, ortho ester, —CO₂C₁₋₄alkyl, —CH═CH—CHO, —CH═CH—C(O)C₁₋₄alkyl, —CH═CH—CO₂C₁₋₄alkyl, —CO₂H, —CONH₂ and a photoreactive functional group; or
   (B) C₁₋₄alkyl substituted with:
      a) one or two phenyl, wherein each phenyl is optionally substituted with one, two, or three substituents independently selected from —OH, fluoro, chloro, bromo, iodo and a photoreactive functional group; or
      b) naphthyl, imidazole, or indole;
R⁶ is —C₁₋₄alkylene-OH or —C₁₋₄alkylene-SH;
R⁷ is —C₁₋₄alkylene-OH or —C₁₋₄alkylene-SH;
m is 0, 1, 2, 3, 4, or 5;
R⁸ is:
   (a) —OH, —NR^{a}R^{b}, —N(RC)C(O)R, or —N(R)C(═NR^{d})R^{e};
   wherein:
      R^{a} is H;
      R^{b} is H or C₁₋₈alkyl optionally substituted with one or more substituents selected from the group consisting of oxo, acetal, ketal, —B(OH)₂, —SH, boronic ester, phosphonate ester, ortho ester, —CH═CH—CHO, —CH═CH—C(O)C₁₋₄alkyl, —CH═CH—CO₂C₁₋₄alkyl, —CO₂H, and —CO₂C₁₋₄alkyl;
      R^{c} is H, C₁₋₈alkyl, C₃₋₈cycloalkyl, branched alkyl, or aryl;
      R^{d} is H or a C₁₋₈alkyl, C₂₋₈alkenyl, C₂₋₈alkynyl, C₃₋₈cycloalkyl, branched alkyl, or aryl group, each optionally substituted with one or more substituents selected from the group consisting of —N₃, —NH₂, —OH, —SH, halogen, oxo, acetal, ketal, —B(OH)₂, boronic ester, phosphonate ester, ortho ester, —CH═CH—CHO, —CH═CH—C(O)C₁₋₄ alkyl, —CH═CH—CO₂C₁₋₄alkyl, —CO₂H, and —CO₂C₁₋₄alkyl group;
      R^{e} is H, —NHR^{d}, or a C₁₋₁₂alkyl, C₃₋₈cycloalkyl, C₂₋₁₂alkenyl, C₂₋₈alkynyl, or aryl group, each optionally substituted with one or more substituents selected from the group consisting of —N₃, —NH₂, —OH, —SH, oxo, C₂₋₄acetal, C₂₋₄ketal, —B(OH)₂, boronic ester, phosphonate ester, ortho ester, —CH═CH—CHO, —CH═CH—C(O)C₁₋₄alkyl, —CH═CH—CO₂C₁₋₄alkyl, and —CO₂C₁₋₄alkyl; or
   (b) C₁₋₁₂ alkyl substituted with an oxo, acetal, ketal, —B(OH)₂, boronic ester, —SH, —OH, phosphonate ester, ortho ester, —CH═CH—CHO, —CH═CH—C(O)C₁₋₄alkyl, —CH═CH—CO₂C₁₋₄alkyl, —CO₂C₁₋₄alkyl or a photoreactive functional group;
R⁹ is C₁₋₄alkyl, —C₁₋₂alkylene-CO₂H, —C₁₋₂alkylene-CONH₂, —C₁₋₂alkylene-CH₂NHC(O)NH₂, —C₁₋₂alkylene-CH₂NHC(═NH)NH₂;
   wherein R^{x} is a photoreactive functional group;
R¹⁰ is:
   (1) C₁₋₈alkyl optionally substituted with one or more substituents selected from the group consisting of oxo, acetal, ketal, —B(OH)₂, boronic ester, phosphonate ester, ortho ester, —CH═CH—CHO, —CH═CH—C(O)C₁₋₄alkyl, —CH═CH—CO₂C₁₋₄alkyl, —CO₂C₁₋₄alkyl, —CO₂H, —CONH₂ and a photoreactive functional group; or
   (2) C₁₋₄alkyl substituted with one or two phenyl, or one naphthyl, imidazole, or indole, wherein each phenyl is optionally substituted with one, two, or three substituents independently selected from —OH, fluoro, chloro, bromo, iodo and a photoreactive functional group;
n is 0 or 1;
p is 0 or 1;
X is C₁₋₈alkylene or C₂₋₈alkenylene, each carbon thereof optionally substituted with oxo, —C(O)—, —NHC(O)—, —CO₂H, —NH₂, or —NHC(O)R^{y};
   wherein one carbon of said alkylene is optionally replaced with —C(O)NH—, a 5-membered heteroaryl ring, or —S—S—; and
   R^{y} is —C₁₋₄alkyl, —CH(R^{z})C(O)— or —CH(R^{z})CO₂H;
      R^{z} is —H or —C₁₋₄alkyl optionally substituted with —OH, —SH, or —NH₂;
         or a pharmaceutically acceptable salt thereof,

provided that at least one of R³, R^{3′}, R⁵, R⁸, R⁹, and/or R¹⁰ comprises a photoreactive functional group capable of forming a covalent bond upon irradiation with UV light.

The center marked with “*” is in the “R” or “S” configuration. The symbol [symbol] denotes the point of attachment of R^{1A} to L^{1A}.

In certain embodiments, R³ and R^{3′} are each, independently, H or phenyl, optionally substituted with one, two, or three substituents independently selected from C₁₋₄alkyl, —OH, fluoro, chloro, bromo, and iodo.

In certain embodiments, R⁵ is: (A) C₁₋₈alkyl, optionally substituted with one or more substituents selected from oxo, acetal, ketal, —B(OH)₂, boronic ester, phosphonate ester, ortho ester, —CO₂C₁₋₄alkyl, —CH═CH—CHO, —CH═CH—C(O)C₁₋₄alkyl, —CH═CH—CO₂C₁₋₄alkyl, —CO₂H, and —CONH₂; or (B) C₁₋₄alkyl substituted with: a) one or two phenyl groups, wherein each phenyl is optionally substituted with one, two, or three substituents independently selected from —OH, fluoro, chloro, bromo, and iodo; or b) a naphthyl, imidazole, or indole group.

In certain embodiments, R⁶ is —C₁₋₄alkylene-OH or —C₁₋₄alkylene-SH. R⁷ is —C₁₋₄ alkylene-OH or —C₁₋₄alkylene-SH. The symbol m is 0, 1, 2, 3, 4, or 5.

In certain embodiments, R⁸ is —OH, —NR^{a}R^{b}, —N(R)C(O)R^{e}, or —N(R)C(═NR^{d})R^{e}. R^{a} is H. R^{b} is H or C₁₋₈alkyl optionally substituted with one or more substituents selected from oxo, acetal, and ketal, —B(OH)₂, —SH, boronic ester, phosphonate ester, ortho ester, —CH═CH—CHO, —CH═CH—C(O)C₁₋₄alkyl, —CH═CH—CO₂C₁₋₄alkyl, —CO₂H, or —CO₂C₁₋₄alkyl. R^{c} is H, C₁₋₈alkyl, C₃₋₈cycloalkyl, branched alkyl, or aryl. R^{d} is H or a C₁₋₈alkyl, C₂₋₈alkenyl, C₂₋₈alkynyl, C₃₋₈cycloalkyl, branched alkyl, or aryl, each optionally substituted with one or more substituents selected from —N₃, —NH₂, —OH, —SH, halogen, oxo, acetal, ketal, —B(OH)₂, boronic ester, phosphonate ester, ortho ester, —CH═CH—CHO, —CH═CH—C(O)C₁₋₄alkyl, —CH═CH—CO₂C₁₋₄alkyl, —CO₂H, and —CO₂C₁₋₄alkyl group. R^{e} is H; —NHR^{d}; or a C₁₋₁₂alkyl, C₃₋₈cycloalkyl, C₂₋₁₂alkenyl, C₂₋₈alkynyl, or aryl group, each optionally substituted with one or more substituents selected from —N₃, —NH₂, —OH, —SH, oxo, C₂₋₄acetal, C₂₋₄ketal, —B(OH)₂, boronic ester, phosphonate ester, ortho ester, —CH═CH—CHO, —CH═CH—C(O)C₁₋₄alkyl, —CH═CH—CO₂C₁₋₄alkyl, and —CO₂C₁₋₄alkyl. Alternatively, R⁸ is a C₁₋₁₂ alkyl substituted with an oxo, acetal, ketal, —B(OH)₂, boronic ester, —SH, —OH, phosphonate ester, ortho ester, —CH═CH—CHO, —CH═CH—C(O)C₁₋₄alkyl, —CH═CH—CO₂C₁₋₄alkyl, or —CO₂C₁₋₄alkyl.

In certain embodiments, R⁹ is C₁₋₄alkyl or —C₁₋₂alkylene-CO₂H, —C₁₋₂alkylene-CONH₂, —C₁₋₂alkylene-CH₂NHC(O)NH₂, or —C₁₋₂alkylene-CH₂NHC(═NH)NH₂.

In certain embodiments, R¹⁰ is: (1) C₁₋₈alkyl optionally substituted with one or more substituents selected from oxo, acetal, ketal, —B(OH)₂, boronic ester, phosphonate ester, ortho ester, —CH═CH—CHO, —CH═CH—C(O)C₁₋₄alkyl, —CH═CH—CO₂C₁₋₄alkyl, —CO₂C₁₋₄alkyl, —CO₂H, and —CONH₂; or (2) C₁₋₄alkyl group substituted with one or two phenyl groups, or one naphthyl, imidazole, or indole group, wherein each phenyl is optionally substituted with one, two, or three substituents independently selected from —OH, fluoro, chloro, bromo, and iodo.

In certain embodiments, n is 0 or 1. In certain embodiments, p is 0 or 1.

In certain embodiments, X is (1) a linker resulting from any of the meditope cyclization strategies discussed herein; (2) substituted alkylene, substituted heteroalkylene, substituted cycloalkylene, substituted heterocycloalkylene, substituted arylene or substituted heteroarylene or (3) C₁₋₈alkylene or C₂₋₈alkenylene, each carbon thereof optionally substituted with oxo, —C(O)—, —NH₂, —NHC(O)— or —NHC(O)R. One carbon of the X C₁₋₈alkylene is optionally replaced with —C(O)NH—, a 5-membered heteroaryl ring, or —S—S—. R^{y} is —C₁₋₄alkyl or —CH(R^{z})C(O)— or —CH(R_{z})CO₂H. R^{z} is —H or —C₁₋₄alkyl optionally substituted with —OH, —SH, or —NH₂. Formula VII includes all appropriate pharmaceutically acceptable salts. In (1), X is considered a substituted linker due to its chemical trivalency and because X may optionally include further substituents as set forth above (e.g. —NH₂ and oxo). In some embodiments, X is:

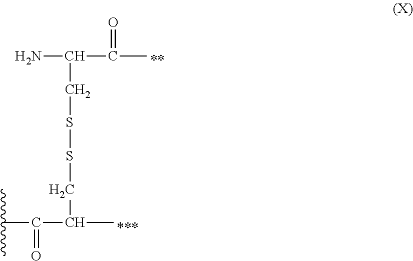 [SMILES: *C(=O)C(C)CSSCC(N)C(C)=O]

where ** represents the point of attachment to the glutamine attached to X in Formula VII and *** represents the point of attachment to the nitrogen attached to X and lysine in Formula VII. The symbol [symbol] denotes the point of attachment of X to the remainder of the molecule.

In some embodiments of the meditope of Formula VII, m is 0, 1, or 2. In other embodiments, R³ is H or phenyl and R^{3′} is phenyl, 2-bromophenyl, 3-bromophenyl, or 4-bromophenyl. In further embodiments, R⁵ is methyl, ethyl, propyl, isopropyl, butyl, isobutyl, sec-butyl, or tert-butyl, each optionally substituted with an oxo, —B(OH)₂, —CO₂H, or —CONH₂ group, or with one or two phenyl groups each optionally substituted with a bromo or chloro substituent. In further embodiments, R⁸ is —OH, —NH₂, —N(R^{c})C(O)R^{e}, or —N(R^{c})C(═NR^{d})R^{e}. In still further embodiments, R^{c} is H or methyl, R^{d} is H or C₁₋₄alkyl, and R^{e} is C₁₋₄alkyl, or —NH(C₁₋₄alkyl). In other embodiments, R⁹ is methyl or ethyl, optionally substituted with —CO₂H, —CONH₂, —CH₂NHC(O)NH₂, or —CH₂NHC(═NH)NH₂. In still other embodiments, R¹⁰ is methyl, ethyl, propyl, isopropyl, butyl, isobutyl, sec-butyl, or tert-butyl, each optionally substituted with an oxo, —B(OH)₂, —CO₂H, or —CONH₂ group. In still other embodiments, —X—NH— is -Cys-Cys- (e.g. bound through a disulfide bridge), -Gly-Gly-, —C(O)(CH₂)₆—NH—, -β-Ala-β-Ala-, —C(O)CH(NH₂)CH₂CH═CHCH₂CH(CO₂H)—NH—, —C(O)CH(NH₂)CH₂NHC(O)CH₂CH(CO₂H)—NH—, -β-Ala-C(O)CH₂CH(CO₂H)—NH—, or —C(O)CH(NH₂)CH₂-triazinyl-CH₂—CH(CO₂H)—NH—.

In certain embodiments, the peptide is a compound of Formula IA:

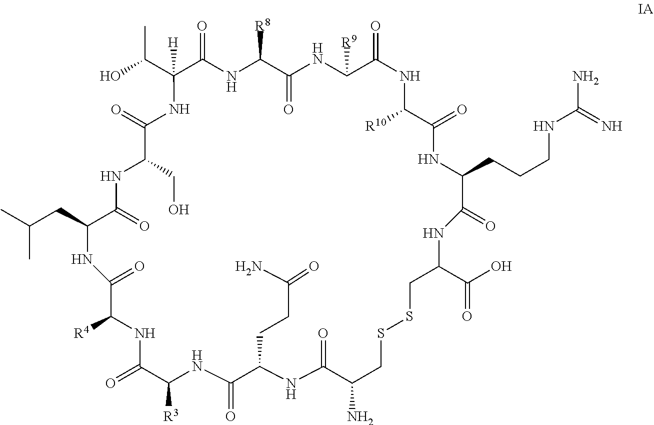

wherein each of R³, R⁴, R⁸, R⁹ and R¹⁰ is independently -Lₙ-alkyl-R^{x}, -Lₙ-cycloalkyl-R^{x}, -Lₙ-aryl-R^{x}, -Lₙ-heterocyclyl-R^{x}, or -Lₙ-heteroaryl-R^{x},

R^{x} is a photoreactive functional group capable of forming a covalent bond upon irradiation with UV light;

each L is independently alkylene, cycloalkylene, arylene, heterocyclylene or heteroarylene;

each n is independently 0, 1 or 2; and

each alkyl, cycloalkyl, aryl, heterocyclyl, heteroaryl, alkylene, cycloalkylene, arylene, heterocyclylene and heteroarylene is substituted or unsubstituted.

In certain embodiments, the peptide is a compound of Formula IB:

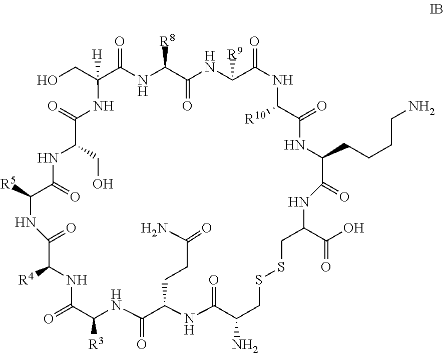

wherein each of R³, R⁴, R⁵, R⁸, R⁹ and R¹⁰ is independently -Lₙ-alkyl-R^{x}, -Lₙ-cycloalkyl-R^{x}, -Lₙ-aryl-R^{x}, -Lₙ-heterocyclyl-R^{x}, or -Lₙ-heteroaryl-R^{x};

R^{x} is a photoreactive functional group capable of forming a covalent bond upon irradiation with UV light;

each L is independently alkylene, cycloalkylene, arylene, heterocyclylene or heteroarylene;

each n is independently 0, 1 or 2; and

each alkyl, cycloalkyl, aryl, heterocyclyl, heteroaryl, alkylene, cycloalkylene, arylene, heterocyclylene and heteroarylene is substituted or unsubstituted.

Also provided herein is a peptide comprising the amino acid sequence of CQFDLSTRRX¹RC (SEQ ID NO: 7) or SEQ ID NO: 7 with one or two amino acid additions, deletions and/or substitutions, wherein X¹ comprises a photoreactive functional group capable of forming a covalent bond upon irradiation with UV light. The peptide can be linear or cyclized (e.g. the cysteine residues form a disulfide bond). Accordingly, in certain embodiments, the peptide comprises a compound of Formula IIA:

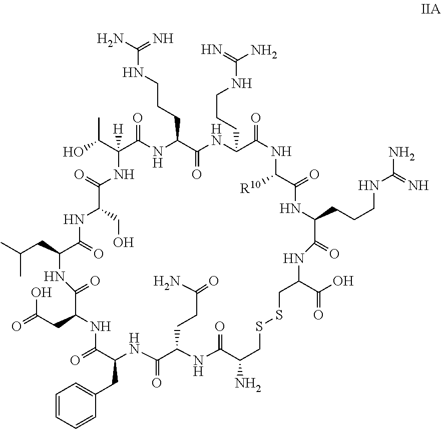

wherein R¹⁰ is -Lₙ-alkyl-R^{x}, -Lₙ-cycloalkyl-R^{x}, -Lₙ-aryl-R^{x}, -Lₙ-heterocyclyl-R^{x}, or -Lₙ-heteroaryl-R^{x};

R^{x} is a photoreactive functional group capable of forming a covalent bond upon irradiation with UV light;

each L is independently alkylene, cycloalkylene, arylene, heterocyclylene or heteroarylene;

each n is independently 0, 1 or 2; and

each alkyl, cycloalkyl, aryl, heterocyclyl, heteroaryl, alkylene, cycloalkylene, arylene, heterocyclylene and heteroarylene is substituted or unsubstituted.

Also provided herein is a peptide comprising the amino acid sequence of CQYNLSSRAX¹KC (SEQ ID NO: 13) or SEQ ID NO: 13 with one or two amino acid additions, deletions and/or substitutions, wherein X comprises a photoreactive functional group capable of forming a covalent bond upon irradiation with UV light. The peptide can be linear or cyclized (e.g. the cysteine residues form a disulfide bond). Accordingly, in certain embodiments, the peptide comprises a compound of Formula IIB:

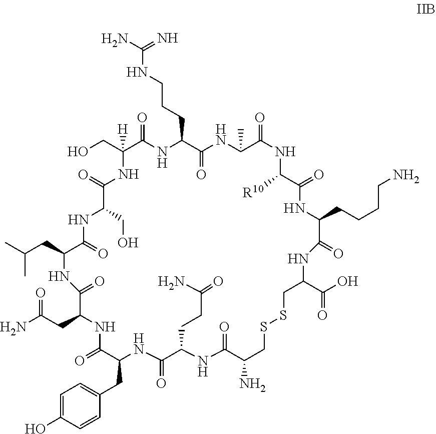

wherein R¹⁰ is -Lₙ-alkyl-R^{x}, -Lₙ-cycloalkyl-R^{x}, -Lₙ-aryl-R^{x}, -Lₙ-heterocyclyl-R^{x}, or -Lₙ-heteroaryl-R^{x};

R^{x} is a photoreactive functional group capable of forming a covalent bond upon irradiation with UV light;

each L is independently alkylene, cycloalkylene, arylene, heterocyclylene or heteroarylene;

each n is independently 0, 1 or 2; and

each alkyl, cycloalkyl, aryl, heterocyclyl, heteroaryl, alkylene, cycloalkylene, arylene, heterocyclylene and heteroarylene is substituted or unsubstituted.

In any one of the above embodiments, the photoreactive functional group (R^{x}) is a side chain of a natural or unnatural amino acid which comprises at least one functional group which forms a reactive species upon irradiation with UV light, and thus is capable of forming a covalent bond. Exemplary functional groups include, but are not limited to, an azide (e.g., aryl azides, azido-methyl-coumarins, and the like), benzophenones, anthraquinones, diazo compounds, diazirines, and psoralen derivatives. In certain embodiments, the photoreactive functional group (e.g., R^{x}) comprises a benzophenone, an azide or a diazirine.

In one embodiment, the peptide comprises a compound of Formula IA or IB, and each of R³, R⁴, R⁵, R⁸, R⁹ and R¹⁰ is independently:

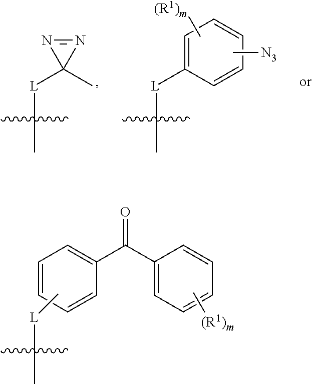

wherein each L is independently alkylene, cycloalkylene, arylene, heterocyclylene or heteroarylene;

each R¹ is independently halo, hydroxyl, alkyl, or nitro;

m is 0, 1, 2, 3 or 4; and

each alkylene, cycloalkylene, arylene, heterocyclylene and heteroarylene is substituted or unsubstituted.

In one embodiment, the peptide comprises a compound of Formula IA or IB, and each of R³, R⁴, R⁵, R⁸, R⁹ and R¹⁰ is independently:

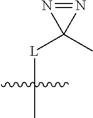

wherein L is alkylene, cycloalkylene, arylene, heterocyclylene or heteroarylene; and

each alkylene, cycloalkylene, arylene, heterocyclylene and heteroarylene is substituted or unsubstituted.

In one embodiment, the peptide comprises a compound of Formula IA, IB, IIA or IIB, and R¹⁰ is:

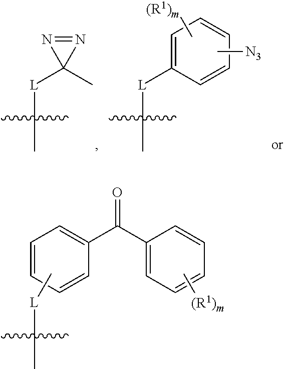

wherein each L is independently alkylene, cycloalkylene, arylene, heterocyclylene or heteroarylene;

each R¹ is independently halo, hydroxyl, alkyl, or nitro;

m is 0, 1, 2, 3 or 4; and

each alkylene, cycloalkylene, arylene, heterocyclylene and heteroarylene is substituted or unsubstituted.

In one embodiment, the peptide comprises a compound of Formula IA, IB, IIA or IIB, and R¹⁰ is:

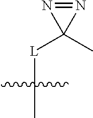

wherein L is alkylene, cycloalkylene, arylene, heterocyclylene or heteroarylene; and

each alkylene, cycloalkylene, arylene, heterocyclylene and heteroarylene is substituted or unsubstituted.

In certain embodiments, L is alkylene. In another embodiment, L is cycloalkylene. In another embodiment, L is arylene. In another embodiment, L is heterocyclylene. In another embodiment, L is heteroarylene.

In one embodiment, the peptide comprises a compound of Formula IA, IB, IIA or IIB, and R¹⁰ is:

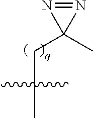

wherein q is 1, 2, 3, 4, 5 or 6.

In one embodiment, the peptide comprises a compound of Formula IIIA:

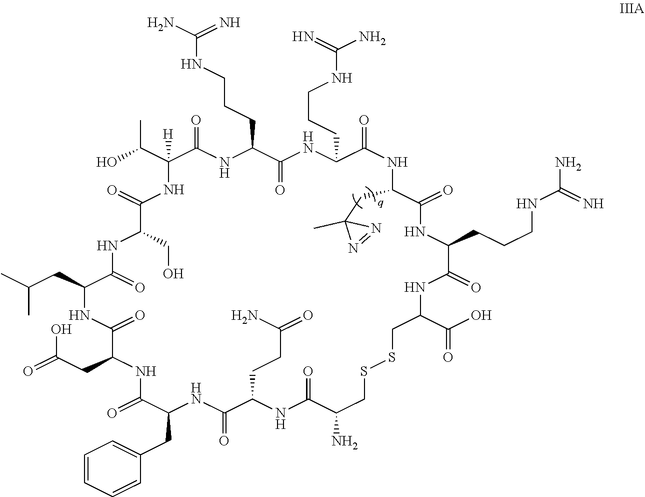

wherein q is 1, 2, 3, 4, 5 or 6.

In one embodiment, the peptide comprises a compound of Formula IIB:

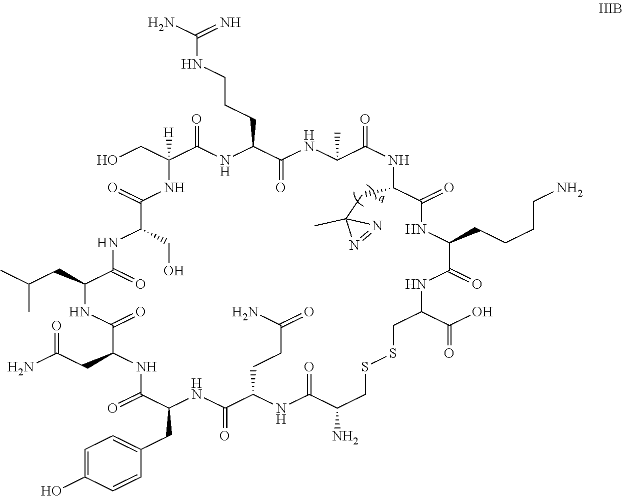

wherein q is 1, 2, 3, 4, 5 or 6.

In one embodiment, the peptide comprises a compound of Formula IA, IB, IIA, IIB, IIIA or IIIB, and R¹⁰ is:

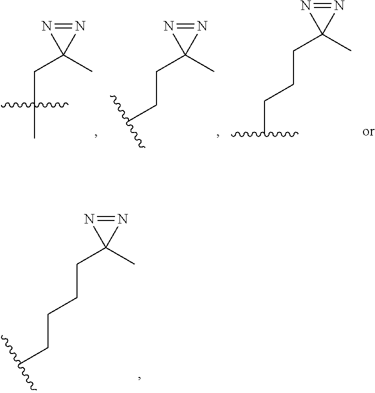

The peptides (i.e., meditopes) described herein provide a site specific option for linking an active agent (e.g., small molecules, proteins, etc.) to antibodies which have a meditope-binding site. One of the advantages of the meditopes disclosed herein, is that they are capable of covalently binding to antibodies, where the normal antibody function is protected and preserved even following attachment to the antibody. Since the covalent bond formation can be accomplished by applying long UV light for a short duration, potential damage to the antibody and/or an active agent is mitigated.

In certain embodiments, the meditope is conjugated to an agent, such as a therapeutic agent, a diagnostic agent, or a detectable agent. The peptide can be conjugated to the agent via any suitable means, such as via a chemical linker. In certain embodiments, the chemical linker is a covalent linker. In embodiments, the chemical linker comprises a substituted or unsubstituted alkylene, substituted or unsubstituted heteroalkylene, substituted or unsubstituted cycloalkylene, substituted or unsubstituted heterocycloalkylene, substituted or unsubstituted arylene and/or substituted or unsubstituted heteroarylene.

In certain embodiments, the chemical linker comprises a PEG linker. In certain embodiments, the chemical linker comprises a peptide linker (e.g., one or more natural or unnatural amino acids, such as glycine, lysine, tyrosine, glutamic acid, aspartic acid, etc.).

The chemical linker can be bonded to the meditope at any suitable location, such as the N-terminus, the C-terminus, and/or off a side chain. In certain embodiments, the chemical linker is bonded to the N-terminus. In another embodiment, the chemical linker is bonded to the C-terminus. In still another embodiment, the meditope comprises more than one chemical linker, where the chemical linkers are bonded to the N-terminus and the C-terminus. In some embodiments, the chemical linker comprises the amino acid sequence -GGGK.

Also provided herein is a peptide selected from the group consisting of:

(SEQ ID NO: 14)
CQX¹DLSTRRLRCGGGK,
(SEQ ID NO: 15)
CQFX¹LSTRRLRCGGGK,
(SEQ ID NO: 77)
CQFDX¹STRRLRCGGGK,
(SEQ ID NO: 16)
CQFDLSTX¹RLRCGGGK,
(SEQ ID NO: 17)
CQFDLSTRX¹LRCGGGK,
and
(SEQ ID NO: 18)
CQFDLSTRRX¹RCGGGK,

or a peptide of SEQ ID NOs: 14-18 with one or two amino acid additions, deletions and/or substitutions, wherein X¹ is an unnatural amino acid comprising a photoreactive functional group capable of forming a covalent bond upon irradiation with UV light. In certain embodiments, X is selected from the group consisting of L-2-amino-4,4-azi-pentanoic acid, L-2-amino-5,5-azi-hexanoic acid, azido-phenylalanine, and para-benzoylphenylalanine.

Also provided herein is a peptide selected from the group consisting of:

(SEQ ID NO: 19)
CQX¹NLSSRALKCGGGK,
(SEQ ID NO: 20)
CQYX¹LSSRALKCGGGK,
(SEQ ID NO: 21)
CQYNX¹SSRALKCGGGK,
(SEQ ID NO: 22)
CQYNLSSX¹ALKCGGGK,
(SEQ ID NO: 23)
CQYNLSSRX¹LKCGGGK,
and
(SEQ ID NO: 24)
CQYNLSSRAX¹KCGGGK,

or a peptide of SEQ ID NOs: 19-24 with one or two amino acid additions, deletions and/or substitutions, wherein X is an unnatural amino acid comprising a photoreactive functional group capable of forming a covalent bond upon irradiation with UV light. In certain embodiments, X¹ is selected from the group consisting of L-2-amino-4,4-azi-pentanoic acid, L-2-amino-5,5-azi-hexanoic acid, azido-phenylalanine, and para-benzoylphenylalanine.

In certain embodiments disclosed herein, the chemical linker may further comprise a commercially available linker known in the art for forming antibody-drug conjugates. Accordingly, in certain embodiments, the chemical linker may comprise a linker selected from the group consisting of Ala-Ala-Asn-PAB, ALD-BZ-OSu, ALD-di-EG-OPFP, ALD-di-EG-OSu, ALD-mono-EG-OPFP, ALD-mono-EG-OSu, ALD-tetra-EG-OPFP, ALD-tetra-EG-OSu, ALD-tri-EG-OPFP, ALD-tri-EG-OSu, BCOT-di-EG-OPFP, BCOT-di-EG-Osu, BCOT-tetra-EG-OPFP, BCOT-tetra-EG-OSu, BCOT-tri-EG-OPFP, BCOT-tri-EG-Osu, Boc-NMe-DAE, BrAH, Br-di-EG-OSu, Br-tetra-EG-OSu, Br-tri-EG-OSu, COT-acetic acid, COT-di-EG-OPFP, COT-di-EG-OSu, COT-tetra-EG-OPFP, COT-tetra-EG-OSu, COT-tri-EG-OPFP, COT-tri-EG-OSu, DHA, DHH, Fmoc-Ala-Ala-Asn-PAB-PNP, Fmoc-Phe-Lys(Trt)-PAB-PNP, Fmoc-Val-Cit-PAB, Fmoc-Val-Cit-PAB-PNP, HAC, MAH, MAL-di-EG-OPFP, MAL-di-EG-OSu, MAL-HA-OSu, MAL-tetra-EG-OPFP, MAL-tetra-EG-OSu, MAL-tri-EG-OPFP, MAL-tri-EG-OSu, MBA, MC-Val-Cit-PAB-PNP, MDB, MEL-di-EG-OPFP, MEL-di-EG-OSu, MEL-tetra-EG-OPFP, MEL-tetra-EG-OSu, MEL-tri-EG-OPFP, MEL-tri-EG-OSu, MMC, N₃BA, N3-di-EG-OPFP, N3-di-EG-OSu, N3-tetra-EG-OPFP, N3-tetra-EG-OSu, N3-tri-EG-OPFP, N3-tri-EG-OSu, PAB, PHA-di-EG-OPFP, PHA-di-EG-OSu, PHA-tetra-EG-OPFP, PHA-tetra-EG-OSu, PHA-tri-EG-OPFP, PHA-tri-EG-OSu, Phe-Lys(Fmoc)-PAB, Phe-Lys(Trt)-PAB, Py-ds-But-OPFP, Py-ds-But-OSu, Py-ds-dmBut-OPFP, Py-ds-dmBut-OSu, Py-ds-Prp-OPFP, Py-ds-Prp-OSu, and Val-Cit-PAB (ALB Materials Inc, Nevada, U.S.).

Non-limiting examples of peptides as disclosed herein, with or without toxin conjugation, are listed in the following Table 4.

TABLE 4

| Name | Description |
|---|---|
| Peptide 1 | L-Photo-Leucine (L-2-amino-4,4-azi-pentanoic acid) |
| | or (S)-2-amino-4- |
| | (3-methyl-3H-diazirin-3-yl)butanoic acid |
| Peptide 2 | L-Photo-Methionine (L-2-amino-5,5-azi-hexanoic |
| | acid) or (S)-2-amino-4- |
| | (3-methyl-3H-diazirin-3-yl)butanoic acid |
| Peptide 3 | (S)-2-amino-5-(3-methyl-3H-diazirin-3-yl)pentanoic acid |
| Peptide 4 | (S)-2-amino-6-(3-methyl-3H-diazirin-3-yl)hexanoic acid |
| Peptide 5 | MMAD conjugated photoactivatable |
| | long cQFD photo-Met 10 |
| Peptide 6 | MMAD conjugated photoactivatable long |
| | cQFD photo-Met 9 |
| Peptide 7 | MMAD conjugated photoactivatable long |
| | cQFD photo-Met 8 |
| Peptide 8 | Duostatin conjugated photoactivatable long |
| | cQFD photo-Met 10 |
| Peptide 9 | MMAD conjugated photoactivatable long |
| | cQFD azido-Phe 6 |
| Peptide 10 | MMAD conjugated photoactivatable long |
| | cQFD azido-Phe 3 |
| Peptide 11 | MMAD conjugated photoactivatable |
| | long cQFD azido-Phe 10 |
| Peptide 12 | MMAD conjugated photoactivatable |
| | long cQFD benzoyl-Phe 3 |
| Peptide 13 | MMAD conjugated photoactivatable long |
| | cQFD benzolyl-Phe 10 |
| Peptide 14 | Ac-K(Glu-PAB-vc-MMAD) GGGCQFDLSTRRLRC-OH |
| Peptide 15 | Ac-CQFDLSTRRXRCGGGK with maytansinoid; |
| | C:C═Disulfide; X═photoMet; Peptide 24 |
| | with maytansinoid |
| Peptide 16 | Ac-CQFDLSTRRXRCGGGK with duocarmycin; |
| | C:C═Disulfide; X═photoMet; Peptide |
| | 24 with duocarmycin |
| Peptide 17 | Ac-CQFDLSTRRXRCGGGK with PBD; C:C═Disulfide; |
| | X═photoMet;Peptide 24 with PBD toxin |
| Peptide 18 | Ac-CQFDLSTRRXRCGGGK with amanitin; |
| | C:C═Disulfide; X═photoMet; Peptide 24 with amanitin |
| Peptide 19 | Ac-CQFDLSTRRXRCGGGK with |
| | Duostatin; -Glu-vc-PAB-Duostatin-; |
| | where X═(S)-2-amino-5-(3-methyl-3H- |
| | diazirin-3-yl) pentanoic acid |
| Peptide 20 | Ac-CQFDLSTRRXRCGGGK with |
| | Duostatin; -Glu-vc-PAB-Duostatin-; |
| | where X═(S)-2-amino-6-(3-methyl-3H- |
| | diazirin-3-yl) hexanoic acid |
| Peptide 21 | Ac-KGGGCQFDLSTRRXRCGGGK-OH |
| | with Duostatin; N, C double |
| | long cQFD with photo-Met at position |
| | 10 with Duostatin (capable of |
| | bonding two active agents) |
| Peptide 22 | DM1 conjugated cQFD (cQFD-DM1) |
| Peptide 23 | MMAD conjugated cQFD (cQFD-MMAD) |
| Peptide 24 | long-cQFD-photoMet10 (4-carbon chain at position 10) |
| Peptide 25 | long-cQFD-photoMet10 (5-carbon chain at position 10) |
| Peptide 26 | Ac-CQFDLSTRRXRCGGGK-MCC-DM1 |
| | (X: 6-carbon chain (S)-2- |
| | amino-6-(3-methyl-3H-diazirin-3-yl)hexanoic |
| | acid at position 10) |

In one embodiment, Peptide 15 includes a payload maytaninoid emtansine (DM1) that is conjugated via maleimidomethyl cyclohexane-1-carboxylate (MCC) linker. The conjugated Peptide 15 has a structure as:

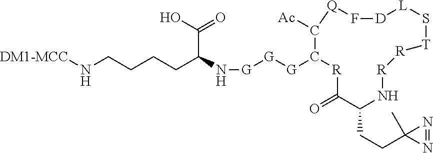

In another example, Peptide 16 includes a payload duocarmycin that is conjugated via a linker. A non-limiting example of the conjugated Peptide 16 is Ac-CQFDLSTRRXRCGGGK-PEG4-vc-PAB-duocarmycin SA with the following structure:

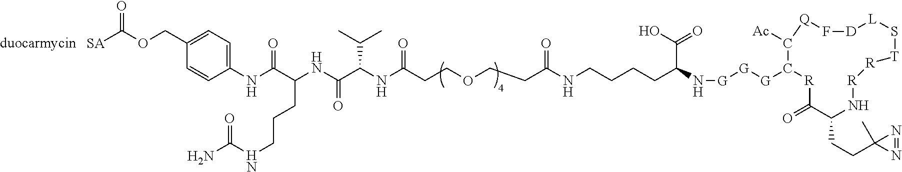

Another example is Peptide 17 that is conjugated with pyrrolobenzodiazepine (PBD). A non-limiting example of the conjugated Peptide 17 is Ac-CQFDLSTRRXRCGGGK-Glu-Val-Ala-PAB-PBD with the following structure:

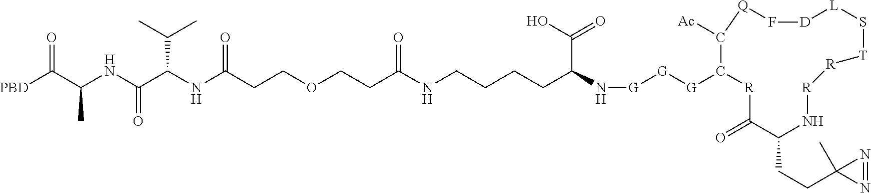

Another example is Peptide 18 conjugated with amanitin. A non-limiting example of the conjugated Peptide 18 is Ac-CQFDLSTRRXRCGGGK-linker-alpha-Amanitin with the following structure:

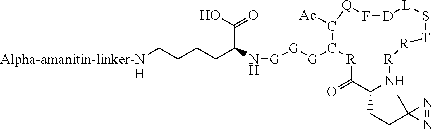

The disclosure herein also provides meditopes capable of bonding to more than one active agent (or toxin). These meditopes could be built by including more than one reactive functional moiety to any one of the meditopes (or peptides) as described herein, e.g., CQFDLSTRRLCGGGK (SEQ ID NO: 71). Non-limiting examples of such meditopes include Ac-KGGGCQFDLSTRRLCGGGK-OH (SEQ ID NO: 72), Ac-KGGGCQFDLSTRRLCGGGKGGKSGGAGK (SEQ ID NO: 73), Ac-CQFDLSTRRLCGGGKGGASGGAGKGGGK (SEQ ID NO: 74), Ac-CQFDLSTRRLCGGGKGGASGGGGSAGK (SEQ ID NO: 75), and Ac-KGGGCQFDLSTRRXRCGGGK-OH (SEQ ID NO: 76).

In certain embodiments, provided herein is an antibody-meditope conjugate, comprising a meditope and an antibody comprising a meditope-enabled cavity, wherein the side chain of amino acid X of the meditope is covalently bonded to the antibody, and wherein the meditope has more than one active agent bound thereto. In certain embodiments, the active agents are the same. In certain embodiments, the active agents are the different. One example of such a meditope is peptide 21, the structure of which is:

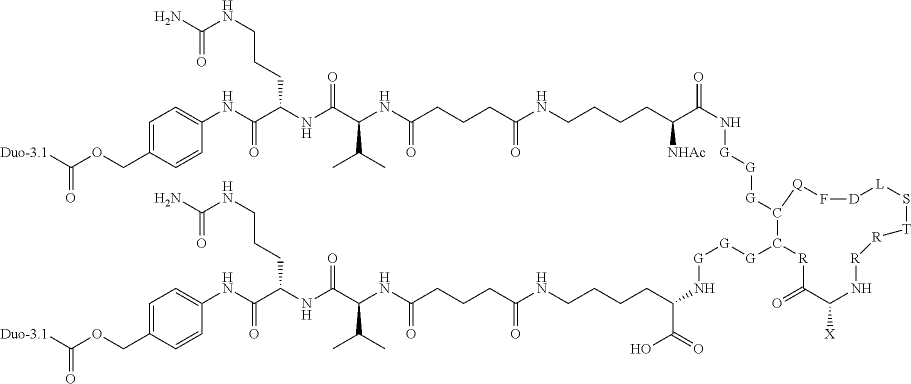

### 3. Antibody-Meditope Conjugates

Provided herein are antibody-meditope conjugates, comprising a cyclic meditope as described above and an antibody comprising a meditope-enabled cavity, wherein the meditope is covalently bonded to the antibody. In certain embodiments, the meditope comprises the amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Phe3, Asp4, Leu5, Arg8, Arg9, and/or Leu10, and wherein the modification comprises incorporation of a photoreactive functional group capable of forming a covalent bond upon irradiation with UV light.

It is believed that the covalent bond formation is enhanced by the tight and selective noncovalent association of the meditope to the antibody within the meditope-enabled cavity. It is further contemplated that certain positioning and/or conformation of the meditope in the meditope-enabled cavity facilitates the formation of the covalent bond or formation of a stable conjugate. In one such example, the meditope is positioned in the cavity such that the disulfide bridge between C1 and C12 of the meditope is closer to the entrance of the meditope-enabled cavity than the majority of amino acids 2-11 of the meditope such that the photoreactive functional group capable of forming a covalent bond is situated in a favorable position to allow efficient covalent bond formation to the antibody (see FIG. 5). In another example, the meditope peptide is positioned in such a way as to be closer to the hinge region of the heavy chain, and specific covalent interactions take place between amino acid 165 of the antibody and amino acid position 10 of the peptide. In another example, the peptide positioning is such that position 3 of the meditope peptide is curved upward and makes specific interactions with position 45 of the antibody to make an efficient covalent bond. In certain embodiments, the covalent bond between the antibody and the meditope is not, and does not contain, a disulfide bond.

It is contemplated that the covalent bond can form between the meditope and the backbone of the antibody and/or a side chain of an amino acid of the antibody. For instance, Leu10 or modified Leu10 may form a covalent bond with an amino acid at Kabat position 101 or 165 of the antibody. In another example, Phe3 or modified Phe3 forms a covalent bond with an amino acid at Kabat position 42 or 47 of the antibody. In certain embodiments, the meditope is covalently bound to the antibody via the side chain of an unnatural amino acid in the antibody. For example, the introduction on unnatural amino acids may be facilitated by unconventional amino-acyl tRNA synthetases that are capable of introducing noncanonical amino acids.

It is contemplated that by making one or more specific modifications to the meditope-enabled cavity of a meditope-enabled antibody, the binding affinity or specificity with respect to the photoreactive functional group on the meditope can be enhanced and this selective modification of the meditope-enabled cavity can result in enhanced binding efficiency. In one embodiment, residues that line the meditope-binding site of a meditope-enabled antibody and/or that are otherwise important for binding of such an antibody to a meditope, are systematically or randomly altered (e.g., using degenerate libraries and selection), for example, to enhance and/or change the specificity of the meditope (see, for example, Sheedy et al. 2007 and Akamatsu et al. 2007, hereby incorporated herein by reference, for methods of making alterations). In some aspects, the residues are substituted with natural or non-natural amino acids or both, in order to improve the affinity of the meditope interaction and/or alter another property of the meditope-antibody interaction. The incorporation of a non-natural amino acid in an antibody to generate a bi-specific antibody was described by Hutchins et al. 2011). Methods for providing modified antibodies are known (see, e.g., WO 2013/055404).

Residues of meditope-enabled antibodies that make contact with the meditope and/or are otherwise important, e.g., line the cavity, for example, residues within 8 Å of any atom of a bound meditope, that can be systematically or randomly changed to improve the meditope affinity through hydrogen bonding, ionic, electrostatic or steric interaction can include, but are not limited to, one or more light chain residues (e.g., P8, V9 or 19, 110 or L10, S14, E17, Q38, R39, T40, N41 G42, S43, P44, R45, D82, 183, A84, D85, Y86, Y87, G99, A100, G101, T102, K103, L104, E105, K107, R142, S162, V163, T164, E165, Q166, D 167, SI 68, or Y 173 of the light chain, based on Kabat numbering and with reference to cetuximab, meditope-enabled trastuzumab, or meditope-enabled M5A, or analogous residues in other meditope-enabled antibodies), and/or one or more heavy chain residues (e.g., Q6, P9, R38, Q39, S40, P41, G42, K43, G44, L45, S84, D86, T87, A88, 189, Y90, Y91, W103, G104, Q105, G106, T107, L108, V109, T110, V111, Y147, E150, P151, V152, T173, F174, P175, A176, V177, Y185, S186, or L187 of the heavy chain, based on Kabat numbering and with reference to cetuximab, meditope-enabled trastuzumab, or meditope-enabled M5A, or analogous residues in other meditope-enabled antibodies), or a combination thereof. It should be noted that unless otherwise specified, the numbering of the amino acid residues referred to with respect to the antibody are based on Kabat numbering.

For example, in some aspects, one or more of P8, V9 or 19, 110 or L10, Q38, R39, T40, N41 G42, S43, P44, R45, D82, 183, A84, D85, Y86, Y87, G99, A100, G101, T102, K103, L104, E105, R142, S162, V163, T164, E165, Q166, D167, S 168, and Y173 of the light chain, and/or one or more of Q6, P9, R38, Q39, S40, P41, G42, K43, G44, L45, S84, D86, T87, A88, 189, Y90, Y91, W103, G104, Q105, G106, T107, L108, V109, T110, V111, Y147, E150, P151, V152, T173, F174, P175, A176, V177, Y185, S186, and L187 of the heavy chain (with reference to cetuximab, or analogous residues in other meditope-enabled antibodies) are mutated.

It is contemplated that when the meditope comprising SEQ ID NO: 1 described herein is non-covalently associated to the antibody within the meditope-binding site, position 10 (i.e., Leu10) of the meditope is in close proximity to amino acid 87, according to Kabat numbering, of the antibody (e.g., less than about 3.5 Å). In certain embodiments, the C1 of the meditope is more distant to the amino acid 87 of the antibody than C12 when the meditope comprising SEQ ID NO: 1 described herein is non-covalently associated to the antibody within the meditope-binding site (see, FIG. 5).

In one embodiment, the antibody is cetuximab or a functional fragment of cetuximab comprising the meditope-enabled cavity. Thus, in certain embodiments, the meditope-enabled antibody has a light chain and/or heavy chain variable region with the framework region or regions (FRs) of a meditope-enabled antibody, such as cetuximab, a meditope-enabled trastuzumab, or a meditope-enabled M5A (or FR(s) with at least at or about 75, 80, 85, 90, 91, 92, 93, 94, 95, 96, 97, 98, or 99% identity to the FR(s) of such an antibody).

In some embodiments, the meditope-enabled antibodies are generated by modifying an antibody other than cetuximab (sometimes referred to as the template antibody), such as an antibody having one or more CDRs distinct from those of cetuximab, to confer the ability to bind to one or more of the provided meditopes, such as a meditope comprising any of SEQ ID NOs: 1-24, or variant thereof. The template antibody can be a human or humanized antibody or a mouse antibody. In one aspect, the modifications include substituting residues within the central cavity of the Fab fragment, typically within the framework regions (FRs) of the heavy and light chain variable regions and/or the constant regions to render the template antibody meditope-enabled. For example, where the template antibody is a human or humanized antibody, the modifications generally include substitutions at residues within the heavy and light chain variable region FRs. In some embodiments, such residues are replaced with the corresponding residue present in cetuximab, or comparable amino acid. Thus, in certain embodiments, residues within the FRs of a human or humanized antibody are replaced with corresponding murine residues; in certain embodiments, they are replaced by other residues, such as those having similar functional groups or moieties for interacting with the meditopes. Typically, the residues replaced by corresponding murine (or other) residues are found within the central Fab cavity, and thus are not exposed to the immune system.

In some embodiments, the meditope-enabled antibody comprises an amino acid as defined in Table 1, where specific light chain modifications are shown. Table 2 shows the specific heavy chain modifications. In certain embodiments, the antibody comprises at least one of amino acids as described in Table 1 and/or Table 2. Amino acids in the tables are defined for a specific (Kabat) position to facilitate covalent linking of the meditope peptide with the photoactivatable amino acid upon photoactivation. For example, in some aspects, one or more of P8, V9 or 19, 110 or L10, Q38, R39, T40, N41 G42, S43, P44, R45, D82, 183, A84, D85, Y86, Y87, G99, A100, G101, T102, K103, L104, E105, R142, S162, V163, T164, E165, Q166, D167, S168, and Y173 of the light chain (VL), and/or one or more of Q6, P9, R38, Q39, S40, P41, G42, K43, G44, L45, S84, D86, T87, A88, 189, Y90, Y91, W103, G104, Q105, G106, T107, L108, V109, T110, V111, Y147, E150, P151, V152, T173, F174, P175, A176, V177, Y185, S186, and L187 of the heavy chain (VH) (with reference to cetuximab, or analogous residues in other meditope-enabled antibodies) are mutated.

The antibody can also be modified to increase the efficiency of meditope binding and increase the efficiency of photoconjugation thereof to the antibody. Such modifications include containing one or more of the amino acids identified below in Table 1 and 2. Accordingly, in certain embodiments, the antibody includes one or more of the amino acids listed below in Table 1 and/or Table 2. In Tables 1 and 2, the desired optimized amino acids for efficient photoconjugation are marked bold and italic. In certain embodiments, the antibody comprises at least one of amino acids LC 101 (G), LC 105 (E), HC 44 (G), and/or HC 155 (E).

In certain embodiments, the meditope contains one or more of G42, Q42, N45, G45, K45, G46, K46, A100, K103, R103, E165, and/or S165 of the light chain, and/or one or more of K43, G44 and/or E155 of the heavy chain (with based on Kabat numbering and with reference to cetuximab, or analogous residues in other meditope-enabled antibodies). In certain embodiments, the meditope contains E165 of the light chain (with based on Kabat numbering and with reference to cetuximab, or analogous residues in other meditope-enabled antibodies).

TABLE 1

| Light Chain (LC) | |
|---|---|
| (as defined by Kabat | |
| numbering system) | Amino Acid |
| 39 | R, H, L, Q |
| 42 | G, Q, R, A, M, N |
| 43 | S, A, P, T, G, K |
| 44 | T, A, S, P |
| 45 | N, G, K, R, Q |
| 46 | G, K, L, S, R, V |
| 47 | L, N, S, D, P |
| 48 | L, I, V |
| 84 | A, G, S |
| 87 | Y, I, L, F, A, D |
| 100 | A, G, Q, S |
| 101 | G |
| 102 | T, S |
| 103 | K, R, Q, L, T, E |
| 105 | E, T, V, K |
| 165 | E, S, L, V, W |

TABLE 2

| Heavy Chain (HC) | |
|---|---|
| (as defined by Kabat | |
| numbering system) | Amino Acid |
| 39 | Q, T, W, S |
| 41 | P, R, S, T, A |
| 42 | G, E, V |
| 43 | K, Q, E, N |
| 44 | G, K, R, I, Q, A |
| 91 | A, T, I, S |
| 94 | Y, D |
| 95 | Y, F, L, M, T |
| 108 | Q, G |
| 112 | Q, G, E, A |
| 155 | E, D, G |
| 174 | P, T, V, K, N |

As such, in some embodiments, introducing these amino acid substitutions in a human or humanized antibody do not increase or do not substantially increase the antigenicity of the modified template antibody, in the context of delivery to a human subject. In addition, antigenicity prediction algorithms may be further used to indicate that the human sequence with the point mutations should not be antigenic.

Non-limiting examples of modified antibodies include cetuximab I83E, meditope enabled trastuzumab, meditope enabled panitumumab, meditope enabled ABT-806, meditope enabled gemtuzumab, meditope enabled lintuzumab, meditope enabled clivatuzumab, cetuximab variant 2 (V2) (with LC I83E, K103R, E165D and HC E154S, Q111K), cetuximab variant 3 (V3) (with LC I83E, K103Q, E165P and HC Q11 S, E154D), MBI_Muc1 (meditope enabled HuHMFG1), MBI_CD19_1 (SAR3419), MBI_CD19_2 (MOR208), meditope enabled rituximab, meditope enabled X4-3 anti IDO2 antibody, meditope enabled ocaratuzumab, photo-optimized trastuzumab V2 (Tmab), meditope enabled vadastuximab, photo-optimized panitumumab V2 (Pmab), immunogen EGFR mAb, and photo-optimized anti-CD19 antibody. None-limiting examples of antibody sequences are listed below in Table 3.

TABLE 3

| SEQ ID NO: | Antibody Sequence | Name |
|---|---|---|
| 25 | DILLTQSPVILSVSPGERVSFSCRASQSIGTNIHWYQQRTNGSPRLL | Cetuximab, light |
| | IKYASESISGIPSRFSGSGSGTDFTLSINSVESEDIADYYCQQNNNW | chain |
| | PTTFGAGTKLELKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 26 | QVQLKQSGPGLVQPSQSLSITCTVSGFSLTNYGVHWVRQSPGKGLEW | Cetuximab, heavy |
| | LGVIWSGGNTDYNTPFTSRLSINKDNSKSQVFFKMNSLQSNDTAIYY | chain |
| | CARALTYYDYEFAYWGQGTLVTVSAASTKGPSVFPLAPSSKSTSGGT | |
| | AALGCLVKDYFPEPVTVSWNSGALTSGVHTFPAVLQSSGLYSLSSVV | |
| | TVPSSSLGTQTYICNVNHKPSNTKVDKRVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGN | |
| | VFSCSVMHEALHNHYTQKSLSLSPGK | |
| 27 | DILLTQSPVILSVSPGERVSFSCRASQSIGTNIHWYQQRTNGSPRLL | Cetuximab 183E, |
| | IKYASESISGIPSRFSGSGSGTDFTLSINSVESEDEADYYCQQNNNW | light chain |
| | PTTFGAGTKLELKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 28 | QVQLKQSGPGLVQPSQSLSITCTVSGFSLTNYGVHWVRQSPGKGLEW | Cetuximab |
| | LGVIWSGGNTDYNTPFTSRLSINKDNSKSQVFFKMNSLQSNDTAIYY | 183E, heavy chain |
| | CARALTYYDYEFAYWGQGTLVTVSAASTKGPSVFPLAPSSKSTSGGT | |
| | AALGCLVKDYFPEPVTVSWNSGALTSGVHTFPAVLQSSGLYSLSSVV | |
| | TVPSSSLGTQTYICNVNHKPSNTKVDKRVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGN | |
| | VFSCSVMHEALHNHYTQKSLSLSPGK | |
| 29 | DIQMTQSPSILSASVGDRVTITCRASQDVNTAVAWYQQRTNKAPRLL | Trastuzumab |
| | IYSASFLYSGVPSRFSGSRSGTDFTLTISSLQPEDEADYYCQQHYTT | enabled, light |
| | PPTFGAGTKLEIKRTVAAPSVFIFPPSDEQLKSGTASVVCLLNNFYP | chain |
| | REAKVQWKVDNALQSGNSQESVTEQDSKDSTYSLSSTLTLSKADYEK | |
| | HKVYACEVTHQGLSSPVTKSFNRGEC | |
| 30 | EVQLVESGGGLVQPGGSLRLSCAASGFNIKDTYIHWVRQSPGKGLEW | Trastuzumab |
| | VARIYPTNGYTRYADSVKGRFTISADTSKNTAYLQMNSLRAEDTAIY | enabled, heavy |
| | YCSRWGGDGFYAMDYWGQGTLVTVSSASTKGPSVFPLAPSSKSTSGG | chain |
| | TAALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPG | |
| 31 | DIQMTQSPSILSASVGDRVTITCQASQDISNYLNWYQQRTNKAPRLL | Panitumumab |
| | IYDASNLETGVPSRFSGSGSGTDFTFTISSLQPEDEADYFCQHFDHL | enabled, light |
| | PLAFGAGTKLEIKRTVAAPSVFIFPPSDEQLKSGTASVVCLLNNFYP | chain |
| | REAKVQWKVDNALQSGNSQESVTEQDSKDSTYSLSSTLTLSKADYEK | |
| | HKVYACEVTHQGLSSPVTKSFNRGEC | |
| 32 | QVQLQESGPGLVKPSETLSLTCTVSGGSVSSGDYYWTWIRQSPGKGL | Panitumumab |
| | EWIGHIYYSGNTNYNPSLKSRLTISIDTSKTQFSLKLSSVTAADTAI | enabled, heavy |
| | YYCVRDRVTGAFDIWGQGTMVTVSSASTKGPSVFPLAPCSRSTSEST | chain |
| | AALGCLVKDYFPEPVTVSWNSGALTSGVHTFPAVLQSSGLYSLSSVV | |
| | TVPSSNFGTQTYTCNVDHKPSNTKVDKTVERKCCVECPPCPAPPVAG | |
| | PSVFLFPPKPKDTLMISRTPEVTCVVVDVSHEDPEVQFNWYVDGVEV | |
| | HNAKTKPREEQFNSTFRVVSVLTVVHQDWLNGKEYKCKVSNKGLPAP | |
| | IEKTISKTKGQPREPQVYTLPPSREEMTKNQVSLTCLVKGFYPSDIA | |
| | VEWESNGQPENNYKTTPPMLDSDGSFFLYSKLTVDKSRWQQGNVFSC | |
| | SVMHEALHNHYTQKSLSLSPGK | |
| 33 | DILMTQSPSIMSVSLGDTVSITCHSSQDINSNIGWLQQRTNGSPRGL | ABT-806 enabled, |
| | IYHGTNLDDEVPSRFSGSGSGADYSLTISSLESEDEADYYCVQYAQF | light chain |
| | PWTFGAGTKLEIKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 34 | DVQLQESGPSLVKPSQSLSLTCTVTGYSITSDFAWNWIRQSPGNKLE | ABT-806 enabled, |
| | WMGYISYSGNTRYNPSLKSRISITRDTSKNQFFLQLNSVTIEDTAIY | heavy chain |
| | YCVTAGRGFPYWGQGTLVTVSASTKGPSVFPLAPSSKSTSGGTAALG | |
| | CLVKDYFPEPVTVSWNSGALTSGVHTFPAVLQSSGLYSLSSVYSVPS | |
| | SSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPAP | |
| | IEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |
| 35 | QIVLTQSPAIIVISASPGEKVTITCSASSSISYMHWFQQRTNGSPRL | Gemtuzumab |
| | WIYTTSNLASGVPARFSGSGSGTSYSLTISRMEAEDEADYYCHQRST | enabled, light |
| | YPLTFGAGTKLELKRTVAAPSVFIFPPSDEQLKSGTASVVCLLNNFY | chain |
| | PREAKVQWKVDNALQSGNSQESVTEQDSKDSTYSLSSTLTLSKADYE | |
| | KHKVYACEVTHQGLSSPVTKSFNRGEC | |
| 36 | QVQLQQSGAELAKPGASVKMSCKASGYTFTSYRMHWVKQSPGQGLEW | Gemtuzumab |
| | IGYINPSTGYTEYNQKFKDKATLTADKSSSTAYMQLSSLTFEDSAIY | enabled, heavy |
| | YCARGGGVFDYWGQGTTLTVSSASTKGPSVFPLAPSSKSTSGGTAAL | chain |
| | GCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFS | |
| | CSVMHEALHNHYTQKSLSLSPGK | |
| 37 | DIQMTQSPSILSASVGDRVTITCRASESVDNYGISFMNWFQQRTNKA | Lintuzumab |
| | PRLLIYAASNQGSGVPSRFSGSGSGTDFTLTISSLQPDDEADYYCQQ | enabled, light |
| | SKEVPWTFGAGTKLEIKRTVAAPSVFIFPPSDEQLKSGTASVVCLLN | chain |
| | NFYPREAKVQWKVDNALQSGNSQESVTEQDSKDSTYSLSSTLTLSKA | |
| | DYEKHKVYACEVTHQGLSSPVTKSFNRGEC | |
| 38 | EVQLVQSGAEVKKPGSSVKVSCKASGYTFTDYNMHWVRQSPGQGLEW | Lintuzumab |
| | IGYIYPYNGGTGYNQKFKSKATITADESTNTAYMELSSLRSEDTAIY | enabled, heavy |
| | YCARGRPAMDYWGQGTLVTVSSASTKGPSVFPLAPSSKSTSGGTAAL | chain |
| | GCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFS | |
| | CSVMHEALHNHYTQKSLSLSPGK | |
| 39 | GVHSDIQMTQSPSILSASVGDRVTITCSASQDIGNFLNWYQQRTNKT | Rebmab200 |
| | PRVLIYYTSSLYSGVPSRFSGSGSGTDYTLTISSLQPEDEADYYCQQ | enabled, light |
| | YSKLPLTFGAGTKLELKRTVAAPSVFIFPPSDEQLKSGTASVVCLLN | chain |
| | NFYPREAKVQWKVDNALQSGNSQESVTEQDSKDSTYSLSSTLTLSKA | |
| | DYEKHKVYACEVTHQGLSSPVTKSFNRGEC | |
| 40 | GVHSQVQLVQSGAEVVKPGASVKMSCKASGYTFTGYNIHWVKQSPGQ | Rebmab200 |
| | GLEWIGAIYPGNGDTSYKQKFRGRATLTADTSTSTVYMELSSLRSED | enabled, heavy |
| | SAIYYCARGETARATFAYWGQGTLVTVSSGASTKGPSVFPLAPSSKS | chain |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPP | |
| | CPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYTQKSLSLSPGK | |
| 41 | DIQLTQSPSILSASVGDRVTMTCSASSSVSSSYLYWYQQKTNKAPRL | Clivatuzumab |
| | WIYSTSNLASGVPARFSGSGSGTDFTLTISSLQPEDEADYFCHQWNR | enabled, light |
| | YPYTFGAGTRLEIKRTVAAPSVFIFPPSDEQLKSGTASVVCLLNNFY | chain |
| | PREAKVQWKVDNALQSGNSQESVTEQDSKDSTYSLSSTLTLSKADYE | |
| | KHKVYACEVTHQGLSSPVTKSFNRGEC | |
| 42 | QVQLQQSGAEVKKPGASVKVSCEASGYTFPSYVLHWVKQSPGQGLEW | Clivatuzumab |
| | IGYINPYNDGTQYNEKFKGKATLTRDTSINTAYMELSRLRSDDTAIY | enabled, heavy |
| | YCARGFGGSYGFAYWGQGTLVTVSSASTKGPSVFPLAPSSKSTSGGT | chain |
| | AALGCLVKDYFPEPVTVSWNSGALTSGVHTFPAVLQSSGLYSLSSVV | |
| | TVPSSSLGTQTYICNVNHKPSNTKVDKRVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGN | |
| | VFSCSVMHEALHNHYTQKSLSLSPGK | |
| 43 | DILLTQSPVILSVSPGERVSFSCRASQSIGTNIHWYQQRTNGSPRLL | MBI2_V2; |
| | IKYASESISGIPSRFSGSGSGTDFTLSINSVESEDEADYYCQQNNNW | Cetuximab with |
| | PTTFGAGTRLELKRTVAAPSVFIFPPSDEQLKSGTASVVCLLNNFYP | LC I83E, K103R, |
| | REAKVQWKVDNALQSGNSQESVTDQDSKDSTYSLSSTLTLSKADYEK | E165D and HC |
| | HKVYACEVTHQGLSSPVTKSFNRGEC | E154S, Q111K, |
| | | light chain |
| 44 | QVQLKQSGPGLVQPSQSLSITCTVSGFSLTNYGVHWVRQSPGKGLEW | MBI2_V2, heavy |
| | LGVIWSGGNTDYNTPFTSRLSINKDNSKSQVFFKMNSLQSNDTAIYY | chain |
| | CARALTYYDYEFAYWGKGTLVTVSAASTKGPSVFPLAPSSKSTSGGT | |
| | AALGCLVKDYFPSPVTVSWNSGALTSGVHTFPAVLQSSGLYSLSSVV | |
| | TVPSSSLGTQTYICNVNHKPSNTKVDKRVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGN | |
| | VFSCSVMHEALHNHYTQKSLSLSPGK | |
| 45 | DILLTQSPVILSVSPGERVSFSCRASQSIGTNIHWYQQRTNGSPRLL | MBI2_V3; |
| | IKYASESISGIPSRFSGSGSGTDFTLSINSVESEDEADYYCQQNNNW | Cetuximab with |
| | PTTFGAGTQLELKRTVAAPSVFIFPPSDEQLKSGTASVVCLLNNFYP | LC I83E, K103Q, |
| | REAKVQWKVDNALQSGNSQESVTPQDSKDSTYSLSSTLTLSKADYEK | E165PHC Q111S, |
| | HKVYACEVTHQGLSSPVTKSFNRGEC | E154D, light chain |
| 46 | QVQLKQSGPGLVQPSQSLSITCTVSGFSLTNYGVHWVRQSPGKGLEW | MBI2_V3, heavy |
| | LGVIWSGGNTDYNTPFTSRLSINKDNSKSQVFFKMNSLQSNDTAIYY | chain |
| | CARALTYYDYEFAYWGSGTLVTVSAASTKGPSVFPLAPSSKSTSGGT | |
| | AALGCLVKDYFPDPVTVSWNSGALTSGVHTFPAVLQSSGLYSLSSVV | |
| | TVPSSSLGTQTYICNVNHKPSNTKVDKRVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGN | |
| | VFSCSVMHEALHNHYTQKSLSLSPGK | |
| 47 | DIQMTQSPSILSASVGDRVTITCKSSQSLLYSSNQKIYLAWYQQKTN | MBI_Muc1; |
| | GAPKLLIYIWASTRESGVPSRFSGSGSGTDFTFTISSLQPEDEADYY | enabled |
| | CQQYYRYPRTFGQGTKVEIKRTVAAPSVFIFPPSDEQLKSGTASVVC | HuHMFG1, light |
| | LLNNFYPREAKVQWKVDNALQSGNSQESVTEQDSKDSTYSLSSTLTL | chain |
| | SKADYEKHKVYACEVTHQGLSSPVTKSFNRGEC | |
| 48 | QVQLVQSGAEVKKPGASVKVSCKASGYTFSAYWIEWVRQSPGKGLEW | MBI_Muc1, |
| | VGEILPGSGYTRYNEKFKGRVTVTRDTSTNTAYMELSSLRANDTAIY | enabled |
| | YCARSYDFAWFAYWGQGTLVTVSSASTKGPSVFPLAPSSKSTSGGTA | HuHMF G1, heavy |
| | ALGCLVKDYFPEPVTVSWNSGALTSGVHTFPAVLQSSGLYSLSSVVT | chain |
| | VPSSSLGTQTYICNVNHKPSNTKVDKKVEPKSCDKTHTCPPCPAPEL | |
| | LGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPS | |
| | DIAVEWESNGQPENNYKTTPPVLDSDGSFFLYSKLTVDKSRWQQGNV | |
| | FSCSVMHEALHNHYTQKSLSLSPGK | |
| 49 | EIVLTQSPAIMSASPGERVTMTCSASSGVNYMHWYQQKTNGSPRRWI | MBI_CD191; |
| | YDTSKLASGVPARFSGSGSGTDYSLTISSMEPEDEADYYCHQRGSYT | (SAR3419), light |
| | FGGGTKLEIKRTVAAPSVFIFPPSDEQLKSGTASVVCLLNNFYPREA | chain |
| | KVQWKVDNALQSGNSQESVTEQDSKDSTYSLSSTLTLSKADYEKHKV | |
| | YACEVTHQGLSSPVTKSFNRGEC | |
| 50 | QVQLVQPGAEVVKPGASVKLSCKTSGYTFTSNWMHWVKQSPGQGLEW | MBI_CD19_1, |
| | IGEIDPSDSYTNYNQNFQGKAKLTVDKSTSTAYMEVSSLRSDDTAIY | (SAR3419), heavy |
| | YCARGSNPYYYAMDYWGQGTSVTVSSASTKGPSVFPLAPSSKSTSGG | chain |
| | TAALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| 51 | DIVMTQSPAILSLSPGERATLSCRSSKSLQNVNGNTYLYWFQQKTNG | MBI_CD192; |
| | SPRLLIYRMSNLNSGVPDRFSGSGSGTEFTLTISSLEPEDEADYYCI | (MOR208), light |
| | QHLEYPITFGAGTKLEIKRTVAAPSVFIFPPSDEQLKSGTASVVCLL | chain |
| | NNFYPREAKVQWKVDNALQSGNSQESVTEQDSKDSTYSLSSTLTLSK | |
| | ADYEKHKVYACEVTHQGLSSPVTKSFNRGEC | |
| 52 | EVQLVESGGGLVKPGGSLKLSCAASGYTFTSYVMHWVRQSPGKGLEW | MBI_CD192, |
| | IGYINPYNDGTKYNEKFQGRVTISSDKSISTAYMELSSLRSEDTAIY | (MOR208), heavy |
| | YCARGTYYYGTRVFDYWGQGTLVTVSSASTKGPSVFPLAPSSKSTSG | chain |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPDVFLFPPKPKDTLMISRTPEVTCVVVDVSHEDPEVQFNWY | |
| | VDGVEVHNAKTKPREEQFNSTFRVVSVLTVVHQDWLNGKEYKCKVSN | |
| | KALPAPEEKTISKTKGQPREPQVYTLPPSREEMTKNQVSLTCLVKGF | |
| | YPSDIAVEWESNGQPENNYKTTPPMLDSDGSFFLYSKLTVDKSRWQQ | |
| | GNVFSCSVMHEALHNHYTQKSLSLSPGK | |
| 53 | QIVLSQSPVILSASPGEKVTMTCRASSSVSYIHWFQQRTNGSPRPWI | Rituximab |
| | YATSNLASGVPVRFSGSGSGTSYSLTISRVEAEDEADYYCQQWTSNP | enabled, light |
| | PTFGAGTKLEIKRTVAAPSVFIFPPSDEQLKSGTASVVCLLNNFYPR | chain |
| | EAKVQWKVDNALQSGNSQESVTEQDSKDSTYSLSSTLTLSKADYEKH | |
| | KVYACEVTHQGLSSPVTKSFNRGEC | |
| 54 | QVQLQQPGAELVKPGASVKMSCKASGYTFTSYNMHWVKQSPGRGLEW | Rituximab |
| | IGAIYPGNGDTSYNQKFKGKATLTADKSSSTAYMQLSSLTSEDSAIY | enabled, heavy |
| | YCARSTYYGGDWYFNVWGAGTTVTVSAASTKGPSVFPLAPSSKSTSG | chain |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSN | |
| | KALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPGK | |
| 55 | DIQMTQSPVILSASVGETVTITCRASENIHNYLAWYQQRTNGSPRLL | X4-3 anti IDO2 |
| | VYNPKNLADGVPSRFSGSGSGTQYSLNINSLQPEDEGDYYCQHFWNT | enabled, light |
| | PPTFGAGTRLEIKRTVAAPSVFIFPPSDEQLKSGTASVVCLLNNFYP | chain |
| | REAKVQWKVDNALQSGNSQESVTEQDSKDSTYSLSSTLTLSKADYEK | |
| | HKVYACEVTHQGLSSPVTKSFNRGEC | |
| 56 | EVQLQQSGPELVKPGASVQISCKTSGYTFTEYTMHWVKQSHGKSLEW | X4-3 anti IDO2 |
| | LGIIHPDNGITRYNQKFKAKATLTEDKSSRTAYMELRSLTSEDSAIY | enabled, heavy |
| | YCARRYYGNFDYALDYWGQGTSVTVSSASTKGPSVFPLAPSSKSTSG | chain |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSN | |
| | KALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPGK | |
| 57 | EIVLTQSPVILSLSPGERATLSCRASSSVPYIHWYQQRTNGSPRLLI | Ocaratuzumab |
| | YATSALASGIPDRFSGSGSGTDFTLTISRLEPEDEADYYCQQWLSNP | enabled, light |
| | PTFGAATKLEIKRTVAAPSVFIFPPSDEQLKSGTASVVCLLNNFYPR | chain |
| | EAKVQWKVDNALQSGNSQESVTEQDSKDSTYSLSSTLTLSKADYEKH | |
| | KVYACEVTHQGLSSPVTKSFNRGEC | |
| 58 | EVQLVQSGAEVKKPGESLKISCKGSGRTFTSYNMHWVRQSPGKGLEW | Ocaratuzumab |
| | MGAIYPLTGDTSYNQKSKLQVTISADKSISTAYLQWSSLKASDTAIY | enabled, heavy |
| | YCARSTYVGGDWQFDVWGKGTTVTVSSASTKGPSVFPLAPSSKSTSG | chain |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPSVFLFPPKIKDTLMISRTPEVTCVVVDVSHEDPEVKFNWY | |
| | VDGVEVHNAKTKPREEQYNSTYRVVSVLTVLHQDWLNGKEYKCKVSN | |
| | KALPAPIEKTISKQKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPG | |
| 59 | DIQMTQSPVILSASVGDRVTITCRASQDVNTAVAWYQQRTNGSPKLL | Photo optimized |
| | IYSASFLYSGVPSRFSGSRSGTDFTLTISSLQPEDEADYYCQQHYTT | trastuzumab; |
| | PPTFGQGTKVEIKRTVAAPSVFIFPPSDEQLKSGTASVVCLLNNFYP | meditope enabled |
| | REAKVQWKVDNALQSGNSQESVTEQDSKDSTYSLSSTLTLSKADYEK | (Tmab), light |
| | HKVYACEVTHQGLSSPVTKSFNRGEC | chain |
| 60 | EVQLVESGGGLVQPGGSLRLSCAASGFNIKDTYIHWVRQSPGKGLEW | Photo optimized |
| | VARIYPTNGYTRYADSVKGRFTISADTSKNTAYLQMNSLRAEDTAIY | trastuzumab; |
| | YCSRWGGDGFYAMDYWGQGTLVTVSSASTKGPSVFPLAPSSKSTSGG | meditope enabled |
| | TAALGCLVKDYFPEPVTVSWNSGALTSGVHTFPAVLQSSGLYSLSSV | (Tmab), heavy |
| | VTVPSSSLGTQTYICNVNHKPSNTKVDKKVEPKSCDKTHTCPPCPAP | chain |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPG | |
| 61 | DILMTQSPVILSASVGDRVTINCKASQDINSYLSWYQQRTNGSPKTL | Photo optimized |
| | IYRANRLVDGVPSRFSGSGSGQDYTLTISSLQPEDEADYYCLQYDEF | Vadastuximab; |
| | PLTFGGGTKVEIKRTVAAPSVFIFPPSDEQLKSGTASVVCLLNNFYP | meditope enabled, |
| | REAKVQWKVDNALQSGNSQESVTEQDSKDSTYSLSSTLTLSKADYEK | light chain |
| | HKVYACEVTHQGLSSPVTKSFNRGEC | |
| 62 | EDAMDYWGQGTTVTVSSASTKGPSVFPLAPSSKSTSGGTAALGCLVK | Photo optimized |
| | DYFPEPVTVSWNSGALTSGVHTFPAVLQSSGLYSLSSVVTVPSSSLG | Vadastuximab; |
| | TQTYICNVNHKPSNTKVDKKVEPKSCDKTHTCPPCPAPELLGGPCVF | meditope enabled, |
| | LFPPKPKDTLMISRTPEVTCVVVDVSHEDPEVKFNWYVDGVEVHNAK | heavy chain |
| | TKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMH | |
| | EALHNHYTQKSLSLSPGKIYPGDGSTKYNEKFKAKATLTADTSTSTA | |
| | YMELRSLRSDDTAVYYCASGYEDAMDYWGQGTTVTVSSA | |
| 63 | DILMTQSPVILSASVGDRVTITCQASQDISNYLNWYQQRTNGSPKLL | Photo optimized |
| | IYDASNLETGVPSRFSGSGSGTDFTFTISSLQPEDIADYYCQHFDHL | panitumumab; |
| | PLAFGGGTKVEIKRTVAAPSVFIFPPSDEQLKSGTASVVCLLNNFYP | meditope enabled |
| | REAKVQWKVDNALQSGNSQESVTEQDSKDSTYSLSSTLTLSKADYEK | (Pmab), light |
| | HKVYACEVTHQGLSSPVTKSFNRGEC | chain |
| 64 | QVQLQESGPGLVKPSETLSLTCTVSGGSVSSGDYYWTWIRQSPGKGL | Photo optimized |
| | EWIGHIYYSGNTNYNPSLKSRLTISIDTSKTQFSLKLSSVTAADTAI | panitumumab; |
| | YYCVRDRVTGAFDIWGQGTMVTVSSASTKGPSVFPLAPCSRSTSEST | meditope enabled |
| | AALGCLVKDYFPEPVTVSWNSGALTSGVHTFPAVLQSSGLYSLSSVV | (Pmab), heavy |
| | TVPSSNFGTQTYTCNVDHKPSNTKVDKTVERKCCVECPPCPAPPVAG | chain |
| | PSVFLFPPKPKDTLMISRTPEVTCVVVDVSHEDPEVQFNWYVDGVEV | |
| | HNAKTKPREEQFNSTFRVVSVLTVVHQDWLNGKEYKCKVSNKGLPAP | |
| | IEKTISKTKGQPREPQVYTLPPSRDELTKNQVSLTCLVKGFYPSDIA | |
| | VEWESNGQPENNYKTTPPMLDSDGSFFLYSKLTVDKSRWQQGNVFSC | |
| | SVMHEALHNHYTQKSLSLSPGK | |
| 65 | EIVLTQSPVILSASPGERVTITCSASSGVNYMHWYQQRTNGSPKRWI | Photo-optimized |
| | YDTSKLASGVPARFSGSGSGTDYSLTISSMEPEDEADYYCHQRGSYT | immunogen EGFR |
| | FGGGTKLEIKRTVAAPSVFIFPPSDEQLKSGTASVVCLLNNFYPREA | antibody; meditope |
| | KVQWKVDNALQSGNSQESVTEQDSKDSTYSLSSTLTLSKADYEKHKV | enabled, light |
| | YACEVTHQGLSSPVTKSFNRGEC | chain |
| 66 | QVQLVQPGAEVVKPGASVKLSCKTSGYTFTSNWMHWVKQSPGQGLEW | Photo-optimized |
| | IGEIDPSDSYTNYNQNFQGKAKLTVDKSTSTAYMEVSSLRSDDTAIY | immunogen EGFR |
| | YCARGSNPYYYAMDYWGQGTSVTVSSASTKGPSVFPLAPSSKSTSGG | antibody; meditope |
| | TAALGCLVKDYFPEPVTVSWNSGALTSGVHTFPAVLQSSGLYSLSSV | enabled, heavy |
| | VTVPSSSLGTQTYICNVNHKPSNTKVDKKVEPKSCDKTHTCPPCPAP | chain |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| 67 | DIQMTQSPVILSASVGDRVTITCKASQDINNYLAWYQQRTNGSPKLL | Photo-optimized |
| | IHYTSTLHPGIPSRFSGSGSGRDYSFSISSLEPEDEADYYCLQYDNL | CD19 antibody; |
| | LYTFGQGTKLEIKRTVAAPSVFIFPPSDEQLKSGTASVVSLLNNFYP | meditope enabled, |
| | REAKVQWKVDNALQSGNSQESVTEQDSKDSTYSLSSTLTLSKADYEK | light chain |
| | HKVYACEVTHQGLSSPVTKSFNRGEC | |
| 68 | QVQLVQSGAEVAKPGASVKLSCKASGYTFTSYWMQWVKQRPGQGLEC | Photo-optimized |
| | IGTIYPGDGDTTYTQKFQGKATLTADKSSSTAYMQLSSLRSEDSAIY | CD19 antibody; |
| | YCARYDAPGYAMDYWGQGTLVTVSSASTKGPSVFPLAPSSKSTSGGT | meditope enabled, |
| | AALGCLVKDYFPEPVTVSWNSGALTSGVHTFPAVLQSSGLYSLSSVV | heavy chain |
| | TVPSSSLGTQTYICNVNHKPSNTKVDKKVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSWRQQGN | |
| | VFSCSVMHEALHNHYTQKSLSLSPG | |

### 4. Active Agents

The antibody-meditope conjugate as described herein may be bound, e.g., via a covalent bond, to an active agent. The active agent can be a therapeutic agent, a diagnostic agent, or a detectable agent, and is also referred to as a toxin or payload. Exemplary active agents include, but are limited to, 5-azacitidine, 5-fluorouracil, 6-mercaptopurine, 6-thioguanine, adriamycin, aldesleukin, alitretinoin, all-transretinoic acid, alrubicin, altretamine, amethopterin, amifostine, aminocamptothecin, aminoglutethimide, amsacrine, anagrelide, anastrozole, arabinosylcytosine, asparaginase, azacytidine, bendamustine, bexarotene, bleomycin, bortezomib, busulfan, calcium, leucovorin, Calicheamicin, canertinib, carboplatin, carmustine, chlorambucil, cisplatin, citrovorum factor, cladribine, cyclophosphamide, cytarabine, dactinomycin, darbepoetin alfa, dasatinib, daunomycin, daunorubicin, decitabine, dexamethasone, dexrazoxane, docetaxel, doxifluridine, doxorubicin, eniluracil, epirubicin, epoetin alfa, erlotinib, estramustine, etanercept, etoposide, exemestane, filgrastim, floxuridine, fludarabine, fluorouracil, fluoxymesterone, flutamide, fulvestrant, gefitinib, gemcitabine, goserelin, hexamethylmelamine, hydrocortisone, ifosfamide, imatinib mesylate, interferon alpha, interleukin-2, interleukin-11, irinotecan, isotretinoin, ixabepilone, lapatinib, lenalidomide, letrozole, leuprolide, liposomal Ara-C, lomustine, mechlorethamine, megestrol, melphalan, mesna, methotrexate, methylprednisolone, mitomycin C, mitotane, nelarabine, nilutamide, octreotide, oprelvekin, oxaliplatin, paclitaxel, pamidronate, PEG Interferon, PEG-L-asparaginase, pegaspargase, pegfilgrastim, plicamycin, prednisolone, prednisone, procarbazine, pyrrolobenzodiazepine, ralitrexed, raloxifene, sapacitabine, sargramostim, satraplatin, semustine, sorafenib, sunitinib, tamoxifen, tegafur, tegafur-uracil, temozolamide, temsirolimus, teniposide, thioguanine, thiotepa, topotecan, toremifene, vinblastine, vincristine, vindestine, vinorelbine, vorinostat, actinomycin-D, Amanitin and derivatives thereof, arsenic trioxide, bacillus calmette-guerin (BCG), bicalutamide, capecitabine, chlorodeoxyadenosine, colchicine, cortisone, decarbazine, denileukin diftitox, dolastatin, duocarmycin, duostatin (auristatin derivative), emtansine, everolimus, flavopiridol, gimatecan, hyalauronic acid, hydroxyurea, idarubicin, interferon gamma, leucovorin, macrophage-colony stimulating factor, mercaptopurine, mitoxantrone, monomethyl auristatins (MMA) such as MMAD, MMAE and MMAF, ozogamicin, pemetrexed, pentostatin, ravtansine, romiplostim, streptozocin, thalidomide, transferrin, trimitrexate, tubulysins, and zoledronic acid.

The diagnostic and therapeutic agents include any such agent, which are well-known in the relevant art. Among the imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, galactosidase, glucoronidase or lactamase. Such enzymes may be used in combination with a chromogen, a fluorogenic compound or a luminogenic compound to generate a detectable signal.

Radioactive substances that may be used as imaging or therapeutic agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Th, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal crosslinking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

Exemplary therapeutic agents include, but are not limited to, drugs, chemotherapeutic agents, therapeutic antibodies and antibody fragments, toxins, radioisotopes, enzymes (e.g., enzymes to cleave prodrugs to a cytotoxic agent at the site of the tumor), nucleases, hormones, immunomodulators, antisense oligonucleotides, RNAi molecules (e.g., siRNA or shRNA), chelators, boron compounds, photoactive agents and dyes. The therapeutic agent may also include a metal, metal alloy, intermetallic or core-shell nanoparticle bound to a chelator that acts as a radiosensitizer to render the targeted cells more sensitive to radiation therapy as compared to healthy cells. Further, the therapeutic agent may include paramagnetic nanoparticles for MRI contrast agents (e.g., magnetite or Fe₃O₄) and may be used with other types of therapies (e.g., photodynamic and hyperthermal therapies and imaging (e.g., fluorescent imaging (Au and CdSe)).

Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as therapeutic agents in accordance with the embodiments of the disclosure include, but are not limited to, 13-cis-retinoic acid, 2-chlorodeoxyadenosine, 5-azacitidine, 5-fluorouracil, 6-mercaptopurine, 6-thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, auristatin, dimethylvaline-valine-dolaisoleuinedolaproine-phenylalanine-p-phenylenediamine (AFP), MonoMethyl Dolastatin 10 (MMAD), dovaline-valine-dolaisoleuinedolaproine-phenylalanine (MMAF), monomethyl auristatin E (MMAE), auromycins, azacytidine, bacillus calmette-guerin (BCG), bendamustine, bevacizumab, etanercept, bexarotene, bicalutamide, bismuth, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, cc1065, darbepoetin alfa, dasatinib, daunomycin, decitabine, denileukin diftitox, dexamethasone, dexasone, dexrazoxane, dactinomycin, daunorubicin, decarbazine, docetaxel, dolostatins, doxorubicin, doxifluridine, eniluracil, epirubicin, epoetin alfa, erlotinib, everolimus, exemestane, estramustine, ethidium bromide, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte—colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, maytonsinoids, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, ricin, ricin A-chain, rituximab, romiplostim, ralitrexed, sapacitabine, sargramostim, satraplatin, sorafenib, sunitinib, semustine, streptozocin, taxol, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, yttrieum, or zoledronic acid.

Therapeutic antibodies and functional fragments thereof, that may be used as therapeutic agents in accordance with the embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab and other antibodies associated with specific diseases listed herein.

Toxins that may be used as therapeutic agents in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as therapeutic agents in accordance with the embodiments of the disclosure include, but are not limited to, ³²P, ⁸⁹Sr, ⁹⁰Y, ^{99m}Tc, ⁹⁹Mo, ¹³¹I, ¹⁵³Sm, ¹⁷⁷Lu, ¹⁸⁶Re, ²¹³Bi, ²²³Ra and ²²⁵Ac.

Active agents can be conjugated by any means known in the art. The active agent may be covalently bound to either the N-terminus or the C-terminus, optionally via a linker. In some embodiments, the agent is conjugated to a lysine, tyrosine, glutamic acid, aspartic acid, formylglycine or aldehyde tag, an unnatural amino acid, etc. A formylglycine or aldehyde tag can comprise a 6-amino acid (LCTPSR; SEQ ID NO: 69) or 13-amino acid (LCTPSRGSLFTGR, SEQ ID NO: 70) sequence that can be recognized and targeted by formylglycine-generating enzyme (FGE) to generate a reactive aldehyde group. The reactive aldehyde group can be useful in subsequence reactions comprising conjugating meditopes to agents. See Carrico et al., Nature Chemical Biology 3, 321-322 (2007).

In certain embodiments, the agent is conjugated to the meditope via the side chain of a lysine or a cysteine residue, the N-terminus, or another suitable functional group present on the meditope. The main chemistry used for amine or thiol (e.g., lysine or cysteine) conjugation involves formation of a covalent bond using activated esters of the active agent to be conjugated. Examples of such reagents include O-succinimide reagents, such as N-hydroxysuccinimidyl (NHS), including hydroxybenzotriazole and fluoro- or nitro-phenyl derivatives thereof, or sulfo-NHS esters, maleimido derivatives, haloacetyl derivatives, such as iodoacetamido derivatives, disulfide conjugates, such as linkers containing pyridyldisulfide moieties, isothiocyanate derivatives, Traut's reagent, click chemistry, and the like. The conjugation can be done in one step where the activated ester is present on the active agent to be conjugated, and thus a covalent bond is formed by contacting the meditope with the active agent. A two-step conjugation may also be employed. This type of conjugation proceeds with modification of a lysine residue of the meditope to introduce chemical functionalities that are able to subsequently react with specific reactive groups present on the drug. Exemplary methods include, but are not limited to, the use of O-succinimide reagents for the introduction of maleimido, iodoacetamido, or pyridyldisulfide moieties on the lysine residue of the meditope. See, e.g., Brun, et al. Antibody-Drug Conjugates, Methods in Molecular Biology, Laurent Ducry (ed.), vol. 1045, pp. 173-187. The linkers may also further comprise spacers, such as an alkylene or polyethylene glycol chain.

### 5. Methods

Provided herein are antibody-meditope conjugates, and compositions comprising the same, for delivering agents to a site or cell (e.g., imaging, therapeutic and/or diagnostic agents). A large variety of diagnostic and therapeutic moieties and combinations thereof may be utilized, thereby, providing for highly stable and/or versatile drug delivery and/or diagnostic compositions. Thus, the antibody-meditope conjugates described herein have a broad impact on the mAb delivery field and they provide useful methods for the treatment and diagnosis of various diseases and conditions, including cancers, such as those against which use of an antibody is indicated. For example, antibody-meditope conjugates directed against EGFR-positive cancers, including colorectal and squamous cell carcinoma head and neck cancers, would benefit from use of where cetuximab and a meditope. Additionally, modifying other therapeutic antibodies to generate meditope-enabled versions of such antibodies allows for the antibody-meditope conjugates disclosed herein to be utilized in methods for the treatment and diagnosis of several other cancers, diseases and other conditions.

Accordingly, provided are methods and uses of the antibody-meditope conjugates, and compositions comprising the same, including therapeutic and diagnostic uses. Also provided are pharmaceutical compositions containing the meditopes (including variant and multivalent meditopes and meditope fusion proteins) for use in such diagnostic and therapeutic methods.

In some embodiments, the provided antibody-meditope conjugates, and compositions comprising the same, are used in treatment, diagnosis or imaging of a disease or condition, including any cancer, disease or other condition that may be treated or targeted using a therapeutic antibody. Cancers avoid immune surveillance by actively suppressing the immune system. One method envisioned for counteracting this immunosuppression is through vaccination using epitopes of antigens that are either uniquely expressed or over-expressed by the tumor cells. For example, monoclonal antibodies (mAbs) that block signaling pathways, sequester growth factor and/or induce an immune response have been successfully implemented in the clinic to treat cancer and other diseases.

Accordingly, in certain embodiments, the present disclosure provides compositions and methods for treating cancer. In one embodiment, the present disclosure provides a method for treating cancer in a patient in need thereof, comprising administering to the patient an effective amount of an antibody-meditope conjugate comprising a cyclic meditope as described above and an antibody comprising a meditope-enabled cavity, wherein the meditope further comprises a therapeutic agent and is covalently bonded to the antibody. This antibody-meditope conjugate serves to target the therapeutic agent, such as a chemotherapeutic drug, to a tumor site. In certain embodiments, the cancer is a solid tumor.

Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

### 6 Pharmaceutical Compositions, Dosing and Administration

Provided herein are pharmaceutical compositions comprising the antibody-meditope conjugates and one or more pharmaceutically acceptable excipient or carrier. “Pharmaceutically acceptable excipients” and “pharmaceutically acceptable carriers” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like., that do not deleteriously react with the meditopes and conjugates of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In certain embodiments, the composition described herein is administered intravenously.

The conjugates or a composition described herein can be administered alone or can be co-administered to the patient, at the same time, just prior to, or just after administration of one or more additional therapies. Co-administration is meant to include simultaneous or sequential administration of the conjugates individually or in combination (more than one conjugates or agent). Thus, the preparations can also be combined, when desired, with other active substances (e.g. to reduce metabolic degradation).

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed 7:623-645, 1995; as biodegradable and injectable gel formulations (see, e.g., Gao Phann. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989). The compositions can also be delivered as nanoparticles.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds disclosed herein. Adjustment and manipulation of established dosages (e.g., frequency and duration) are well within the ability of those skilled in the art. In certain embodiments, the compositions are delivered at an amount of from about 0.5 mg to about 1000 mg in 24 hours, or about 1 mg, about 5 mg, about 10 mg, about 15 mg, about 25 mg, about 50 mg about 75 mg, about 100 mg, about 150 mg, about 200 mg, about 250 mg, about 300 mg, about 400 mg, about 500 mg, about 600 mg, about 700 mg, about 800 mg, about 900 mg, or about 1000 mg in 24 hours.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

### 7. Synthesis

Provided herein are methods for preparing an antibody-meditope conjugate, comprising contacting a cyclic peptide (i.e., meditope) which comprises a photoreactive functional group capable of forming a covalent bond upon irradiation with UV light as described herein, with an antibody comprising a meditope-enabled cavity, under conditions to allow formation of a non-covalent interaction between the meditope and one or more amino acids in the meditope-enabled cavity, and irradiating the meditope with UV light to generate a covalent bond between a side chain of an amino acid of the meditope and the antibody.

As used herein, the term “photo peptide(s),” “photo enabled peptide(s),” “photo meditope(s)” or “photo enabled meditope(s)” refer to peptides or meditopes that comprises at least one photoreactive functional group.

It is believed that the covalent bond formation is enhanced by the tight and selective noncovalent association of the meditope to the antibody within the meditope-enabled cavity. It is further contemplated that when the disulfide bridge between C1 and C12 of the meditope is closer to the entrance of the meditope-enabled cavity than the majority of amino acids 2-11 of the meditope, the photoreactive functional group capable of forming a covalent bond is situated in a favorable position to allow efficient formation covalent bond formation to the antibody (see FIG. 5).

In embodiments, the meditope binds to the meditope-enabled cavity (e.g., functional groups within the antibody backbone or of the side chains of amino acids of the antibody) with a K_{D} of less than about 10 μM, or less than about 9 μM, or less than about 8 μM, or less than about 7 μM, or less than about 6 μM, or less than about 5 μM, or less than about 4 μM, or less than about 3 μM, or less than about 2 μM, or less than about 1 μM, or less than about 0.5 μM, or less than about 100 nM, or less than about 90 nM, or less than about 80 nM, or less than about 70 nM, or less than about 60 nM, or less than about 50 nM, or less than about 10 nM, or less than about 1 nM.

The non-covalent meditope antibody complex formation is facilitated by incubating the meditope with meditope-enabled antibody for at least about 20 minutes, at least about 30 minutes, or about 30 minutes at room temperature, or a decreased temperature, such as about 4° C. The concentration of the antibody is typically about 5-10 mg/mL, or about 6 mg/mL. The concentration of the meditope is typically about 100-120 mg/mL, or about 2.5 to 3 times greater than the antibody, as defined by the equilibrium constant.

The wavelength used for irradiating the non-covalent meditope antibody complex can be tailored to the specific requirements of the photoreactive functional group used. In certain embodiments, the wavelength used for irradiating the non-covalent meditope antibody complex is long wave ultraviolet of from about 315 nm to about 400 nm, or about 350 nm, or about 365 nm. An advantage of the conjugation method described herein is that the wavelength used for covalent bond formation does not have a significantly deleterious effect of any component of the conjugate, including the antibody or the therapeutic agent.

Following a single irradiation event, efficiency of covalent binding can be assessed using standard methods. Any unbound peptide can be removed using chromatography, size filtration, etc. After the irradiation with UV light, the efficiency of the covalent bond formation between the meditope and the antibody is typically at least about 15%, or at least about 20%, or at least about 25%, or at least about 30%, or greater than about 30%.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Any intermediates can be purified or used in the following steps without isolation and/or purification.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The meditope can be synthesized using standard peptide synthetic methods known in the art. For example, doubly protected cysteine (e.g., Fmoc-Cys(TrT)-OH) can be first coupled to a resin and the peptide chain assembled using standard peptide coupling chemistry, such as the use of phosphonium salts, tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts, and the like. After assembly of the linear meditope, the thio-protecting groups can be removed and the meditope can be cyclized to form the disulfide bond (or bridge). This step can be prior to, or after, the peptide is cleaved from the resin.

The unnatural amino acids having a photoreactive functional group on the side chain can be synthesized according to known methods (see, e.g., Kauer, J. C., et al. J Biol Chem, 1986, 261(23), 10695-10700; Yang, T., Chem Sci, 2015, 6, 1011-1017; and US 20090211893 A1). It is contemplated that amino acids having either configuration (D or L) may be incorporated.

The diazirine-containing amino acids shown below are referred to herein as (i) “photo-Met”, “L-Photo-methionine” or “L-2-amino-5,5′-azi-hexanoic acid” and (ii) “photo-Leu” or “L-Photo-leucine” or “L-2-amino-4,4-azi-pentanoic acid”. The nomenclature “photo-Xaa” has been adopted as it is indicative of that naturally occurring amino acid, and in some embodiments, which the amino acid it is intended to substitute. Photo-Met and photo-Leu are available from commercial sources.

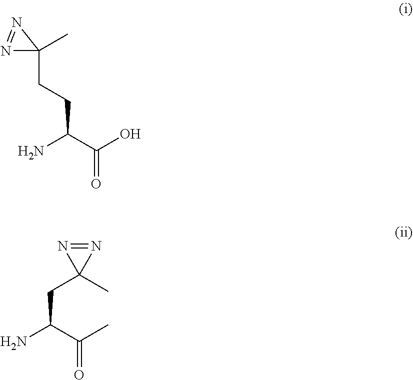

It is contemplated that, in certain embodiments and with certain meditope-enabled antibodies, the covalent bond forming reaction can be facilitated by lengthening the side chain of the amino acid comprising the photoreactive functional group. Accordingly, the diazirine-containing amino acids shown below (i.e., (S)-2-amino-5-(3-methyl-3H-diazirin-3-yl)pentanoic acid and (S)-2-amino-6-(3-methyl-3H-diazirin-3-yl)hexanoic acid can be used in the meditopes described herein.

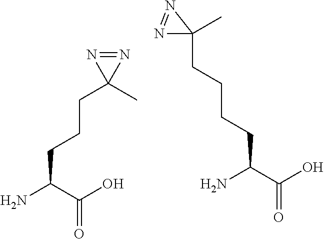

The diazirine-containing amino acids shown above can be synthesized according to methods known in the art using the appropriate starting materials (see, e.g., Yang, T., Chem Sci, 2015, 6, 1011-1017; and US 20090211893 A1). Exemplary methods are also shown below in Scheme 1, where PG is an amine protecting group (e.g., BOC, Fmoc, etc.), R¹⁰⁰ is alkyl, and z is 1 to 11.

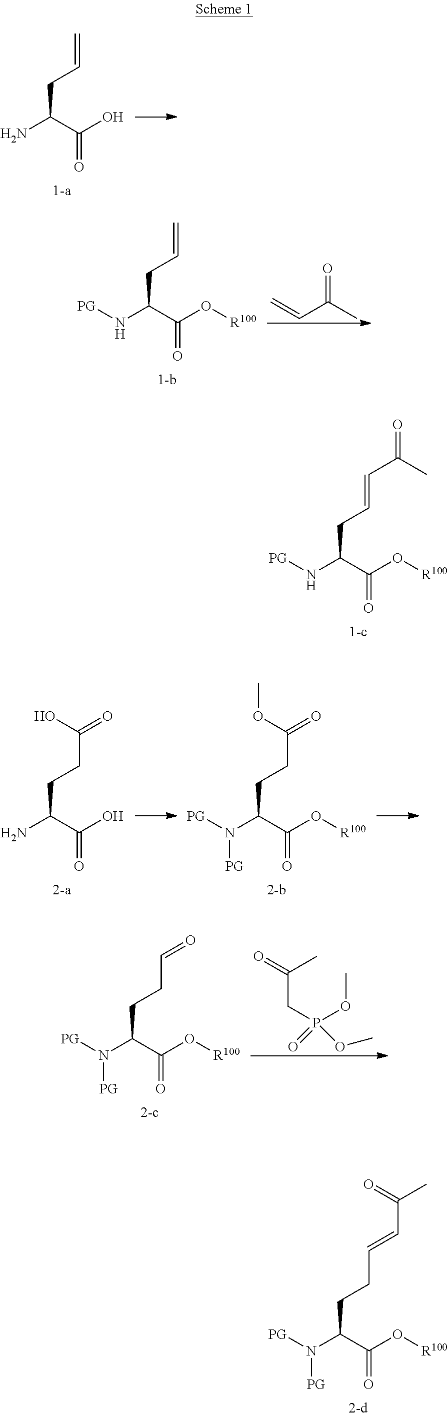

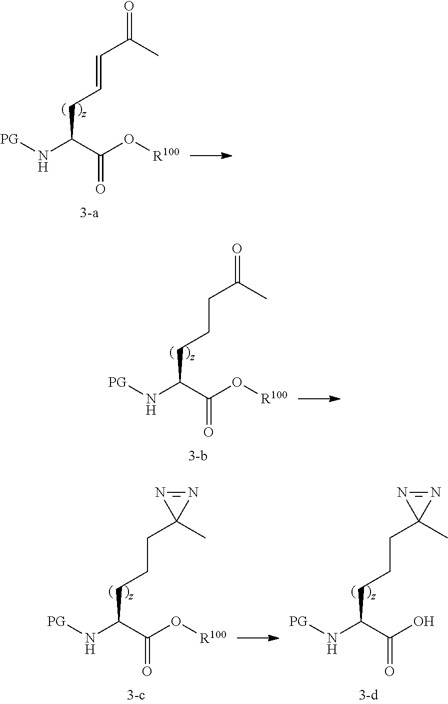

In scheme 1, above, compounds 1-a and 2-a can be obtained from commercial sources. 1-b and 2-b can be provided from 1-a and 2-a, respectively, using standard protecting group chemistry as will be apparent to those skilled in the art. For example, numerous suitable protecting groups are described in T. W. Greene and G. M. Wuts (1999) Protecting Groups in Organic Synthesis, 3rd Edition, Wiley, New York, and references cited therein. 1-c can be provided from 1-b under standard olefin metathesis reaction conditions known in the art. 2-c is provided by selective reduction of 2-b (e.g., AlH(i-Bu)₂). 2-d can be provided from 2-c under Wittig olefination conditions known in the art. Modified amino acids of formula 3-a can be synthesized using similar methods as shown for 2-d, but with varying chain length. For example, long cQFD as used herein is a meditope having an amino acid of formula 3-a, where z is as defined (typically from 3 to 11). Hydrogenation of 3-a provides 3-b. The diazirine moiety can be installed using liquid ammonia followed by hydroxylamine-O-sulfonic acid and oxidation, thus providing 3-c. Ester hydrolysis, and optional reprotection of PG as needed, provides 3-d.

### EXAMPLES

### Example 1: Generation of Antibody-Meditope-Conjugate

Peptide production: All peptides use for experimentation were synthesized and purified by CS Bio, (Menlo Park, Calif.) or at WuXi Apptec Chemistry (Shanghai, China). A CS536XT peptide synthesizer was used for small scale, single batch manufacturing of peptides. Purification was done with an Agilent 1200 HPLC with a Phenomenex Luna C18, 5 μm 250×4.6 mm column, with a flow rate 1 mL/min using a gradient of 1.5% buffer per minute. The buffer system used was comprised of two buffers, where buffer system: A: 0.1% TFA/H₂O and buffer B: 0.08% TFA/acetonitrile. Peptides were lyophilized following purification. Specific photoactivatable amino acids were incorporated into the peptide chain following modification with Fmoc- to permit incorporation using peptide synthesis. Conjugation of toxins to the peptides, including duostatin and MMAD, was performed by Levena Biopharma (San Diego, Calif.). All peptides were provided in DMSO or DMA as lyophilized powders following final analysis and solubilized in DMSO or DMA for photoconjugation.

Materials: Purified meditope-enabled antibody (cetuximab) in PBS (pH 7.2) or 20 mM histidine buffer (pH 7.0); meditope peptide-payload conjugate (meditope with photoactivatable photo-Met at position 10) in DMSO or DMA. Method for laboratory scale production: Mix antibody at 40 microM (e.g. 40 nmole/mL in 10 ml is a total of 400 nmole) or lower with 3× excess meditope-payload conjugate (e.g. 120 microM; or in 10 ml, 1200 nmole total). Incubate with gentle agitation for 30 minutes on ice or at room temperature to allow for noncovalent binding to occur. Distribute 150 microliter per well in 96-well, black, flat bottom polypropylene microplate (Greiner Bio-One, #655209). Irradiate with long range UV (340 nM) 250 mJ/cm². Materials used to perform this activation procedure include UV Crosslinkers, UVP®, Supplier: UVP CL-1000L CROSS LINKER LW 115V which provides Long Range UV, at the desired wavelength of 340 nm. The manufacturer part number is 95-0228-01 (UVP). Buffer exchange is performed on a preparative gel filtration column (for example SRT-10C SEC-300 from Sepax Technologies) to separate unreacted peptide. Fresh (non-irradiated) meditope-payload conjugate is added in 3× molar excess, and mixed for 30 min with agitation at room temperature and irradiation can be repeated in polypropylene plate or UV reactor. Follow with gel filtration to remove unreacted peptide. Additional processing may be done by mixing with fresh meditope-payload, irradiating and performing gel-filtration, the final product should be >90% conjugated. This process may be automated and scaled up using standard automated platforms such as a Biomek Fx fitted with a filtration unit. Scaling this process for larger amounts may be done by using a long range, large scale UV reactor such as those available from Ace (Catalog # Z259462 from Sigma Aldrich). The protocol may be modified to improve the efficiency of covalent conjugation using a filtration device to remove unbound peptide following non-covalent attachment. In this instance, the peptide amount is adjusted to the molar concentration of available meditope-binding sites. For example, if the starting material is 40 microM (nmole/mL) in 10 mL (i.e. 400 nmole) and after the first round 40% of material is conjugated, the amount of available meditope-binding sites is 240 nmole and so at 3× excess the meditope is now 720 nmole (or, in 10 mL, 72 nmole/mL or microM). This procedure can be scaled as required to make larger amounts as needed. Hydrophobic interaction chromatography can be done to separate out the covalently bound material from the unbound material. Profiling by analytical hydrophobic interaction chromatography was done on a Sepax Technologies, Inc. (Newark, Del., USA) Proteomix HIC Butyl-NP5 column (250 mm×10 mm, 2.5 μm) installed on an GE Healthcare Lifesciences ÅKTA pure 25 L system (Pittsburgh, Pa., USA) using a binary gradient of buffer A (20 mM sodium phosphate, 1.5 M Ammonium Sulfate, pH 7.0) and buffer B (20 mM sodium phosphate, 25% v/v isopropanol, pH 7.0) with samples prepared by diluting approximately 100 μg of antibody (PBS) with 0.5 volume of 3 M ammonium sulfate.

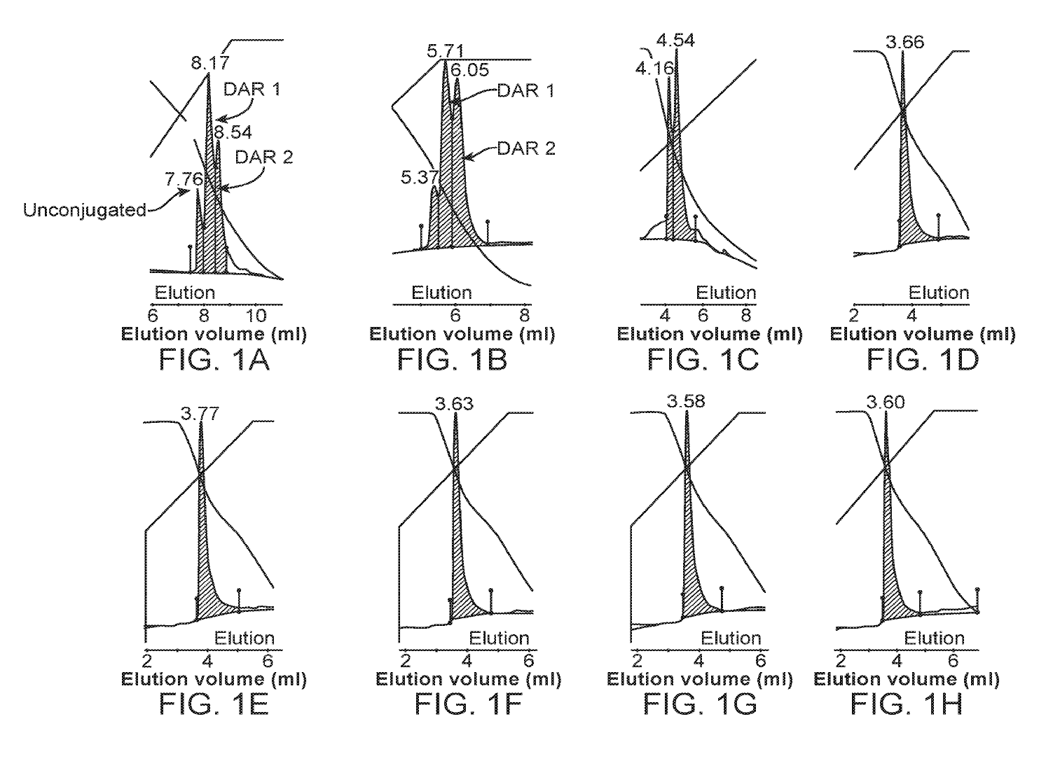

FIG. 1, panels A-H show hydrophobic interaction the presence of covalently bound meditope peptides to the antibody cetuximab. It is contemplated that any meditope enabled antibody would give similar results. Examples shown here show the toxin MMAD and the toxin duostatin conjugated on to meditope peptides containing photoactivatable moieties as described herein covalently bound to cetuximab 183E following noncovalent interaction at room temperature as described herein. The initial reaction causes a 50-60% incorporation of with a mixture of drug antibody ratios (DAR) of 1 and 2 (A). Additional photoactivation with fresh non-irradiated peptide following non-covalent binding at room temperature increases the amount of conjugate, here most obviously noticed as an increase in DAR2 (B). The percentage of conjugate reaches ˜80%. In (C), a second peptide with a different toxin (duostatin) attached to it is able to be covalently locked to the antibody with high efficiency. As shown in D-H, although able to non-covalently interact with and bind in the meditope binding pocket, cetuximab did not efficiently covalent bond with the CQFD peptide with these modifications (D) azido-Phenylalanine at position 6 conjugated to MMAD; (E) azido-Phe 3 MMAD; (F) azido-Phenylalanine at position 10 conjugated to MMAD; (G) benzoyl-Phenylalanine at position 3 conjugated to MMAD and (H) benzoyl-Phenylalanine at position 10 conjugated to MMAD. In this case, amino acid modifications as suggested by Tables 1 and 2 may be made to the antibody that permit the covalent attachment to be efficiently made following non-covalent meditope peptide binding.

Thus, as provided herein, it is contemplated that the meditope-binding cavity can be modified to enhance covalent bonding and thus conjugate formation. Alternatively, or in addition, it is contemplated that the meditope can be modified to enhance covalent bonding.

### Example 2: Method for Measuring Binding Kinetics of Meditope Peptides to Antibodies

The binding kinetics of meditope peptides was evaluated on the Octet Red Platform from Forte Bio by Bio-Layer Interferometry (BLI). Antibodies used in this study have been described in Table 3, and peptides in Table 4. All kinetic experiments were performed on anti-human Fc capture biosensor tips to immobilize the “ligand antibody” (the antibody that contains the meditope peptide binding site). All kinetics experiments were run in 1 or 10x kinetics buffer (10× kinetics buffer: 1% BSA, 0.2% Tween 20, 0.02% sodium azide, in 10x PBS) and 200 to 250 μL of each reagent dilution or buffer were added per well of a black 96 well assay plate. For each kinetics assay the antibody concentration used was 20 μg/mL in 1 to 10× kinetics buffer, the peptide was diluted in 1 to 10× kinetics buffer, either in serial dilutions to establish accurate peptide binding kinetics or at 200 nM to establish relative binding affinity. At the start of the experiment, the biosensor tips were equilibrated for 30 min in 1 to 10× kinetics buffer. The ligand antibody was loaded for 600 seconds onto the biosensor tip, followed by a 300 seconds stabilization/baseline determination in 10× kinetics buffer, after that the antibody loaded biosensors were dipped into the peptide containing solution to allow peptide association for 200 to 600 seconds, followed by a dissociation step for 600 seconds in 10× kinetics buffer. Data analysis was performed with the Octet data analysis software. Buffer background and unspecific binding background were subtracted from binding data using both a reference well and a parallel reference sensor: To measure buffer background, a biosensor loaded with antibody only was incubated in buffer without peptide (=reference well). To measure potential unspecific binding of peptide to biosensor surface or antibody, a reference sensor was loaded with the reference antibody Trastuzumab, which is not meditope enabled and does not contain a meditope-binding site, and incubated with the according peptide (=reference sensor). This reference antibody is distinct from the “enabled Trastuzumab” shown in Table 5 below, which does contain modifications to permit meditope peptide binding. A typical 96 well plate set up for measuring binding kinetics of one peptide in dilution series to one antibody. The shown data are based on the average of three data points measured per peptide-antibody pair at three different peptide concentrations where data range for Kd (M) is 2.65E−05-4.21E−07; data range for kon (1/Ms) is 9.22E+02-4.01E+07; and the data range for kdis(1/s) is 1.43E+01-8.50E−03. The binding data is shown in Table 5, below.

TABLE 5
Kd (M)

| | | Meditope | | | Meditope | Meditope |
|---|---|---|---|---|---|---|
| | | enabled | Cetuximab- | Cetuximab | enabled | enabled |
| Peptide | | Trastuzumab | V2 | (I83E) | Gemtuzumab | Lintuzumab |
| Peptide 5 | photo-Met10- | +++ | + | ++++ | ++++ | +++ |
| | cQFD-MMAD | | | | | |
| Peptide 6 | photo-Met9- | + | + | + | + | + |
| | cQFD-MMAD | | | | | |
| Peptide 8 | photo-Met10- | + | + | ++ | ++ | + |
| | cQFD-Duostatin | | | | | |
| Peptide 10 | azido-Phe3- | ++++ | + | +++ | +++ | ++++ |
| | cQFD-MMAD | | | | | |
| Peptide 11 | azido-Phe10- | + | + | + | + | + |
| | cQFD-MMAD | | | | | |
| Peptide 12 | benzoyl-Phe3- | + | + | + | + | + |
| | cQFD-MMAD | | | | | |
| Peptide 13 | benzoyl-Phe10- | + | + | + | + | + |
| | cQFD-MMAD | | | | | |

kon(1/Ms)

| | | Meditope | | | Meditope | Meditope |
|---|---|---|---|---|---|---|
| | | enabled | Cetuximab- | Cetuximab | enabled | enabled |
| Peptide | | Trastuzumab | V2 | (I83E) | Gemtuzumab | Lintuzumab |
| Peptide 5 | photo-Met10- | ++++ | + | ++++ | ++++ | ++ |
| | cQFD-MMAD | | | | | |
| Peptide 6 | photo-Met9- | + | + | + | + | + |
| | cQFD-MMAD | | | | | |
| Peptide 8 | photo-Met10-cQFD- | + | + | +++ | ++ | + |
| | Duostatin | | | | | |
| Peptide 10 | azido-Phe3- | ++++ | + | ++++ | +++ | ++++ |
| | cQFD-MMAD | | | | | |
| Peptide 11 | azido-Phe10- | + | + | + | + | + |
| | cQFD-MMAD | | | | | |
| Peptide 12 | benzoyl-Phe3- | + | + | + | + | + |
| | cQFD-MMAD | | | | | |
| Peptide 13 | benzoyl-Phe10- | + | + | + | + | + |
| | cQFD-MMAD | | | | | |

kdis(1/s)

| | | Meditope | | | Meditope | Meditope |
|---|---|---|---|---|---|---|
| | | enabled | Cetuximab- | Cetuximab | enabled | enabled |
| Peptide | | Trastuzumab | V2 | (I83E) | Gemtuzumab | Lintuzumab |
| Peptide 5 | photo-Met10- | ++++ | + | ++++ | +++ | +++ |
| | cQFD-MMAD | | | | | |
| Peptide 6 | photo-Met9- | + | + | + | + | + |
| | cQFD-MMAD | | | | | |
| Peptide 8 | photo-Met10-cQFD- | + | + | ++++ | ++ | + |
| | Duostatin | | | | | |
| Peptide 10 | azido-Phe3- | ++++ | + | ++++ | ++++ | ++++ |
| | cQFD-MMAD | | | | | |
| Peptide 11 | azido-Phe10- | + | + | + | + | + |
| | cQFD-MMAD | | | | | |
| Peptide 12 | benzoyl-Phe3- | + | + | + | + | + |
| | cQFD-MMAD | | | | | |
| Peptide 13 | benzoyl-Phe10- | + | + | + | + | + |
| | cQFD-MMAD | | | | | |

As shown above, peptides with photo-Met10 with MMAD has higher affinity to meditope enabled antibodies, compared to photo-Met 9. Peptides with azido-Phe3 with MMAD has higher affinity to meditope enabled antibodies, compared to azido-Phe10, benzoyl-Phe3, and benzoyl-Phe10. Further, the results showed that peptides with photo-Met10 with MMAD has higher affinity compared to photo-Met10 with Duostatin. As such, the position of photoreactive functional groups in the meditope sequence could influence the binding kinetics of meditope enabled antibodies and highlighted which specific amino acids important for meditope:antibody interaction. Structural modeling and analyses of the interactions allowed us to optimize the photoreactive conjugation between photoreactive functional groups and meditope enabled antibodies. It is further contemplated that meditopes with different payloads may have different binding kinetics to the antibodies.

### Example 3: Cell Killing with Antibody-Meditope Conjugates with Photomet 10 Peptide

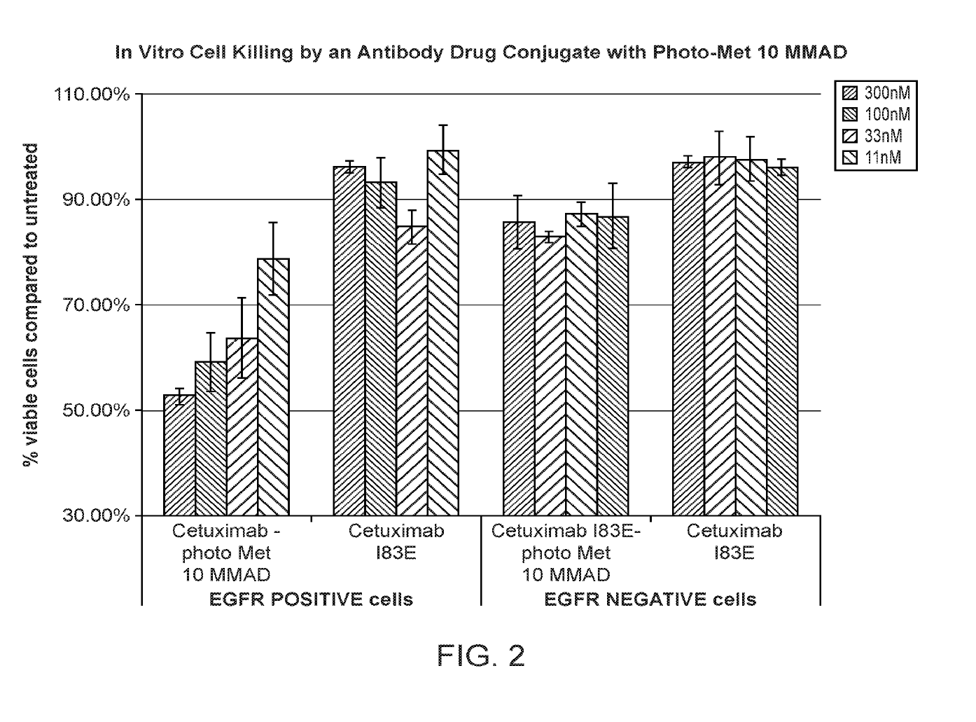

A cell killing experiment was performed using iAlamar Blue according to manufacturer's instructions. Briefly, SW48, an epithelial cell line described as being from colon and possessing the qualities of a colorectal adenocarcinoma, including epidermal growth factor (EGFR) mutations (ATCC catalog # CCL-231) and HL60, a non-colorectal, myeloid cell line with no EGFR expression or mutation were tested for sensitivity to an anti-EGFR antibody coupled to a toxic auristatin (MMAD) molecule via a covalently linking meditope peptide with a photo activatable methionine (diazirine) at position 10 of the CQFD peptide (described above). Cells were plates at 20,000 cells per well in 10% fetal bovine serum and complete media. Cells were treated with antibody alone or antibody drug conjugate with photo-Met 10 peptide-MMAD. The antibody used was Cetuximab with an I83E mutation. Cells were incubated at 37 C in 5% CO₂ for 48 hours and then Alamar Blue was plated 1:10 in all wells (ThermoFisher, catalog number DAL-1025) and read 4 hours post plating using a Molecular Devices Spectramax equipped with a fluorescent reading capability at the manufacturer's suggested excitation and emission spectra of 530/590. FIG. 2 shows the results of this experiment, where only cells that express EGFR are effectively killed by the EGFR antibody drug conjugate. This further shows the stability of the covalent interaction, as free peptide linked to toxin would cause toxicity in both cell lines, but only causes toxicity in the EGFR targeted cell line.

### Example 4: In Vivo Antibody-Meditope Conjugates with Photomet 10 Peptide

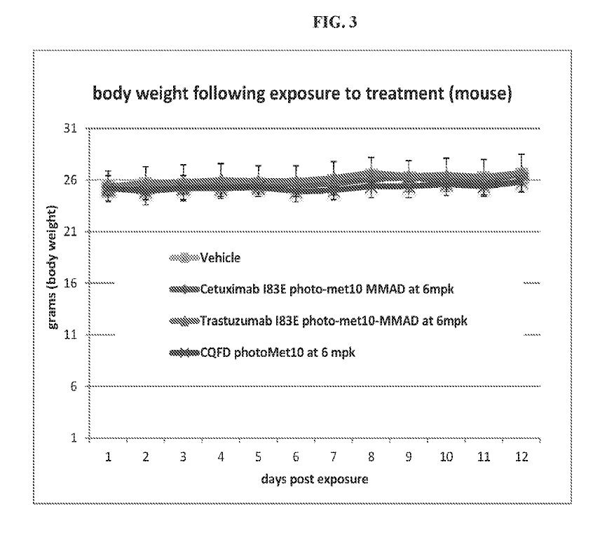

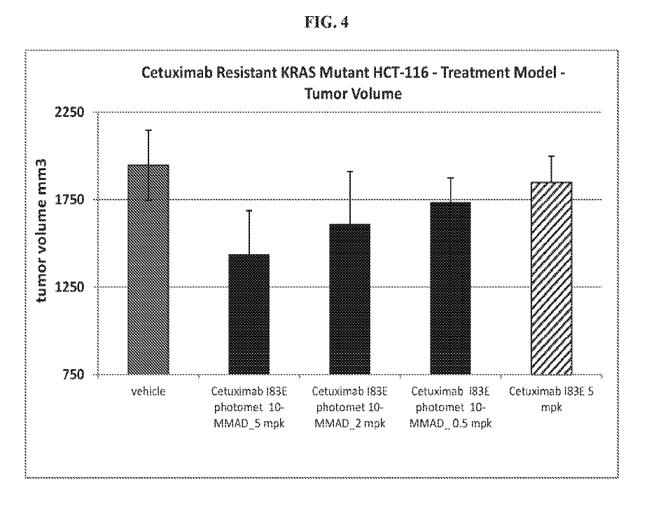

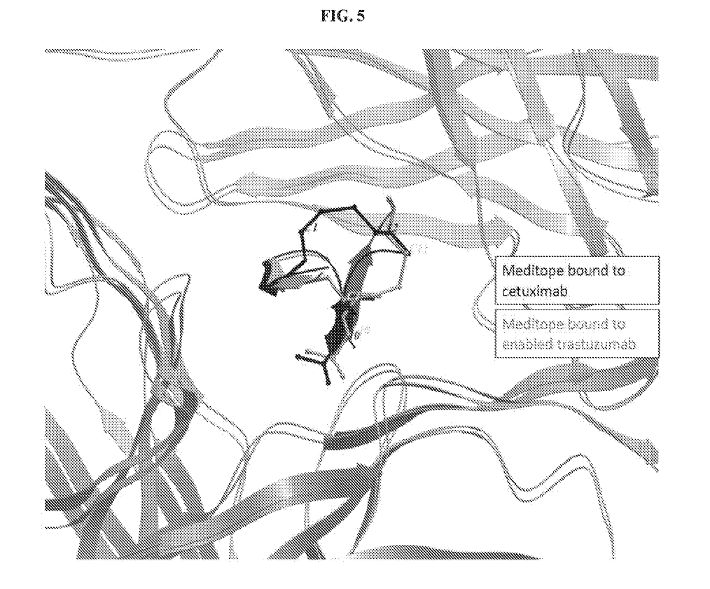

A test of antibodies covalently bound to meditope peptides via photoactivation of specific photoactivatable amino acids as described herein to determine the stability of the interaction was done by first testing mice for weight loss following exposure to the covalently bound antibodies. A second test was done to see if targeted tumor killing could be seen with conjugates targeted to specific antigens. In this case, a xenograft comprised of EGFR positive human cancer cells was tested for reduction and or repression of tumor volume in an aggressive tumor model of HCT-16 cells in NOD/SCID mice. In this case, an anti-EGFR antibody conjugated in a stable covalent fashion to a cytotoxin is expected to show some reduction and or repression of tumor growth (FIG. 4). A non-GLP single dose toxicity study of compounds was done in BALB/c mice. Dosing was done Day 0. The animals were housed three in a cage with two cages per group for a total of 6 animals per test treatment. Animals were ear-tagged for identification. Measurements were taken daily throughout the study including body weight (FIG. 3) and clinical symptoms. At day 11 organs were taken and weighed and a complete blood count (CBC) was performed. Organs harvested and weighed included liver, lungs, heart, spleen, kidneys, bladder, and a section of skin from the abdominal region (Table 4). All organs were placed in 10% neutral buffered formalin. Terminal bleeds were made via a cardiac puncture. Approximately 100 μL of whole blood was collected in EDTA plasma tubes and kept on ice for subsequent CBC analysis. CBC analysis was done within 24 hours of collection. Animals received the compounds in 200-500 microliter volume via an intraperitoneal route of administration. All IP injections were performed over the course of two minutes. Table 6 below shows organ weights for two photoactivatable linked peptides with the photoactivatable residue at position 10 conjugated to MMAD. No difference was noted in organ weights, suggesting a stable conjugation and no free peptide. Free peptide (not linked to antibody) with the photoactivated conjugation was also injected and also did not cause any organ weight changes, suggesting no overt toxicity to the peptide alone.

TABLE 6

| | | | Cetuximab | Trastuzumab | CQFD |
|---|---|---|---|---|---|
| | Organ | | I83E photo- | I83E photo- | peptide |
| | Weights | | met10 | met10 | with photo |
| | at | | MMAD | MMAD at | Met10 |
| Tissue | Day 11 | Vehicle | at 6 mpk | 6 mpk | at 6 mpk |
| heart | Mean | 137.20 | 120.60 | 133.80 | 125.10 |
| | SD | 15.30 | 12.50 | 14.30 | 13.10 |
| lungs | Mean | 181.50 | 190.40 | 183.60 | 184.30 |
| | SD | 9.30 | 11.50 | 9.90 | 14.20 |
| liver | Mean | 1419.00 | 1443.00 | 1400.00 | 1373.00 |
| | SD | 218.60 | 124.60 | 129.80 | 90.30 |
| spleen | Mean | 106.80 | 114.40 | 120.00 | 110.80 |
| | SD | 17.50 | 18.00 | 33.40 | 19.40 |
| kidney | Mean | 444.70 | 415.40 | 416.30 | 396.60 |
| | SD | 71.40 | 34.80 | 36.10 | 21.10 |
| bladder | Mean | 27.30 | 34.80 | 35.20 | 39.10 |

### Example 5: Binding Kinetics of EGFR to Cetuximab Variants with and without Peptide 8

The binding kinetics was tested to evaluated the impact of meditopes as disclosed herein (Peptide 8) on antigen (EGFR) recognition of Cetuximab antibody variants.

In this study, Cetuximab variants I83E, variant 2 (V2), variant 3 (V3), with and without Peptide 8, were tested. Variant 2 of Cetuximab (Cetuximab V2) has modifications of LC I83E, K103R, E165D and HC E154S, Q111K. Variant 3 of Cetuximab (Cetuximab V3) has modifications of LC I83E, K103Q, E165P and HC Q111 S, E154D. In addition, an Cetuximab I83E-ADC (Cetuximab I83E-Duostatin) with Peptide 8 was also tested. The kinetic experiments were performed using the Octet Red 96 as described previously. The binding data is shown in Table 7, below.

TABLE 7

| Antibody | KD (M) | StDev | kon(1/Ms) | StDev | kdis(1/s) | StDev |
|---|---|---|---|---|---|---|
| Cetuximab_I83E | 3.0E−09 | 2.2E−11 | 3.7E+05 | 9.2E+02 | 1.0E−03 | 1.8E−06 |
| Cetuximab_I83E + Peptide 8 | 4.3E−09 | 3.7E−11 | 4.7E+05 | 1.2E+04 | 1.5E−03 | 3.0E−06 |
| Cetuximab_I83E − Duostatin + | 2.6E−09 | 1.7E−11 | 4.6E+05 | 1.0E+03 | 1.0E−03 | 1.2E−06 |
| Peptide 8 | | | | | | |
| Cetuximab_V2 | 2.6E−09 | 1.7E−11 | 4.7E+05 | 8.4E+02 | 1.1E−03 | 1.2E−06 |
| Cetuximab_V2 + Peptide 8 | 3.1E−09 | 2.6E−11 | 5.9E+05 | 4.8E+03 | 1.3E−03 | 9.1E−07 |
| Cetuximab_V3 | 3.1E−09 | 2.3E−11 | 4.0E+05 | 1.1E+03 | 1.1E−03 | 1.7E−06 |
| Cetuximab_V3 + Peptide 8 | 3.5E−09 | 2.9E−11 | 5.6E+05 | 4.2E+03 | 1.4E−03 | 9.4E−07 |

The results showed that the presence of payload (Peptide 8) did not influence antigen recognition of Cetuximab antibody variants and that specific modifications in the meditope binding region of the antibody did not impede the antibody from binding antigen (EGFR) in a consistent manner.

### Example 6: Binding Kinetics of Peptide 8 and Peptide 19 to Cetuximab Variants

Further, the binding kinetics of photo-peptides (Peptide 8) to bind to Cetuximab variants was evaluated. Variants of Cetuximab tested were Cetuximab_I183E, Cetuximab_V2, and Cetuximab_V3. The binding data was shown in Table 8, below.

TABLE 8

| Antibody | KD (M) | StDev | kon(1/Ms) | StDev | kdis(1/s) | StDev |
|---|---|---|---|---|---|---|
| Cetuximab_I83E | 6.1E−08 | 1.5E−09 | 3.9E+04 | 2.7E+03 | 1.2E−03 | 5.1E−06 |
| Cetuximab_V2 | 1.4E−05 | 1.4E−02 | 1.3E+05 | 3.2E+06 | 1.4E−01 | 6.6E−02 |
| Cetuximab_V3 | 1.1E−07 | 3.7E−07 | 8.5E+04 | 6.6E+03 | 5.3E−03 | 5.6E−06 |

As shown in Table 8, variants of Cetuximab had different binding kinetics. The fastest on-rate of Peptide 8 to Cetuximab variants was Cetuximab_V2>Cetuximab_V3>Cetuximab_I183E. The slowest off-rate was Cetuximab_I183E>Cetuximab_V3>Cetuximab_V2. The results showed that specific amino acid changes in the meditope binding pocket of the antibody impacted the binding affinity of the meditope. It is further contemplated that in Cetuximab_V2 and Cetuximab_V23, LC E165 modification perturbed binding of photoreactive peptides. The modification K103R (in Cetuximab_V2) and K103Q (Cetuximab_V3) could also affect binding.

Further, the binding kinetics of Peptide 19 with elongated side chain at position 10 to the antibodies was tested. The antibodies tested were Cetuximab 183E, Cetuximab V2, Cetuximab V3, and meditope enabled Clivatuzumab. The binding data was shown in Table 9, below.

TABLE 9

| Antibody | KD (M) | kon(1/Ms) | kdis(1/s) |
|---|---|---|---|
| Cetuximab_I83E | 2.78E−08 | 1.44E+05 | 3.62E−03 |
| Cetuximab_V2 | 7.13E−08 | 6.95E+04 | 4.95E−03 |
| Cetuximab_V3 | 4.345E−08 | 2.97E+05 | 1.06E−02 |
| Meditope enabled Clivatuzumab | no binding | no binding | no binding |
| MBI_CD19_1; (SAR3419) | no binding | no binding | no binding |
| MBI_CD19_2; (MOR208) | 3.38E−08 | 1.02E+05 | 3.46E−03 |
| Meditope enabled Ocaratuzumab | no binding | no binding | no binding |
| Pmab | 3.87E−07 | 7.08E+04 | 2.57E−02 |
| Tmab | 1.20E−07 | 4.85E+04 | 5.77E−03 |

The results showed that the elongated side chain at position 10 in Peptide 19 improved binding kinetics of the meditope to photo-optimized antibodies. Thus, modification of the side chain in the meditope may change the binding affinity to binding pocket of the antibodies.

### Example 7: Binding Kinetics and Affinity of Photo Enabled Meditope with Payload to Meditope Enabled Antibodies

The binding kinetics of long-cQFD-photoMet10 with payload/toxin to Cetuximab I83E was tested to evaluate influence of payload on the binding kinetics and affinity. The payloads in this study were MMAD, Duostatin, DM1, Duocarmycin, or Amanitin. In all cases, successful and straightforward conjugation of toxins to meditope peptides was noted. The binding data of toxin conjugated meditopes to Cetuximab was shown in Table 10, below.

TABLE 10

| Peptide | Payload | KD (M) | kon(1/Ms) | kdis(1/s) |
|---|---|---|---|---|
| Peptide 5 (Lot 1) | MMAD | 3.88E−07 | 3.95E+04 | 1.37E−03 |
| Peptide 5 (Lot 2) | MMAD | 1.11E−06 | 3.03E+04 | 1.38E−03 |
| Peptide 8 | Duostatin | 1.28E−06 | 6.44E+03 | 1.64E−03 |
| Peptide 15 | DM1 | 1.10E−06 | 3.05E+04 | 1.81E−03 |
| Peptide 16 | Duocarmycin | 9.70E−07 | 1.55E−03 | 3.82E−03 |
| Peptide 18 | Amanitin | 4.16E−06 | 2.87E+03 | 1.79E−03 |

The results showed no or minimal influence of different payload chemistry on binding affinity and kinetics of long-cQFD-photo-Met10 to Cetuximab I83E.

The binding kinetics of peptide 25 (long-cQFD-photoMet10, 5-carbon chain at position 10, with no toxin) to photo-optimized antibodies was tested. The binding data is shown in Table 11, below.

TABLE 11

| Peptide | Antibody | KD (M) | kon(1/Ms) | kdis(1/s) |
|---|---|---|---|---|
| Peptide 25 | Cetuximab I83E | 7.63E−09 | 6.60E+05 | 4.81E−03 |
| (without toxin) | Cetuximab V3 | 7.57E−09 | 1.20E+06 | 8.86E−03 |
| | MBI_CD19_2; | 1.32E−08 | 3.84E+05 | 4.80E−03 |
| | (MOR208) | | | |
| | Pmab | 7.94E−08 | 4.14E+05 | 3.25E−02 |
| | Tmab | 2.86E−08 | 1.85E+05 | 5.11E−03 |
| Peptide 8 (with | Cetuximab I83E | 2.78E−08 | 1.44E+05 | 3.62E−03 |
| Duostatin) | Cetuximab V3 | 4.345E−08 | 2.97E+05 | 1.06E−02 |
| | Meditope | no binding | no binding | no binding |
| | enabled | | | |
| | Clivatuzumab | | | |
| | Cetuximab V2 | 7.13E−08 | 6.95E+04 | 4.95E−03 |
| | MBI_CD19_1; | no binding | no binding | no binding |
| | (SAR3419) | | | |
| | MBI_CD19_2; | 3.38E−08 | 1.02E+05 | 3.46E−03 |
| | (MOR208) | | | |
| | Meditope | no binding | no binding | no binding |
| | enabled | | | |
| | Ocaratuzumab | | | |
| | Pmab | 3.87E−07 | 7.08E+04 | 2.57E−02 |
| | Tmab | 1.20E−07 | 4.85E+04 | 5.77E−03 |

The results showed that meditope peptide 25 can bind photo-optimized antibodies Pmab and Tmab regardless of payload toxin.

Further, the binding kinetics of long-cQFD-photoMet10 peptide (5-chain at position 10), with or without payload was tested. Peptide 19 (with Duostatin) and Peptide 25 (without toxin) were used in this study to evaluate the binding to antibodies. The results showed that payloads such as Duostatin could actually improve the “on rate” (Kon) and overall KD for the long-cQFD-photoMet10 peptide (5-chain at position 10) to Tmab (photo-optimized meditope enabled Trastuzumab variant 2). The binding data is shown in Table 12, below.

TABLE 12

| Peptide | Antibody | KD (M) | kon(1/Ms) | kdis(1/s) |
|---|---|---|---|---|
| Peptide 25 | Cetuximab I83E | 7.63E−09 | 6.60E+05 | 4.81E−03 |
| Peptide 19 | | 7.59E−09 | 2.89E+05 | 2.16E−03 |
| Peptide 25 | Tmab | 7.63E−09 | 6.60E+05 | 4.81E−03 |
| Peptide 19 | | 3.38E−08 | 1.28E+05 | 4.26E−03 |
| Peptide 25 | Pmab | 7.94E−08 | 4.14E+05 | 3.25E−02 |
| Peptide 19 | | 3.58E−08 | 3.56E+05 | 1.27E−02 |

A further experiment was conducted to evaluate the binding kinetics of Peptide 25 (without payload) and Peptide 19 (with Duostatin) to Cetuximab 183E, Tmab or Pmab (photo-optimized meditope enabled Panitumumab variant 2). The binding data is shown in Table 13, below.

TABLE 13

| Peptide | Antibody | KD (M) | kon(1/Ms) | kdis(1/s) |
|---|---|---|---|---|
| Peptide 25 | Cetuximab I83E | 1.00E−08 | 6.25E+05 | 6.29E−03 |
| (no toxin) | Pmab | 4.77E−07 | 5.39E+05 | 3.50E−02 |
| | Tmab | 9.61E−07 | 9.32E+04 | 1.05E−02 |
| Peptide 19 | Cetuximab I83E | 1.18E−08 | 2.87E+05 | 2.81E−03 |
| (with 1 | Pmab | 4.6715E−08 | 2.69E+05 | 1.26E−02 |
| Duostatin per | Tmab | 4.2795E−08 | 1.01E+05 | 4.29E−03 |
| peptide, DAR 2) | | | | |

The results show that addition of one Duostatin to long-cQFD-photo-Met10 (5-chain) improved binding of the peptides to enabled antibodies, possibly due to hydrophobicity of the chosen toxin. Further, addition of one Duostation to long-cQFD-photoMet10 (5-chain) had minimal effect on affinity (Kd), on-rate (Kon) and off-rate (K-off) for Cetuximab 183E. For binding to Tmab and Pmab, addition of one Duostatin to long-cQFD-photoMet10 (5-chain) increased affinity (Kd) and decreased the off-rate (slower off-rate).

Further, the binding kinetics study was conducted to evaluate the impact of toxin payload on binding kinetics of a difunctionalized (i.e., two toxins) meditope long-cQFD-photoMet10 peptide (4-chain at position 10) DAR of 2 versus DAR of 4 to antibodies. In this study, Peptide 24 with no toxin, Peptide 8 with 1 duostatin, and Peptide 21 with 2 duostatins were used to test the binding kinetics to Cetuximab 183E, Pmab, and Tmab. The kinetic experiments were performed using the Octet Red 96 as described previously. The results showed that the meditopes having more than one active agent bind to the meditope enabled antibodies. The payload to the long-cQFD-photoMet10 peptide (4-chain at position 10) may impact the binding kinetics to the antibodies. The binding data is shown in Table 14, below.

TABLE 14

| Peptide | Antibody | KD (M) | kon(1/Ms) | kdis(1/s) |
|---|---|---|---|---|
| Peptide 24 (no | Cetuximab I83E | 5.00E−09 | 4.36E+05 | 2.06E−03 |
| toxin) | Pmab | 6.35E−09 | 2.01E+05 | 1.18E−03 |
| | Tmab | 2.55E−07 | 5.40E+04 | 2.66E−03 |
| Peptide 8, with 1 | Cetuximab I83E | 1.75E−08 | 2.03E+05 | 3.31 − 03 |
| Duostatin per | Pmab | 4.257E−08 | 1.45E+05 | 6.15E−03 |
| peptide (DAR 2) | Tmab | 5.9625E−08 | 4.23E+05 | 2.52E−02 |
| Peptide 21, with | Cetuximab I83E | 3.71E−06 | 7.04E+03 | 1.10E−02 |
| 2 Duostatins per | Pmab | 3.45E−06 | 6.77E+05 | 2.98E−02 |
| peptide (DAR 4) | Tmab | 6.91E−06 | 6.70E+04 | 9.00E−03 |

### Example 8: UV Conjugation of Tmab with Photo Enabled Meditopes

The following evaluated UV conjugation of Tmab with photo enabled peptides using Hydrophobic interaction chromatography (HIC). Unless optimized, antibodies could not photo conjugate properly. An optimized version of Trastuzumab (Tmab) was used to evaluate the conjugation with photo peptides. The photo peptides used in this example included Peptide 8, Peptide 21, and Peptide 26.

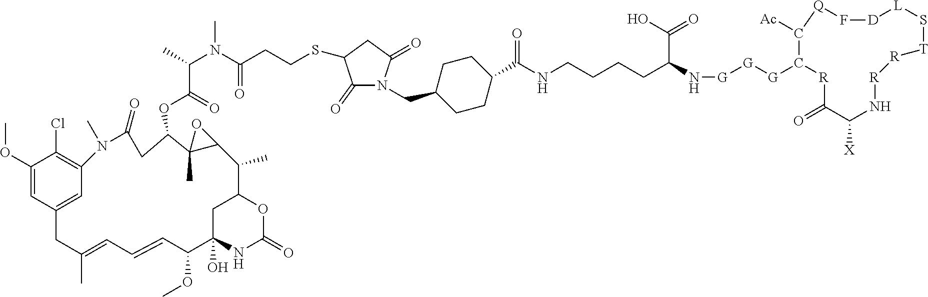

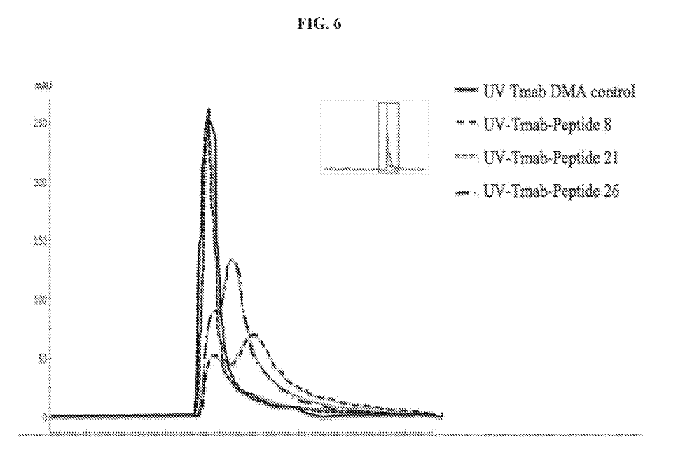

Tmab was mixed with dimethoxyamphetamine (DMA) for the control. The results, as shown in FIG. 6, demonstrated that Tmab conjugated with Peptide 8 and Peptide 26, but not Peptide 21. It was shown that Tmab had highly efficient and differential photo-conjugation to different meditopes.

### Example 9: Binding Kinetics of Cross-Linking Meditopes with Elongated Chain Length at Position 10

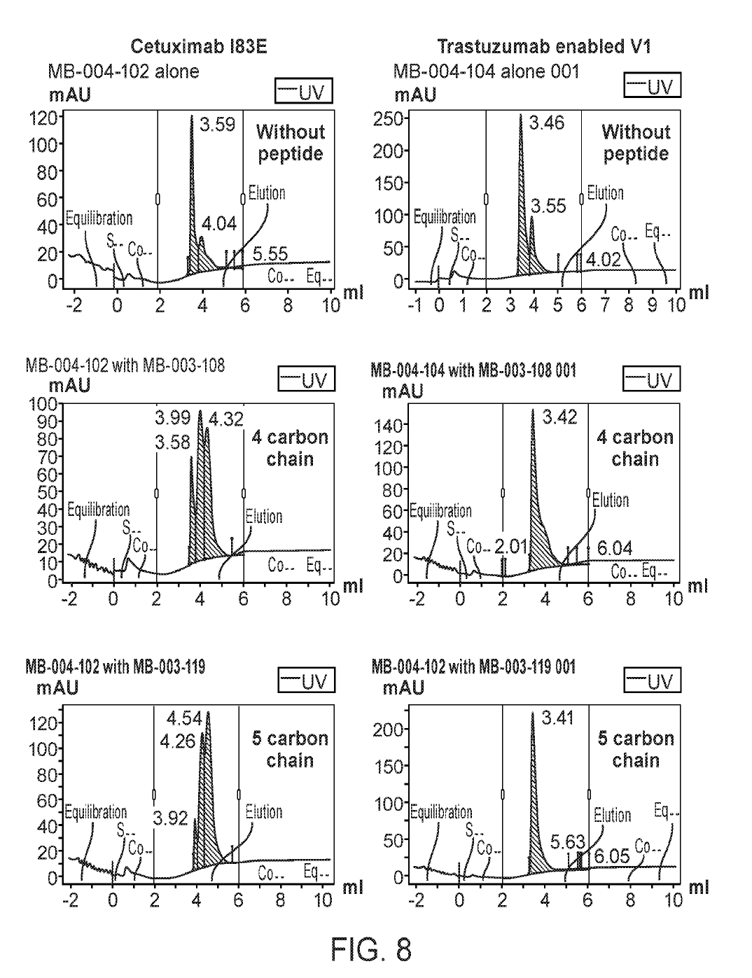

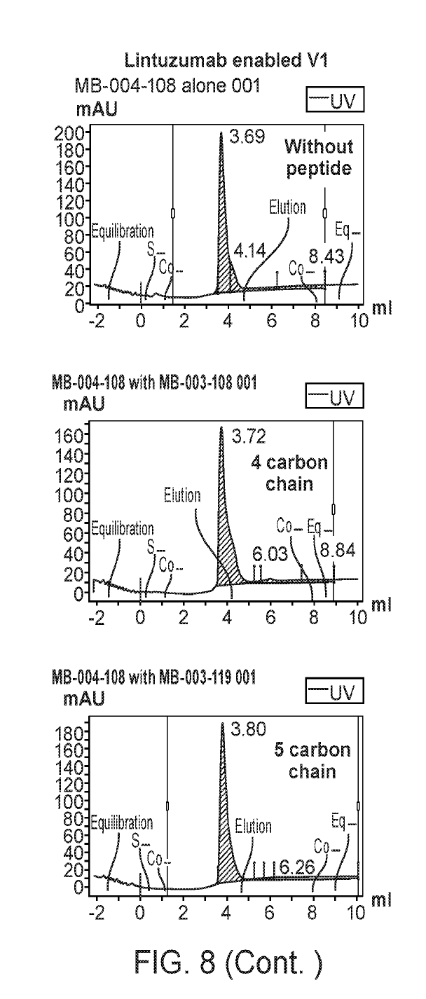

HIC was performed to test affinity and binding kinetics of cross-linking meditopes with elongated chain length at position 10 with 5 carbon chain and 4 carbon chain to Cetuximab 183E, Tras V1, and Lintuzumab. The results showed that meditopes with 5 carbon chain has a higher affinity to Cetuximab 183E and Tras V1 (FIG. 8).

### Example 10: Cetuximab I83E Conjugated with Meditopes with Different DAR

UV-conjugation of Cetuximab 183E with Peptide 8 and Peptide 26 was performed and HIC was performed to test the binding kinetics. The results showed that Peptide 8 conjugated 100% with variable DAR (DAR1 and DAR2), while Peptide 26 conjugated 100% with single species DAR (DAR1).

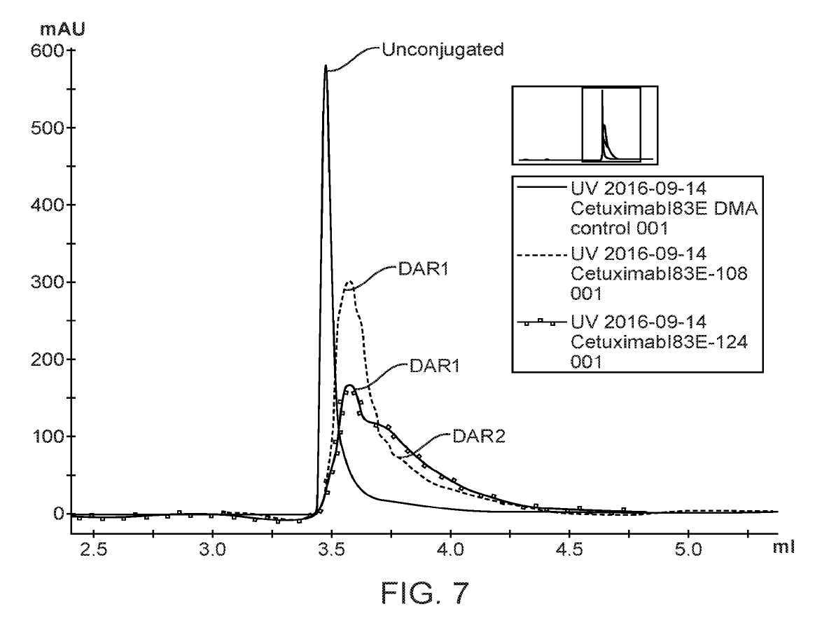

The results are shown in FIG. 7, which demonstrates that different meditopes can be designed to selectively produce different DAR.

### Example 11: Binding of Meditopes to Antibodies

The binding kinetics of photo enabled meditopes to antibodies (e.g., meditope enabled Trastuzumab, T-mab, and P-mab) was tested. The binding data is shown in Table 14, below.

TABLE 14

| Antibody | Peptide | KD (M) | kon(1/Ms) | kdis(1/s) |
|---|---|---|---|---|
| Meditope enabled | Peptide 5 | 9.207E−08 | 6.62E+04 | 6.16E−03 |
| Trastuzumab | Peptide 8 | 2.135E−07 | 5.34E+04 | 9.90E−03 |
| | Peptide 19 | 1.4905E−07 | 9.53E+04 | 1.38E−02 |
| Tmab | Peptide 5 | 7.1725E−08 | 5.09E+04 | 3.50E−03 |
| | Peptide 8 | 4.257E−08 | 1.45E+05 | 6.15E−03 |
| | Peptide 19 | 4.2795E−08 | 1.01E+05 | 4.29E−03 |
| Pmab | Peptide 5 | 5.455E−08 | 1.79E+05 | 9.54E−03 |
| | Peptide 8 | 5.9625E−08 | 4.23E+05 | 2.52E−02 |
| | Peptide 19 | 4.6715E−08 | 2.69E+05 | 1.26E−02 |

The results confirmed binding of photo enabled peptides to the photo optimized antibodies Tmab and Pmab. Further, the binding kinetics of photo enabled meditopes to various antibodies at different concentrations was tested. The binding data is shown in Table 15, below.

TABLE 15

| | | Conc. | | KD | | kon | | kdis |
|---|---|---|---|---|---|---|---|---|
| Antibody | Peptide | (nM) | KD (M) | Error | kon(1/Ms) | Error | kdis(1/s) | Error |
| Meditope | Peptide | 200 | 8.17E−05 | 1.78E−02 | 3.97E+02 | 8.64E+04 | 3.24E−02 | 1.08E−02 |
| enabled | 5 | 100 | 2.69E−06 | 5.30E−06 | 9.31E+03 | 1.83E+04 | 2.51E−02 | 1.07E−03 |
| Trastuzumab | | 50 | 2.35E−06 | 2.26E−05 | 4.17E+03 | 4.01E+04 | 9.80E−03 | 8.07E−04 |
| | Peptide | 200 | 1.91E−11 | 2.92E−02 | 3.59E+08 | 5.48E+17 | 6.85E−03 | 2.06E−04 |
| | 6 | 100 | 2.51E−09 | 5.52E−10 | 3.65E+06 | 7.93E+05 | 9.16E−03 | 3.17E−04 |
| | | 50 | <1.0E−12 | | 1.82E+48 | | 2.08E−03 | 1.09E−04 |
| | Peptide | 200 | 3.02E−05 | 5.71E−04 | 5.37E+02 | 1.01E+04 | 1.62E−02 | 1.06E−03 |
| | 10 | 100 | 1.81E−05 | 4.22E−04 | 2.66E+02 | 6.21E+03 | 4.80E−03 | 1.64E−04 |
| | | 50 | <1.0E−12 | 1.36E−05 | 8.16E+02 | 1.58E+06 | <1.0E−07 | |
| | Peptide | 200 | 7.33E−08 | 1.47E−08 | 2.86E+05 | 5.40E+04 | 2.10E−02 | 1.43E−03 |
| | 12 | 100 | 1.22E−09 | 3.26E−10 | 9.47E+06 | 2.51E+06 | 1.16E−02 | 3.35E−04 |
| | | 50 | <1.0E−12 | <1.0E−12 | 1.16E+97 | 0.00E+00 | 1.33E−02 | 9.13E−04 |
| Cetuximab | Peptide | 333 | <1.0E−12 | 1.22E−10 | 6.91E+04 | 1.78E+03 | <1.0E−07 | |
| | 22 | 111 | <1.0E−12 | 2.07E−11 | 2.05E+05 | 2.66E+03 | <1.0E−07 | |
| | | 37 | 1.10E−10 | 7.31E−12 | 2.85E+05 | 1.30E+03 | 3.14E−05 | 2.08E−06 |
| Meditope | Peptide | 333 | 2.69E−09 | 7.45E−10 | 1.12E+06 | 3.08E+05 | 3.02E−03 | 8.42E−05 |
| enabled | 22 | 111 | 3.16E−06 | 1.22E−03 | 2.36E+05 | 9.10E+07 | 7.44E−01 | 6.94E+00 |
| Clivatuzumab | | 37 | 4.53E+12 | 5.50E+39 | 7.13E+05 | 8.52E+32 | 3.23E+18 | 7.00E+44 |
| Cetuximab V3 | Peptide | 333 | 2.44E−09 | 7.28E−11 | 4.23E+05 | 1.24E+04 | 1.03E−03 | 6.14E−06 |
| | 22 | 111 | 2.18E−09 | 2.50E−11 | 4.57E+05 | 4.97E+03 | 9.93E−04 | 3.63E−06 |
| | | 37 | 1.90E−09 | 1.34E−11 | 4.66E+05 | 2.87E+03 | 8.86E−04 | 3.07E−06 |
| MBI_CD19_1; | Peptide | 333 | 5.23E−09 | 3.53E−10 | 2.16E+04 | 3.27E+02 | 1.13E−04 | 7.43E−06 |
| (SAR3419) | 22 | 111 | <1.0E−12 | 5.55E−10 | 2.10E+04 | 8.63E+02 | <1.0E−07 | |
| | | 37 | <1.0E−12 | 8.19E−10 | 3.11E+04 | 5.15E+03 | <1.0E−07 | |
| MBI_CD19_2; | Peptide | 333 | <1.0E−12 | 1.16E−10 | 6.81E+04 | 1.64E+03 | <1.0E−07 | |
| (MOR208) | 22 | 111 | 4.52E−10 | 9.27E−12 | 2.30E+05 | 1.45E+03 | 1.04E−04 | 2.03E−06 |
| | | 37 | <1.0E−12 | 8.57E−12 | 2.19E+05 | 8.58E+02 | <1.0E−07 | |
| Meditope | Peptide | 333 | <1.0E−12 | 7.44E−09 | 4.30E+03 | 7.35E+02 | <1.0E−07 | |
| enabled | 22 | 111 | <1.0E−12 | 1.92E−09 | 1.85E+04 | 2.56E+03 | <1.0E−07 | |
| Ocaratuzumab | | 37 | <1.0E−12 | 7.46E−11 | 5.08E+05 | 5.36E+04 | <1.0E−07 | |
| Pmab | Peptide | 333 | 1.83E−08 | 6.72E−10 | 1.66E+05 | 5.90E+03 | 3.03E−03 | 2.61E−05 |
| | 22 | 111 | 8.59E−09 | 2.92E−10 | 2.36E+05 | 7.61E+03 | 2.02E−03 | 2.13E−05 |
| | | 37 | 7.33E−09 | 1.97E−10 | 2.42E+05 | 5.97E+03 | 1.77E−03 | 1.86E−05 |
| Tmab | Peptide | 333 | 1.63E−09 | 3.81E−11 | 7.49E+04 | 6.11E+02 | 1.22E−04 | 2.67E−06 |
| | 22 | 111 | 1.88E−09 | 2.18E−11 | 9.77E+04 | 4.14E+02 | 1.83E−04 | 1.98E−06 |
| | | 37 | 2.62E−09 | 4.14E−11 | 9.87E+04 | 8.35E+02 | 2.59E−04 | 3.45E−06 |
| Meditope | Peptide | 1000 | 6.45E−08 | 2.58E−09 | 1.77E+05 | 6.88E+03 | 1.14E−02 | 1.04E−04 |
| enabled | 8 | 333 | 6.57E−08 | 1.54E−09 | 2.17E+05 | 4.82E+03 | 1.43E−02 | 1.06E−04 |
| Lintuzumab | | 111 | 8.90E−08 | 7.06E−09 | 1.87E+05 | 1.41E+04 | 1.66E−02 | 4.10E−04 |
| | | 37 | 8.54E−08 | 1.48E−08 | 2.26E+05 | 3.83E+04 | 1.93E−02 | 6.32E−04 |
| | | 12 | 1.50E−06 | 3.16E−05 | 9.98E+03 | 2.10E+05 | 1.50E−02 | 1.41E−03 |
| | Peptide | 1000 | 2.96E−07 | 8.15E−09 | 1.23E+05 | 3.18E+03 | 3.63E−02 | 3.41E−04 |
| | 19 | 333 | 1.17E−06 | 1.65E−06 | 6.22E+04 | 8.65E+04 | 7.28E−02 | 1.58E−02 |
| | | 111 | 3.00E−06 | 1.50E−05 | 1.52E+04 | 7.60E+04 | 4.55E−02 | 5.49E−03 |
| | | 37 | 5.68E−06 | 1.53E−03 | 1.27E+04 | 3.42E+06 | 7.22E−02 | 8.62E−02 |
| | | 12 | 5.55E−08 | | 2.75E+08 | | 1.53E+01 | 4.35E+06 |
| | Peptide | 1000 | 1.98E−05 | 3.63E−05 | 2.02E+04 | 3.70E+04 | 3.99E−01 | 2.51E−02 |
| | 20 | 333 | 6.55E−06 | 1.26E−05 | 4.65E+04 | 8.95E+04 | 3.05E−01 | 2.01E−02 |
| | | 111 | 3.34E−06 | 2.16E−05 | 4.99E+04 | 3.22E+05 | 1.67E−01 | 2.43E−02 |
| | | 37 | 1.21E−06 | 2.13E−05 | 1.05E+05 | 1.85E+06 | 1.28E−01 | 4.63E−02 |
| | | 12 | 2.46E−07 | 4.04E−06 | 2.60E+06 | 4.27E+07 | 6.41E−01 | 3.47E−01 |
| Meditope | Peptide | 1000 | 7.87E−08 | 1.38E−08 | 2.31E+05 | 3.92E+04 | 1.82E−02 | 7.77E−04 |
| enabled | 8 | 333 | 1.32E−07 | 9.78E−09 | 3.92E+05 | 2.72E+04 | 5.16E−02 | 1.34E−03 |
| Panitumumab | | 111 | 2.32E−07 | 3.22E−08 | 2.83E+05 | 3.82E+04 | 6.57E−02 | 2.00E−03 |
| | | 37 | 2.79E−06 | 6.91E−05 | 3.19E+04 | 7.90E+05 | 8.91E−02 | 1.99E−02 |
| | | 12 | <1.0E−12 | 8.93E−10 | 9.55E+04 | 8.52E+04 | <1.0E−07 | |
| | Peptide | 1000 | 4.00E−06 | 1.16E−05 | 7.53E+04 | 2.15E+05 | 3.01E−01 | 1.24E−01 |
| | 19 | 333 | 3.83E−06 | 1.05E−04 | 3.11E+05 | 8.53E+06 | 1.19E+00 | 1.86E+00 |
| | | 111 | 2.15E−06 | 7.49E−05 | 3.68E+05 | 1.28E+07 | 7.93E−01 | 9.61E−01 |
| | | 37 | <1.0E−12 | | 1.46E−03 | | <1.0E−07 | |
| | | 12 | <1.0E−12 | 2.23E−08 | 4.14E+04 | 9.06E+05 | <1.0E−07 | |
| | Peptide | 1000 | 2.51E−05 | 1.40E−04 | 2.16E+04 | 1.20E+05 | 5.43E−01 | 8.20E−02 |
| | 20 | 333 | 6.81E−06 | 4.31E−05 | 1.64E+05 | 1.03E+06 | 1.12E+00 | 2.33E−01 |
| | | 111 | 5.28E−06 | 1.28E−04 | 1.30E+05 | 3.16E+06 | 6.87E−01 | 2.43E−01 |
| | | 37 | 3.63E−07 | 7.38E−06 | 3.55E+06 | 7.20E+07 | 1.29E+00 | 1.72E+00 |
| | | 12 | 3.23E−06 | 5.23E−03 | 2.65E+05 | 4.30E+08 | 8.56E−01 | 3.63E+00 |
| Cetuximab | Peptide | 1000 | 4.64E−09 | 4.23E−10 | 4.28E+05 | 3.89E+04 | 1.99E−03 | 1.75E−05 |
| I83E | 8 | 333 | 4.88E−09 | 1.46E−10 | 5.85E+05 | 1.73E+04 | 2.85E−03 | 1.18E−05 |
| | | 111 | 5.51E−09 | 9.96E−11 | 5.60E+05 | 9.87E+03 | 3.09E−03 | 1.27E−05 |
| | | 37 | 7.09E−09 | 6.37E−11 | 5.07E+05 | 4.32E+03 | 3.59E−03 | 1.02E−05 |
| | | 12 | 5.14E−09 | 9.13E−11 | 5.49E+05 | 9.38E+03 | 2.82E−03 | 1.41E−05 |
| | Peptide | 1000 | 6.71E−09 | 3.59E−10 | 3.63E+05 | 1.93E+04 | 2.44E−03 | 1.34E−05 |
| | 19 | 333 | 8.49E−09 | 1.50E−10 | 3.78E+05 | 6.59E+03 | 3.21E−03 | 9.75E−06 |
| | | 111 | 1.07E−08 | 2.97E−10 | 2.99E+05 | 7.96E+03 | 3.20E−03 | 2.50E−05 |
| | | 37 | 1.01E−08 | 1.32E−10 | 2.96E+05 | 3.67E+03 | 3.00E−03 | 1.25E−05 |
| | | 12 | 2.30E−08 | 1.75E−09 | 9.65E+04 | 7.30E+03 | 2.22E−03 | 1.40E−05 |
| | Peptide | 1000 | 1.76E−08 | 3.82E−09 | 4.16E+05 | 8.94E+04 | 7.33E−03 | 2.10E−04 |
| | 20 | 333 | 9.02E−09 | 9.87E−10 | 7.54E+05 | 8.15E+04 | 6.81E−03 | 1.20E−04 |
| | | 111 | 5.74E−09 | 3.90E−10 | 8.01E+05 | 5.31E+04 | 4.59E−03 | 6.72E−05 |
| | | 37 | 9.51E−09 | 2.38E−10 | 6.30E+05 | 1.49E+04 | 6.00E−03 | 4.80E−05 |
| | | 12 | 2.63E−09 | 1.01E−10 | 1.12E+06 | 4.05E+04 | 2.93E−03 | 3.65E−05 |
| Meditope | Peptide | 1000 | 8.95E−08 | 3.09E−09 | 9.18E+04 | 3.07E+03 | 8.21E−03 | 7.28E−05 |
| enabled | 8 | 333 | 8.45E−08 | 1.67E−09 | 1.15E+05 | 2.15E+03 | 9.74E−03 | 6.27E−05 |
| Trastuzumab | | 111 | 1.38E−07 | 8.86E−09 | 8.10E+04 | 5.05E+03 | 1.12E−02 | 1.79E−04 |
| | | 37 | 2.79E−06 | 2.23E−05 | 4.58E+03 | 3.66E+04 | 1.28E−02 | 7.18E−04 |
| | | 12 | 2.76E−06 | 6.65E−05 | 2.45E+03 | 5.89E+04 | 6.77E−03 | 2.72E−04 |
| | Peptide | 1000 | 3.49E−07 | 1.55E−08 | 7.83E+04 | 3.27E+03 | 2.73E−02 | 4.17E−04 |
| | 19 | 333 | 8.69E−07 | 4.37E−07 | 3.84E+04 | 1.90E+04 | 3.34E−02 | 3.10E−03 |
| | | 111 | 1.38E−05 | 6.43E−04 | 2.38E+03 | 1.11E+05 | 3.29E−02 | 7.99E−03 |
| | | 37 | 2.19E+12 | | 2.11E+03 | 4.30E+30 | 4.61E+15 | |
| | | 12 | 6.65E+14 | | 8.14E+02 | | 5.41E+17 | |
| | Peptide | 1000 | 1.97E−05 | 4.04E−05 | 2.06E+04 | 4.22E+04 | 4.07E−01 | 2.86E−02 |
| | 20 | 333 | 6.60E−06 | 1.33E−05 | 6.16E+04 | 1.24E+05 | 4.06E−01 | 2.80E−02 |
| | | 111 | 2.36E−06 | 1.82E−05 | 2.18E+05 | 1.68E+06 | 5.14E−01 | 1.27E−01 |
| | | 37 | 5.94E−06 | 1.61E−03 | 8.33E+04 | 2.26E+07 | 4.95E−01 | 5.86E−01 |
| | | 12 | 8.79E−07 | 1.97E−04 | 8.22E+02 | 1.84E+05 | 7.23E−04 | 2.38E−04 |

1. A peptide comprising an amino acid sequence of CQFDLSTRRLRC (SEQ ID NO: 1) or SEQ ID NO: 1 with one or two amino acid additions, deletions and/or substitutions, wherein the amino acid sequence comprises one or more modifications of at least one amino acid residue selected from the group consisting of Phe3, Leu5, Arg8, Arg9, and/or Leu10, and further wherein the modification comprises incorporation of a photoreactive functional group capable of forming a covalent bond upon irradiation with UV light.

2. A peptide comprising the amino acid sequence of CQFDLSTRRX¹RC (SEQ ID NO: 7) or CQYNLSSRAX¹KC (SEQ ID NO: 13), or SEQ ID NO: 7 or 13 with one or two amino acid additions, deletions and/or substitutions, wherein X¹ comprises a photoreactive functional group capable of forming a covalent bond upon irradiation with UV light.

3. The peptide of claim 1, wherein the sequence further comprises -GGGK at the C-terminus.

4. The peptide of claim 1, wherein the peptide is cyclic.

5. The peptide of claim 2, wherein X¹ is selected from the group consisting of L-2-amino-4,4-azi-pentanoic acid, L-2-amino-5,5-azi-hexanoic acid, azido-phenylalanine, and para-benzoylphenylalanine.

6. The peptide of claim 1, further comprising one or more modifications selected from one or more of head-to-tail lactam cyclic peptides, a linear peptide, an incorporation of an unnatural amino acid, a shortening or lengthening of a bond, and an incorporation of hydratable carbonyl functionality.

7. A non-covalent antibody-meditope complex, comprising the peptide of claim 1 and an antibody comprising a meditope-enabled cavity.

8.-9. (canceled)

10. An antibody-meditope conjugate, comprising a cyclic meditope comprising the amino acid sequence of CQFDLSTRRXRC (SEQ ID NO: 7) or CQYNLSSRAXKC (SEQ ID NO: 13), each sequence having a disulfide bridge between C1 and C12, or SEQ ID NO: 7 or 13 with one or two amino acid additions, deletions and/or substitutions and an antibody comprising a meditope-enabled cavity, wherein the side chain of amino acid X of the meditope is covalently bonded to the antibody, and wherein a disulfide bridge between C1 and C12 is closer to the entrance of the meditope-enabled cavity than the majority of amino acids 2-11 of the meditope.

11. (canceled)

12. The antibody-meditope conjugate of claim 10, wherein the side chain of amino acid X is covalently bonded to an amino acid side chain of the antibody.

13. The antibody-meditope conjugate of claim 12, wherein the side chain of amino acid X is covalently bonded to the antibody backbone.

14. The antibody-meditope conjugate of any one of claim 10, wherein the side chain of X is covalently bonded to the antibody via a linker -L- or -L-L-, wherein each L is independently substituted or unsubstituted alkylene, substituted or unsubstituted cycloalkylene, substituted or unsubstituted arylene, substituted or unsubstituted heterocyclylene or substituted or unsubstituted heteroarylene.

15. The antibody-meditope conjugate of any one of claim 10, wherein the meditope further comprises at least one active agent covalently bound thereto, optionally via a linker.

16. The antibody-meditope conjugate of any one of claim 10, wherein the meditope further comprises an active agent covalently bound to both the N-terminus and the C-terminus, optionally via a linker.

17. The antibody-meditope conjugate of claim 15, wherein the active agent is a therapeutic agent, a diagnostic agent, or a detectable agent.

18. The antibody-meditope conjugate of any one of claim 8, wherein the antibody is cetuximab, gemtuzumab, lintuzumab, trastuzumab, panituzumab, clivatuzumab, ocaratuzumab, or a functional fragment thereof comprising the meditope-enabled cavity.

19. The antibody-meditope conjugate of any one of claim 8, wherein the antibody comprises at least one of amino acids as described in Table 1 and/or Table 2.

20. The antibody-meditope conjugate of any one of claim 8, wherein the antibody comprises at least one of amino acids LC 101 (G), LC 105 (E), HC 44 (G), and/or HC 155 (E).

21.-23. (canceled)

24. A method of administering an active agent to a patient in need thereof, comprising administering the antibody-meditope conjugate of any one of claim 10, wherein the antibody-meditope conjugate comprises an active agent bound thereto.
